(19) United States
(12) Patent Application Publication
(10) Pub. No.: US 2013/0122050 A1
(43) Pub. Date: May 16, 2013

(54) COMPOSITIONS AND METHODS FOR TREATING DIABETES USING LISOFYLLINE ANALOGS AND ISLET NEOGENESIS ASSOCIATED PEPTIDE

(71) Applicant: DiaKine Therapeutics, Inc., Charlottesville, VA (US)

(72) Inventors: Mary Ann Latona NADLER, Charlottesville, VA (US); Jerry L. NADLER, Charlottesville, VA (US)

(73) Assignee: DIAKINE THERAPEUTICS, INC., Charlottesville, VA (US)

(21) Appl. No.: 13/644,817

(22) Filed: Oct. 4, 2012

Related U.S. Application Data
(63) Continuation of application No. 12/865,062, filed on Jul. 28, 2010, now abandoned.
(60) Provisional application No. 61/025,232, filed on Jan. 31, 2008.

(51) Int. Cl. A61K 38/10; A61K 31/522; A61K 31/695; A61K 31/5377

(52) U.S. Cl. CPC A61K 38/10; A61K 31/5377; A61K 31/522; A61K 31/695
USPC 424/278.1; 514/6.9

(57) ABSTRACT

Pharmaceutical compositions and methods are provided for treating diabetes and/or restoring β-cell mass and function in a mammal in need thereof. Type 1 diabetes mellitus (T1 DM) is an autoimmune disorder characterized by immune damage to pancreatic beta-cells. Lisofylline analogs (LSF analogs) are immunomodulators that reduce interlukin 12 signaling and reduce the onset of T1 DM in non-obese diabetic (NOD) mice. A combination therapy with both LSF analog (pretreatment) and INGAP provides protection from autoimmune destruction. The concomitant or combination of an LSF analog and INGAP after pre-treatment with an LSF analog is an effective therapy for a disease or condition resulting from the loss of pancreatic islet cells or insulin production in a mammal.

### FIELD OF THE INVENTION

The invention relates to the use of pharmaceutical compositions and methods for using same for (1) restoring β-cell mass and function in an individual in need thereof; (2) preventing the development of, or reversing, Type 1 diabetes mellitus (T1DM) in an individual in need thereof; (3) for preventing the development of, or reversing, latent autoimmune diabetes of adults (LADA) in an individual in need thereof; and/or (4) for treating Type 2 diabetes mellitus (T2DM) by increasing the number of functional insulin-producing cells (e.g., β-cells) in an individual in need thereof.

### BACKGROUND OF THE INVENTION

Insulin is a hormone produced in the pancreas by beta cells (β-cells). The function of insulin is to regulate the amount of glucose (sugar) in the blood, which enters cells through receptors that accept insulin and allow glucose to enter. Once inside, glucose can be used by an organism as fuel. Excess glucose is stored in the liver and muscles in a form called glycogen. When blood glucose levels are low, the liver releases glycogen to form glucose. Without insulin, glucose has difficulty entering cells, which in turn, causes myriad deletrious effects.

Since about 1922, insulin has been the only available therapy for the treatment of type diabetes and other conditions related to lack of or diminished production of insulin. Despite decades of research and the advent of pancreatic islet cell transplantation in 1974 and newer claims of success resulting from the Edmonton Protocol for islet cell transplantation, the success has not been replicated in the United States. At four years post-transplant, fewer than 10% of patients who have received islet cell transplants remain insulin independent. Additionally, despite new immune suppression protocols, there is an 18% rate per patient of serious side effects.

Diabetes (Type 1, 2 or LADA) is one of the most common metabolic diseases affecting hundreds of millions of individuals worldwide. In persons with diabetes, the pancreas produces no insulin, too little insulin to control blood sugar, or defective insulin. Without insulin, these symptoms progress to dehydration, resulting in low blood volume, increased pulse rate, and dry, flushed, skin. In addition, ketones accumulate in the blood faster than the body is able to eliminate them through the urine or exhaled breath. Respiration becomes rapid, and shallow and breath has a fruity odor. Other symptoms indicating a progression towards diabetic ketoacidotic coma (DKA) include vomiting, stomach pains, and a decreased level of consciousness. The disease leads to serious complications, including hyperglycemia, macroangiopathy, microangiopathy, neuropathy, nephropathy and retinopathy. As a result, diabetes adversely affects the quality of life.

There are two forms of diabetes mellitus: (1) insulin dependent or T1DM (a.k.a., Juvenile Diabetes, Brittle Diabetes, Insulin Dependent Diabetes Mellitus (IDDM)) and (2) non-insulin-dependent or Type II diabetes (a.k.a., NIDDM). T1DM develops most often in young people but can appear in adults. T2DM develops most often in middle aged and older adults, but can appear in young people. Diabetes is a disease believed to be derived from multiple causative factors and characterized by elevated levels of plasma glucose (hyperglycemia) in the fasting state or after administration of glucose during an oral glucose tolerance test.

T1DM is an autoimmune disease condition characterized by high blood glucose levels caused by a total lack of insulin, i.e., a complete loss of pancreatic β-cell function and mass. T1DM occurs when a person's immune system attacks the insulin producing β-cells in the pancreas and destroys them. It is believed that the interleukin 12 (IL-12) family of cytokines and downstream activation of Signal Transducers and Activators of Transcription (STAT) family members, e.g., STAT-4, which are believed to be regulators of T cell differentiation involved in immune responses, play a major role in the processes that lead to autoimmune β-cell destruction. The pancreas then produces little or no insulin. The most common T1DM symptoms experienced include excessive thirst (polydipsia), frequent urination (polyuria), extreme hunger (polyphagia), extreme fatigue, and weight loss. These symptoms are caused by hyperglycemia and a breakdown of body fats. Persons diagnosed with T1DM typically exhibit blood sugar levels over 300 mg and ketones present in their urine. Restoration of β-cell mass and insulin production can fully reverse the diabetic state. Evidence suggests that people with long standing T1DM have β-cells that continue to form but are undesirably destroyed by continued autoimmune destruction. Therefore, pharmaceutical compositions and methods for arresting autoimmune β-cell damage would provide an effective way to restore normal β-cell mass levels and reverse or cure T1DM.

LADA is a newly recognized subset of T1DM and is thought to account for up to 10%-20% of all cases of diabetes. LADA is often present in people initially diagnosed with T2DM. Although it has characteristics similar to adult onset T1DM, the beta-cell destruction is considered to be less aggressive in its progression.

T2DM results from a combination of insulin resistance and impaired insulin secretion but ultimately many people with T2DM show markedly reduced pancreatic β-cell mass and function which, in turn, causes Type 2 diabetic persons to have a “relative” deficiency of insulin because pancreatic β-cells are producing some insulin, but the insulin is either too little or isn't working properly to adequately allow glucose into cells to produce energy. Recent autopsy studies have shown clear evidence of ongoing β-cell death (apoptosis) in people with T2DM. Therefore, therapeutic approaches to arrest β-cell death could provide a significant treatment for reversing or curing T2DM.

Uncontrolled T2DM leads to excess glucose in the blood, resulting in hyperglycemia, or high blood sugar. A person with T2DM experiences fatigue, increased thirst, frequent urination, dry, itchy skin, blurred vision, slow healing cuts or sores, more infections than usual, numbness and tingling in feet. Without treatment, a person with 12DM will become dehydrated and develop a dangerously low blood volume, if T2DM remains uncontrolled for a long period of time, more serious symptoms may result, including severe hyperglycemia (blood sugar over 600 mg) lethargy, confusion, shock, and ultimately “hyperosmolar hyperglycemic non-ketotic coma.” Persistent or uncontrolled hyperglycemia is associated with increased and premature morbidity and mortality. As such, therapeutic control of glucose homeostasis, lipid metabolism, obesity, and hypertension are critically important in the clinical management and treatment of diabetes mellitus.

The object of diabetes treatments is to prevent the occurrence of the above-mentioned chronic complications, slow disease progression by improving hyperglycemic status, or reversing/curing it. Conventional methods for treating diabetes have included administration of fluids and insulin in the case of Type I diabetes and administration of various hypoglycemic agents in the case of Type II diabetes. Hypoglycemic agents such as insulin preparations, insulin secretagogues, insulin sensitizers and α-glucosidase inhibitors have been widely applied as the method for the clinical treatment. Examples include acarbose (PrecoseJ), glimeprimide (AmarylJ), metformin (Glucophage7), nateglinide (Starlix7), pioglitazone (Actos7), repaglinide (PrandinJ), rosiglitazone (Avandia7), sulfonylureas, Orlistat (Xenical7), exenatide (Byetta), and the like. Many of the known hypoglycemic agents, however, exhibit undesirable side effects and are toxic in certain cases. For example, in the case of the diabetic patients with seriously lowered pancreatic insulin secretion, effectiveness of insulin secretagogues and insulin sensitizers is diminished. Similarly, in the case of the diabetic patients whose insulin resistance is significantly high, effectiveness of insulin preparations and insulin secretagogues is diminished.

In principle, diabetes mellitus could be “cured” by a successful transplant of the tissue containing cells that secrete or produce insulin, i.e., the islets of Langerhans. Transplantation of insulin producing cells (a.k.a., islets) has been tried as a method to reverse or cure T1DM, but there are significant risks associated with the surgery and with the toxic immunosuppression type drugs that need to be taken to prevent or mitigate allograft rejection and autoimmune reoccurrence. Immunosuppression drugs act by reducing the activity of a recipient's immune system so that the transplanted insulin producing cells are not rejected. Such immunosuppression, however, entails substantial risks and there are considerable difficulties attendant in minimizing the antigenic differences (matching) between a donor and a recipient that increases the costs and reduces the availability of this mode of therapy. In addition, conventional immunosuppression is generally not successful in enabling islet transplantation. Moreover, there are over 1 million people with T1DM in the United States today, but the supply of cadaveric pancreatic tissue for islets is limited. For instance, only 6,000 organs are available per year and 2 or 3 organs are needed to provide enough islets to reverse T1DM in one person. Therefore, providing a new source of functioning (insulin producing) β-cells is urgently needed. In addition, if a diabetic patient's own cells (pancreatic or other cell types) could be genetically engineered or induced to grow and differentiate into functioning β-cells, then there would be little or no need to use toxic anti-rejection medications. As previously mentioned, there continues to be the capacity for new β-cell formation in people with T1DM. However, continued autoimmunity leads to active destruction of any newly formed or transplanted β-cells. Development of new immunomodulating agents would provide a new way to fully reverse β-cell disfunction in T1DM without the need for islet cell transplantation or toxic anti-rejection immunosuppressants. Further, the pre-treatment followed by combination therapy approach provided by the invention would be a major improvement in cellular replacement therapy by reducing the amount of transplanted cells needed to reverse or cure T1DM, facilitating the increase viability and growth of insulin producing cells, thereby improving success rates.

Unfortunately, insulin therapy does not treat the underlying mechanisms disease resulting in T1DM and other such conditions in which there is diminished endogenous insulin production. The therapies, methods, modalities, and treatments described herein are the first to address the many facets of the cause and complications of diabetes. The unique therapies provided by the invention encompass diverse aspects diabetology, metabolism, and immunology. These therapies include those that bring the many different hormones, in addition to insulin, that are diminished or absent in T1DM. The methods of the invention provide for the regeneration of new insulin producing cells and immuno-modulation that together serve to ameliorate, diminish, or abolish the need for insulin among patients with T1DM and other conditions associated with inadequate insulin production and secretion,

### Glucagon-Like Peptide (GLP-1) and Gastric Inhibitory Polypeptide (GLP)

Incretins are intestinal hormones released after meal ingestion that stimulate insulin secretion. GLP-1 is a 300-amino-acid (peptide) incretin synthesized in the small and large intestine by the L-type cells of the gastroenteropancreatic endocrine system and is released in response to food ingestion. GLP-1 enhances glucose-stimulated postprandial insulin secretion, stimulates insulin gene expression and proinsulin biosynthesis, inhibits pancreatic glucagons release, gastric emptying, and acid secretion. GIP is another insulin releasing hormone secreted from endocrine cells in the intestinal tract in response to food intake. Together with autonomic nerves, GLP-1 and GIP play a vital supporting role to the pancreatic islets in the control of blood glucose homeostasis and nutrient metabolism.

GLP-1 shows potent insulinotropic action in both diabetic and nondiabetics. GLP-1 causes expansion of beta-cell mass via proliferation of insulin-producing cells. GLP-1 shows an ability to stimulate β-cell neogenesis in streptozotocin (STZ)-treated newborn rats, resulting in persistent improvement of glucose homeostasis to adult age. GLP-1 induces differentiation of islet duodenal homeobox-1-positive pancreatic ductal cells into insulin-secreting cells by enhancing expression of transcription factors POX-1 and HNF3. GLP-1 has been shown to promote functional maturation of fetal porcine β-cells and islet cell growth in a Type 2 diabetic rat model. Cloning and functional expression of GLP-1 receptors are completed in human islets. GLP-1 receptor signaling directly modifies the susceptibility of β-cells to apoptotic injury that may be the potential mechanism linking to preservation and enhancement of β-cell mass and function. GLP-1 receptor signaling, however, does not seem essential for glucose-stimulated insulin secretion, as shown in GPL-1 receptor knockout mice, which suggests that the functional signaling of GLP-1 in β-cells may be in addition to the one initiated by glucose.

GLP-1 has been studied as a potential drug for the management of diabetes for two reasons: (i) its effect on β-cell growth; and (ii) its insulin-stimulating effect with minimal risk of hypoglycemia and absence of effect on insulin action in non-diabetic humans. In limited clinical trails, GPL-1 is effective in treating Type 2 diabetic patients, showing a significant improvement in postprandial glycemic control and normalization of fasting hyperglycemia due to its ability of insulinotropic activity.

GIP is released from intestinal endocrine K-cells into the bloodstream following ingestion of carbohydrate, protein and particularly fat. GIP's major physiological role is generally believed to be that of an incretin hormone that targets pancreatic islets to enhance insulin secretion and help reduce postprandial hyperglycemia, GIP acts through binding to specific G-protein coupled GIP receptors located on pancreatic beta-cells (Wheeler, M. B. et al., 1995. Endocrinology 136:4629-4639). GIP has been shown to stimulate β-cell proliferation synergistically with glucose in the islet INS-1 cell line, in association with induction of MAPK and PI 3-kinase. Similarly, GIP exerts anti-apoptotic actions in studies using INS-1 β-cells. Like glucagon-like peptide-1 (GLP-1), the ability to stimulate insulin secretion plus other potentially beneficial actions on pancreatic beta-cell growth and differentiation have led to much interest in using GLP-1 or GIP and analogs thereof for the treatment of T2DM.

Neither, GLP-1 nor GIP, however, appear suitable for therapeutic use in chronic disorders, such as T2DM because GLP-1 and GIP are rapidly cleared from blood circulation (half life of about 1.5 min.) by the ubiquitous enzyme dipeptidyl peptidase-IV (DPP-IV). Exogenously administered GLP-1 is also rapidly degraded. This metabolic instability limits the therapeutic potential of native GLP-1 and GIP.

### Exendin-4 (Ex-4)

As an analog of GLP-1, Ex-4 was first isolated from the salivary secretions of a South American lizard known as the Gila monster (Heloderma suspectum). Ex-4 consists 39-amino acids with 53% structural homology to mammalian GLP-1. Ex-4 is capable of binding to both human and rat GLP-1 receptors and shows similar pharmacological and biological properties of GLP-1. As a more potent agent than GLP-1, Ex-4 is strongly capable of increasing β-cell mass by enhancing both cell replication and neogenesis, and by inhibiting the apoptosis of β-cells.

In spite of similarities, Ex-4 differs from GLP-1: (i) Ex-4 is resistant to DPP-IV cleavage, resulting in a long-lasting biological function that is potentially suitable for therapeutic use; (ii) Ex-4 has greater insulinotropic efficacy; and (iii) although both GLP-1 and Ex-4 have similar effects to augment insulin-stimulated glucose uptake and metabolism in skeletal muscle. Ex-4 also increases glucose uptake in adipocytes. Ex-4 may also use different signaling pathways, possibly through a receptor other than the GLP-1 receptor. This may render Ex-4 more effective in reducing blood glucose by simultaneously stimulating β-cell insulin secretion and increasing glucose utilization in both skeletal muscle and fat tissue. Ex-4 has also been studied for treatment of T2DM, as an additive to existing treatments (such as metformin and/or sulfonylurea) to control hyperglycemia in Type 2 diabetic patients. An injectable synthetic form of Ex-4 (Byetta® (exenatide) sold by Amylin Pharmaceuticals, Inc.) has been recently approved for use in treating T2DM as an adjunctive therapy to improve blood sugar control.

A study recently showed that Ex-4, along with anti-lymphocyte serum (ALS), reversed hyperglycemia in previously overt diabetic NOD (Non-Obese Diabetic) mice. In this study, GLP-1 alone showed no effect to hyperglycemia in NOD mice, indicating that controlling auto-activated lymphocytes by ALS was required to achieve remission of euglycemia. However, ALS is a potent immunosuppressant that causes general dysfunction in all types of lymphocytes. Long-term use of ALS has been known to lead to the risk of tumorigenesis and other severe infectious diseases due to general immune deficiency. Therefore, ALS and other immunosuppressant drugs have not been shown to be clinically useful in treating diabetes.

### Islet Neogenesis Associated Protein (INGAP)

One islet stimulating hormone is islet Neogenesis Associated Peptide (“INGAP” or “INGAP peptide”). INGAP (including analogs and derivatives thereof) is a member of the Reg3 family of pancreatic proteins and can induce new islet formation and restore euglycemia in streptozotocin-induced diabetic mice. INGAP is a 15 amino acid peptide with the following sequence: Ile-Gly-Leu-His-Asp-Pro-Ser-His-Gly-Thr-Leu-Pro-Asn-Gly-Ser (MW 1501.4). INGAP peptide is the active core of a 168 kDa protein which has demonstrated islet neogenesis activity in several species. INGAP peptide causes the growth of new, fully-functioning islets from progenitor cells. To date, the peptide has been shown to increase β-cell mass and insulin production both in various animal models and in cultures of human tissue. It has also been shown to be safe in clinical studies and an open investigational New Drug Application (IND) is on file with the United States Food and Drug Administration (FDA). However, because T1DM is associated with loss of β-cell mass resulting from an autoimmune response, administration of INGAP peptide alone does not appear to be a optimally suitable long-term solution because autoimmune mechanisms that destroy original β-cells would also hinder or preclude the buildup of sufficient β-cell mass under a regimen involving INGAP peptide as a monotherapy. Suitable preparations of INGAP may be obtained in accordance with, inter alia, the teachings in U.S. Reissue Pat. Nos. 39299, 39351 and 39062, the entire disclosures of which are incorporated herein by reference.

Lisofylline analogs (“LSF analogs”) are believed to be capable of preventing autoimmune diabetes, partially due to its anti-inflammatory function by reducing inflammatory cytokine production, including tissue necrosis factor-α (TNF-α) interferon-γ (IFN-γ), and interleukin-1β (IL-1β). LSF analogs effectively suppresse T cell activation and differentiation via inhibition of the signal transducer and activator of transcription-4 (STAT4)-mediated interleukin-12 (ILL-2) signaling, which can prevent autoimmune diabetes and protect transplanted islets from autoimmune destruction. LSF analogs also have anti-inflammatory actions, contributing to preservation of islet viability and function. LSF analogs maintain beta-cell insulin secretory function in the presence of inflammatory cytokine insult and regulates immune cellular function to suppress autoimmunity. LSF analogs also enhance insulin secretion in insolated islets and in transformed beta-cell lines in response to glucose stimulation in vitro and prevents the onset of autoimmune diabetes. LSF analogs alone do not reverse autoimmune diabetes in non-obese diabetic (NOD) mice when hyperglycemia became detectable, however, LSF analogs alone are able to stabilize but not normalize blood glucose levels in some cases as long as the treatment was continued. This suggests that LSF analogs alone does not appear to be capable of inducing remission of diabetes. LSF analogs have also been shown to be safe in clinical studies and an open IND is also on file with the FDA.

To date, there has been no single/combination or concomitant therapy or treatment protocol that has been successfully used to treat the underlying disease mechanisms of T1DM or conditions in which there is a lack of or diminished insulin production. There remains a need for new methods and pharmaceutical compositions for treating T1DM mellitus. Especially needed are methods and compositions that can also treat the many other conditions in which the lack of or diminished insulin production has a causative role or contributes to the symptoms of patients in need of treatment. At present, there appears to be no treatment that ameliorates the symptoms of T1DM by targeting the underlying disease mechanism. There also remains a need for more effective pharmaceutical compositions and methods that utilize immunomodulating agents as a pretreatment followed by the same or similar immunomodulating agents in combination with a β-cell growth and/or differentiating factor to restore normal β-cell mass and/or function in subjects suffering from diabetes.

### SUMMARY OF THE INVENTION

Now it has been surprisingly found that pre-treatment with a biological/immune response modifier (immunomodulating) or anti-inflammatory agent (e.g., small molecule, antibody, peptide or gene therapy reagent) that effectively blocks autoimmune response or cytokine formation in a mammal (e.g., structurally related analogs of LSF, as further described below), followed by treatment with a combination of the biological response modifier and any compound or agent (e.g., small molecule or peptide) (e.g., INGAP, Ex-4, Byetta®, etc.) that facilitates better growth and/or differentiation of pancreatic β-cells or any insulin producing cell is useful for restoring normal β-cell mass and/or function; preventing the development of, or reversing, T1DM, Latent Autoimmune Diabetes of Adults (LADA), and/or T2DM; and increasing the number of functional insulin producing cells in an individual in need thereof as compared with previous pharmaceutical compositions and methods. In one aspect, the invention provides for the use of compounds or agents that can block cytokine signaling or formation and thereby prevent autoimmune damage to regenerated/emerging new insulin producing cells. Without using an agent to block the autoimmune process, β-cell differentiation and/or growth promoting agents will not be clinically effective because simultaneous regeneration of β-cells and prevention of autoimmune reactions would not be realized. The success of pre-treatment step described herein is wholly unexpected.

In an exemplary embodiment, the invention provides pharmaceutical compositions that may be used in a method for the prevention and treatment (including reversal and cure) of mammals (including humans and animals) suffering from diseases or conditions caused by, or associated with, diabetes mellitus (Type 1, LADA and Type 2), hyperglycemia, dyslipidemia, hyperlipidemia, hypercholesterolemia, hypertriglyceridemia, hyperinsulinemia, diabetic complications, glucose intolerance, obesity or the like.

An exemplary method of the invention comprises:

(1) administering to a mammal, e.g., a human patient or animal, a pharmaceutically or therapeutically effective amount of a biological/immune response modifier (immunomodulating) or anti-inflammatory agent (e.g., small molecule, antibody, peptide or gene therapy reagent) that effectively blocks autoimmune response in a mammal by inhibiting the activity or expression of inflammatory cytokines such as, for example, IL-12, IL-23 or IL-27, or STAT-4 (such as, for example an LSF analog); followed by

(2) the administration of preventative- or therapeutically-effective amount of a pharmaceutical composition comprising, in admixture with a pharmaceutically acceptable carrier, diluent, excipient, adjuvant or vehicle: (a) the same or different a biological/immune response modifier or anti-inflammatory agent and (b) any compound or agent (small molecule or peptide) that facilitates growth and/or differentiation of pancreatic β-cells or any insulin producing cell, either alone or in admixture with a diluent or in the form of a medicament (such as, for example, Glucagon Like Peptide-1 (GLP-1); GP-1 receptor analogs, Exendin-4; Exenatide/BYETTA™, Gastric Inhibitory Peptide/Glucose-Dependent Insulinoptropic polypeptide (GIP): compounds homologous to GLP-1 such as Liraglutide (NN2211); Dipeptidyl Peptidase-4 inhibitors, which inhibit the breakdown of GLP-1; Gastrin, Epidermal Growth Factor; and Epidermal Growth Factor Analogs; Human prolslet Peptide (HIP) or an analog or a derivative thereof; and INGAP (hamster derived, humanized and/or analogs thereof)).

Other suitable biological response modifiers may include, without limitation: Anti CD-3 antibodies (hOKT3γ1 (Ala-Ala and ChAglyCD3); Sirolimus (Rapamycin); Tacrolimus (FK506), a heat-shock protein 60 (DIAPEP277™); anti-Glutamic Acid Decarboxylase 65 (GAD65) vaccines; Mycophenolate Mofetil alone or in combination with Daclizumab (an anti-CD20 agent); Rituximab, Campath-1H (Anti-CD52 Antibody) and Viaamin D, IBC-VSO vaccine; metabolically inactive forms of insulin designed to prevent pancreatic beta-cell destruction; interferon-α vaccination using CD4⁺CD26⁺ antigen-specific regulatory T cells or a similar agent is used in the combination therapy approaches to utilizing regulatory T cells either directly or through the use of immunotherapy to arrest the destruction of insulin-producing cells.

It is believed that pre-treatment to inhibit inflammatory cytokine (e.g., IL-12) overproduction, or inhibiting the production of cytokines such as IL-23 and IL-27 which promote STAT-4 activation and autoimmune disorders such as T1DM and LADA development is critical. Overproduction of inflammatory cytokines such as IL-6 IL-1, beta interferon gamma, TNF-α, etc. and the resultant excessive Th1 type responses can be suppressed by modulating IL-12, IL-23 and/or IL-27 production. Therefore, compounds (e.g., small molecule, antibody, peptide or gene therapy reagent) that down-regulate IL-12, IL-23 and/or IL-27 production can be used as a pro-treatment to “quiet” immune system without the deleterious side effects experienced with immunosuppressants. After pre-treatment, various combination treatments, including those disclosed in U.S. Pat. No. 7,393,919 or U.S. Patent Application No. 2006-0198839 may be employed.

Exemplary biological/immune response modifying (immunomodulating) or anti-inflammatory compounds or agents include, without limitation, members of the group consisting of: LSF analogs described in W0/00/61583 (corresponding to U.S. Pat. No. 6,774,130 (the entire disclosure of which is incorporated herein by reference) or any other small molecule or peptide or method capable of blocking interleukin 12, interleukin 23 or activation and/or expression of STAT-4, as may be further described below.

Lisofylline (a.k.a. 1-(5-R-hydroxyhexyl)-3,7-dimethylxanthine) is a synthetic, modified xanthine based compound have the following structural formula:

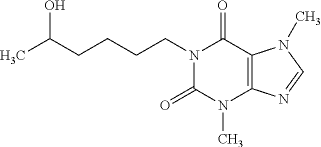 [SMILES: CC(O)CCCCn1c(=O)c2c(ncn2C)n(C)c1=O]

Without wishing to be bound by any theory of operation or mode of action, the analogs of lisofylline described below exhibit anti-inflammatory function by reducing inflammatory cytokine production or downstream effects (including, without limitation, IL-12, IL-23, IL-27, TNF-α, IFN-γ, IL-6 and IL-1β), selectively suppressing neutrophil and leukocyte adhesion and phagocytic activity, and decreasing neutrophil migration and degranulation during sepsis. More significantly, LSF analogs allow retention of beta-cell insulin secretory function after inflammatory cytokine insult and regulates immune cellular function to prevent autoimmunity. In addition, LSF analogs also exhibit the ability to ameliorate hemorrhage-induced tissue injury and to preserve tissue function during decreased blood flow or in poorly ventilated conditions. LSF analogs also inhibit phosphatidic acid formation to prevent oxidant-mediated capillary teak, thus reducing capillary barrier damage caused by oxidative stress. All of these characteristics render the LSF analogs described below capable of improving the clinical outcome by their use as a pre-treatment prior to administration of mono or combination therapies.

The pharmaceutical compositions useful in the invention may conveniently be provided, or is otherwise envisioned in the form of formulations suitable for parenteral (including intravenous, intramuscular and subcutaneous) nasal, oral administration or pulmonary via a inhalation device. In some cases, it will be convenient to provide a biological/immune response modifier or anti-inflammatory agent, as described herein, and any compound or agent (small molecule or peptide) that facilitates growth and/or differentiation of pancreatic β-cells or any insulin producing cell, each in a single composition or solution for administration together. A suitable administration format may best be determined by a medical practitioner for each patient individually. Suitable pharmaceutically acceptable carriers and their formulation are described in standard formulation treatises, e.g., Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2S (1998).

Exemplary compounds or agents that may be used in accordance with the principles of the invention for inducing pancreatic β-cell or insulin producing cell growth and/or differentiation include, but are not limited to, members of the group consisting of: glucagon-like peptide-1 (GLP-1) and long-acting, DPP-IV-resistant GLP-1 analogs thereof, GLP-1 receptor agonists, gastric inhibitory polypeptide (GIP) and analogs thereof (e.g., which are disclosed in U.S. Patent Publication No. 20050233969), dipeptidyl peptidase IV (DPP-IV) inhibitors, insulin preparations, insulin derivatives, insulin-like agonists, insulin secretagogues, insulin sensitizers, biguanides, gluconeogenesis inhibitors, sugar absorption inhibitors, renal glucose re-uptake inhibitors, β3 adrenergic receptor agonists, aldose reductase inhibitors, advanced glycation end products production inhibitors, glycogen synthase kinase-3 inhibitors, glycogen phosphorylase inhibitors, antilipemic, agents, anorexic agents, lipase inhibitors, antihypertensive agents, peripheral circulation improving agents, antioxidants, diabetic neuropathy therapeutic agents, and the like.

Accordingly, the invention provides use of the pharmaceutical compositions and agents described herein in conjunction with (1) methods for restoring β-cell mass and function in an individual in need thereof; (2) methods for preventing the development of, or reversing, T1DM in an individual in need thereof; (3) methods for preventing the development of, or reversing, latent autoimmune diabetes of adults (LADA) in an individual in need thereof; and (4) methods for treating T2DM by increasing the number of functional insulin producing cells (e.g., β-cells) in an individual in need thereof.

The above compounds and agents used in the pharmaceutical composition of the invention may be purchased from conventional sources, may be readily isolated from and purified (isolated) from natural sources or may be synthesized using conventional techniques known to the skilled artisan using readily available starting materials.

Other technical features and advantages of the invention will be set forth, in part, in the description that follows, or may be learned from practicing or using the invention. The advantages of the invention may be realized and attained by means of technical features described below and pointed out in the appended claims. It is to be understood that the foregoing general description and the following detailed description are merely exemplary and explanatory and should not to be viewed as being restrictive of the invention, as claimed.

### BRIEF DESCRIPTION OF THE DRAWINGS

The accompanying drawings, which are incorporated in, and constitute a part of the specification, illustrate or exemplify embodiments of the invention and, together with the description, serve to explain the principles and features of the invention.

FIG. 1 depicts a prophetic therapy regimen of the invention involving INGAP peptide and an LSF analog for building and preserving β-cell mass and increasing insulin production in a subject (e.g., a mouse).

FIG. 2 depicts a prophetic timelines for exemplifying treatments using LSF analogs and INGAP.

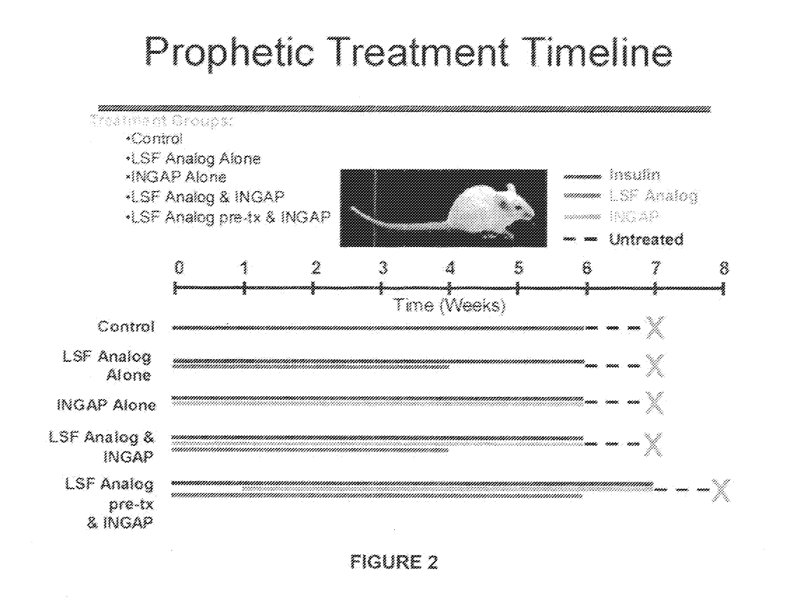

### DESCRIPTION OF EXEMPLARY EMBODIMENTS

All patents, patent applications and literatures cited or referenced in this description are incorporated herein by reference in their entirety in the case of inconsistencies, the present disclosure, including definitions, will control.

The pharmaceutical compositions and methods of the invention comprise the pre-treatment with a biological/immune response modifier or anti-inflammatory agent (e.g., small molecule, antibody, peptide or gene therapy reagent) that effectively blocks comprising a biological response modifier and a β-cell growth factor in admixture with a pharmaceutically acceptable carrier, adjuvant or vehicle, wherein the pharmaceutical composition blocks or prevents the autoimmune response in a mammal by inhibiting the activity or expression of cytokines such as interleukins 12, 23 or 27, or members of the family of Signal Transducers and Activators of Transcription (STAT), preferably STAT-4, which are believed to be regulators of T cell differentiation involved in immune responses, followed by (2) administration of a combination of (a) the same or different biological/immune response modifier or anti-inflammatory agent, as described above and (b) any compound or agent (small molecule or peptide) that induces growth and/or differentiation of pancreatic β-cells or any insulin producing cell.

Exemplary agents that could be used to induce pancreatic β-cell or insulin producing cell growth and/or differentiation following LSF-analog pretreatment include, but are not limited to, one or more members of the group consisting of:
glucagon-like peptide 1 (GLP-1);
long-acting, DPP-IV-resistant GLP-1 analogs thereof, including, without limitation, members of the group consisting of Exendin-4 (Ex-4), Exenatide (Byetta®, Amylin Pharmaceuticals), Exenatide LAR and related analogs disclosed in U.S. Pat. No. 5,424,286, U.S. Pat. No. 6,858,576, U.S. Pat. No. 6,872,700, U.S. Pat. No. 6,902,744, U.S. Pat. No. 6,956,026, U.S. Pat. No. 6,899,883 and U.S. Pat. No. 6,989,148 (the entire disclosures of which are incorporated herein by reference), Liraglutide (a.k.a., NN2211 or Arg(34)Lys(26)-(N-epsilon-(gamma-Glu(N-alpha-hexadecanoyl))-GLP-1(7-37)) (Novo Nordisk), CJC-1131 (Conjuchem Inc.), Albugon (Human Genome Sciences), LY-548806 (Eli Lilly & Co), and the like;
inhibitors of GLP-1 degradation (a.k.a., DPP-IV inhibitors), which may be orally administered drugs that improve glycemic control by preventing DPP-IV degradation of GLP-1 and GIP and increasing incretin hormone levels to restore beta cell mass or function, including, without limitation, members of the group consisting of Sitagliptin (a.k.a. MK-0431, Merck), Vildagliplin (a.k.a. LAF-237) and NVP DPP728 (both of Novartis), Saxagliptin (Bristol Myers Squibb), P32/98 (Probiodrug) and FE 999011 (a.k.a. [(2S)-1([2′S]-2′-amino-3′,3′dimethyl-butanoyl)-pyrrolidine-2-carbonitrile] developed by Ferring Research institute), PHX1149 (Phenomix), and the like;
gastric inhibitory polypeptide (GIP) and analogs thereof (e.g., which are disclosed in U.S. Patent Publication No. 20050233969),
peptides such as gastrin and/or epidermal growth factor 1, including islet neogenesis therapy (Transition Therapeutics),
insulin like growth factor 1 or 2;
Parathyroid hormone related peptide (PTHrP) and
Hepatocyte growth factor or islet neogenesis associated protein (INGAP).

Other exemplary methods of inducing β-cell differentiation or growth include, without limitation, providing one or any combination of transcription factors shown to be important for insulin gene transcription or β-cell growth or development, including, without limitation, members of the group consisting of Neurogen 3, PDX-1, NKX6.1 and the like.

Other exemplary agents that induce pancreatic β-cell or insulin producing cell growth and/or differentiation include, but are not limited to, members of the group consisting of: histone deacetylose inhibitors (HDAC) such as NVP-LAQ824, TrichostatinA-O hydroxamate, suberanihohydroxamic or cyclic tetrapeptides, apicidin and trapoxin as well as synthetic inhibitors, including CG1521 and others, scriptide and analogs. Other HDAC inhibitors include: oxamflatin, pyroxamide, propenamides, chlamydocin, diheteropeptin, WF-3136, Cyl-1 and Cyl-2, FR 901228, cyclic-hydroxamic-acid-containing peptides, MS-275, CI-994 and depudecin.

Still other examples of exemplary agents that induce pancreatic β-cell or insulin producing cell growth and/or differentiation include, but are not limited to, amino-terminal extended forms of GLP-1 selected from the group consisting of: (a) glucagon-like peptide 1(7-37); (b) glucagon-like peptide 1(7-36) amide; and (c) an effective fragment or analog of (a) or (b) (each of which are described in U.S. Pat. No. 6,899,883 and U.S. Pat. No. 6,989,148).

In another exemplary embodiment, the invention involves the use of inventive pharmaceutical composition comprising an autoimmune blocker and a β-cell growth or differentiating agent to create or grow insulin producing cells in a test tube to be transplanted in patients by any acceptable procedure to prevent, treat or reverse T1DM or T2DM, in addition, this combined therapeutic approach can be given to a human to restore beta (insulin producing) cells in the body to prevent, treat or reverse T1DM, LADA or T2DM. Accordingly, in another exemplary embodiment, the invention provides a method for improving the outcome or success of cellular (islet cell, isolated β-cells, genetically engineered or induced (e.g. via transcription factors) β-cells) transplantation in a mammal to reverse T1DM, LADA and T2DM, comprising administering to the mammal (or cells to be transplanted) an effective amount of a pharmaceutical composition of the invention.

Exemplary LSF analogs include, without limitation, compounds, pharmaceutically acceptable derivatives (e.g., racemic mixtures, resolved enantiomers, diastereomers, tautomers, salts and solvates thereof) or prodrugs thereof, having the following Formula I:

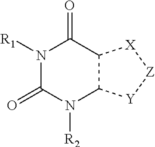

wherein:

the dashed lines, i.e., “ - - - ”, in Formula I represent a single or double bond;

X, Y and Z are independently selected from a member of the group consisting of C(R3), N, N(R3) and S;

R1 is selected from a member of the group consisting of hydrogen, methyl, a substituted alkyl (as defined herein, which, includes without limitation substituted C(5-9)alkyl), C(5-9)alkenyl, C(5-9 alkynyl, C(5-9)hydroxyalkyl, C(3-8)alkoxyl, C(59)alkoxyalkyl; and

R2 and R3 are independently selected from a member of the group consisting of hydrogen, halo, oxo, C(1-2)alkyl, C(1-20)hydroxyalkyl, C(1-20)thioalkyl, C(1-20)alkylamino, C(1-20)alkylaminoalkyl, (1-20)aminoalkyl, C(1-20)aminoalkoxyalkenyl, C(1-20)aminoalkoxyalkynyl, C(1-20)diaminoalkyl, C(1-20)triaminoalkyl, C(1-20)tetraaminoalkyl, C(5-15)aminotrialkoxyamino, C(1-0.20)alkylamido, C(1-20)alkylamidoalkyl, C(1-20)amidoalkyl, C(1-20)acetamidoalkyl, C(1-20)alkenyl, C(1-20)alkynyl, C(3-8)alkoxyl, C(1-11)alkoxyalkyl, and C(1-20)dialkoxyalkyl.

R1 is optionally substituted with a member selected from the group consisting of N—OH, acylamino, cyano (e.g., N—), cyanamido (e.g., NCNH—), cyanato (e.g., NCO—), sulfo, sulfonyl, sulfinyl, sulfhydryl (mercapto), sulfeno, sulfanilyl, sulfamyl, sulfamino, and phosphino, phosphinyl, phospho, phosphono and —NRaRb, wherein each of Ra and Rb may be the same or different and each is independently selected from the group consisting of hydrogen, optionally substituted alkyl, cycloalkyl, alkenyl, cycloalkenyl, alkynyl, aryl, heteroaryl and heterocyclic group.

Each R2 and R3 is optionally substituted with one or more members of the group consisting of hydroxyl, methyl, carboxyl, furyl, furfuryl, biotinyl, phenyl, naphthyl, amino group, amido group, carbamoyl group, cyano (e.g., NC—), cyanamido (e.g., NCNH—), cyanato (e.g., NCO—), sulfo, sulfonyl, sulfinyl, sulfhydryl (mercapto), sulfeno, sulfanilyl, sulfamyl, sulfamino, phosphino, phosphinyl, phospho, phosphono, N—OH, —Si(CH3)3 (a.k.a. SiMe3), C(1-3)alkyl, C(1-3)hydroxyalkyl, C(1-3)thioalkyl, C(1-3)alkylamino, benzyldihydrocinnamoyl group, benzoyldihydrocinnamido group, heterocyclic group and carbocyclic group.

The heterocyclic group or carbocyclic group is optionally substituted with one or more members of the group consisting of halo, hydroxyl, nitro (e.g., —NO2), SO2NH2, C(1-6)alkyl, C(1-6)haloalkyl, C(1-8)alkoxyl, C(1-11)alkoxyalkyl, C(1-6)alkylamino, and C(1-6)aminoalkyl.

Preferably, both X and Y are not N(R3) when Z is C(R3) and R3 is H or C(1-3)alkyl.

More preferably, R1 is not an ω-1 secondary alcohol substituted C(5-8) alkyl when both X and Y are N(R3), Z is C(R3) and R3 is H or C(1-3) alkyl.

In another exemplary aspect of the invention, R1 is an ω-1 secondary alcohol substituted C(5-8) alkyl when both X and Y are N(R3), Z is C(R3) and R3 is H or C(1-3) alkyl.

In a another aspect, more exemplary LSF analog compounds include the following compounds, pharmaceutically acceptable derivatives (e.g., racemic mixtures, resolved enantiomers, diastereomers, tautomers, salts and solvates thereof) or prodrugs thereof, having the following Formula II:

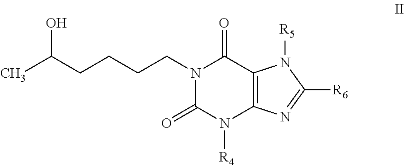

wherein R₄, R₅, and R₆ are independently selected from a member of the group consisting of hydrogen, halo, oxo, C₍₁₋₂₀₎alkyl, C₍₁₋₂₀₎hydroxyalkyl, C₍₁₋₂₀₎thioalkyl, C₍₁₋₂₀₎alkylamino, C₍₁₋₂₀₎alkylaminoalkyl, C₍₁₋₂₀₎aminoalkyl, C₍₁₋₂₀₎aminoalkoxyalkenyl, C₍₁₋₂₀₎aminoalkoxyalkynyl, C₍₁₋₂₀₎diaminoalkyl, C₍₁₋₂₀₎triaminoalkyl, C₍₁₋₂₀₎tetraaminoalkyl, C₍₃₋₁₅₎aminodialkoxyamino, C₍₅₋₁₅₎aminotrialkoxyamino, C₍₁₋₂₀₎alkylamido, C₍₁₋₂₀₎alkylamidoalkyl, C₍₁₋₂₀₎amidoalkyl, C₍₁₋₂₀₎acetamidoalkyl, C₍₁₋₂₀₎alkenyl, C₍₁₋₂₀₎alkynyl, C₍₃₋₈₎alkoxyl, C₍₁₋₁₁₎alkoxyalkyl, and C₍₁₋₂₀₎dialkoxyalkyl.

Each R4, R5 and R6 is optionally substituted with one or more members of the group consisting of hydroxyl, methyl, carboxyl, furyl, furfuryl, biotinyl, phenyl, naphthyl, amino group, amido group, carbamoyl group, cyano (e.g., NC—), cyanamido (e.g., NCNH—), cyanato (e.g., NCO—), sulfo, sulfonyl, sulfinyl, sulfhydryl (mercapto), sulfeno, sulfanilyl, sulfamyl, sulfamino, phosphino, phosphinyl, phospho, phosphono, N—OH, —Si(CH3)3, C(1-3)alkyl C(1-3)hydroxyalkyl, C(1-3)thioalkyl, C(1-3)alkylamino, benzyldihydrocinnamoyl group, benzoyldihydrocinnamido group, heterocyclic group and carbocyclic group.

The heterocyclic group or carbocyclic group is optionally substituted with one or more members of the group consisting of halo, hydroxyl, nitro (e.g., —NO2), SO2NH2, C(1-6) alkyl, C(1-6)haloalkyl C(1-8)alkoxyl, C(1-11)alkoxyalkyl, C(1-6)alkylamino, and C(1-6) aminoalkyl. In an exemplary embodiment, each R4, R5 and R6 are not simultaneously methyl.

In an exemplary embodiment, both R4 and R5 are not methyl when R6 is H.

In another exemplary embodiment, R6 is not methyl when R4 is methylfuryl and R5 is H.

In a further exemplary embodiment, R6 is not propyl or isopropyl when R4 is methyl and R5 is H.

In a still further exemplary embodiment, R4 is not acetamidohexyl when R5 is methyl and R6 is H.

Exemplary examples of R2, and R3 groups of Formula I and R4, R5 and R6 groups of Formula II include, without limitation, members selected from the group consisting of 1-adamantanemethyl, 1-phenylcyclopropyl, 1-phenylproply, 1-propenyl, 2-bromopropyl, 2-buten-2-yl, 2-butyl, 2-cyclohexylethyl, 2-cyclopentylethyl, 2-furyl, 2-hydroxyethyl, 2-hydroxystyryl, 2-methoxyethyl, 2-methoxystyryl, 2-methylbutyl, 2-methylcyclopropyl, 2-norboranemethyl, 2-phenylpropyl, 2-propenyl, 2-propyl, 2-thienyl, 2-trifluoromethylstyryl, 3,4,5-triethoxyphenyl, 3,4,5-trimethoxyphenyl, 3,4-dichlorobenzyl, 3,4-dichlorophenyl, 3,4-difluorophenyl, 3,4-difluorobenzyl, 3,4-dihydroxybenzyl, 3,4-dihydroxystyryl, 3,4-dimethoxybenzyl, 3,4-dimethoxyphenethyl, 3,4-dimethoxyphenyl, 3,4-dimethoxystyryl, 3,4-dimethylphenyl, 3,5-bis(trifluoromethyl)-benzyl, 3,5-dimethylphenyl, 3-bromo-4-methylphenyl, 3-bromobenzyl, 3-cyclohexylpropyl, 3-dimethylaminobutyl, 3-fluoro-4-methylphenyl, 3-fluorobenzyl, 3-hepten-3-yl, 3-hydroxy-n-butyl, 3-hydroxypropyl, 3-iodo-4-methylphenyl, 3-methoxy-4-methylphenyl, 3-methoxybenzyl, 3-methyl benzyl, 3-phenylpropyl, 3-trifluoromethylbenzyl, 4′-ethyl-4-biphenyl, 4-biphenyl, 4-bromobenzyl, 4-bromophenyl, 4-butylphenyl, 4-chloropentyl, 4-chlorostyryl, 4-ethoxybenzyl, 4-fluorobenzyl, 4-fluorophenyl, 4-hydroxyphenyl, 4-isobutylphenyl, 4-butylphenethyl, 4-isopropylphenyl, 4-methoxybenzyl, 4-methoxy-n-butyl, 4-methylbenzyl, 4-methylcyclohexanemethyl, 4-methylcyclohexyl, 4-phenylbenzyl, 4-t-butylcyclohexyl, 4-vinylphenyl, 5-hydroxyhexyl, alpha-methylstyryl, benzyl, cyclobutyl, cycloheptyl, cyclohexyl, cyclohexylmethyl cyclopentyl, ethyl, hexyl, isobutyl, isopropyl, isovaleryl, m-anisyl, methyl, m-tolyl, n-butyl, n-propyl, p-anisyl, phenethyl, phenyl, propyl, p-tolyl, styryl, t-butyl, and the like.

Exemplary R2, R3, R4, R5 and R6 groups include, without limitation, members selected from the group consisting of methyl, ethyl, oxo, isopropyl, n-propyl, isobutyl, n-butyl, t-butyl, 2-hydroxyethyl, 3-hydroxypropyl, 3-hydroxy-n-butyl, 2-methoxyethyl, 4-methoxy-n-butyl, 5-hydroxyhexyl, 2-bromopropyl, 3-dimethylaminobutyl, 4-chloropentyl, methylamino, aminomethyl, methylphenyl, and the like.

In accordance with the invention, the LSF analogs compounds, salts, solvates and prodrugs thereof, may exist in their tautomeric form (for example, as an amide or imino ether). All such tautomeric forms are contemplated herein as part of the invention. Further, all stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). Individual stereoisomers of the compounds described herein as suitable for use in the invention may, for example, be substantially free of other isomers, or may be admixed, for example, as racemates or with all other, or other selected, stereoisomers. The chiral centers of the invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, positional isomers, racemates or prodrugs of compounds disclosed herein.

In accordance with the principles of the invention, the LSF analogs described herein may contain one or more asymmetrically substituted carbon atoms and, thus, may occur as racemates and racemic mixtures, single enantiomers, diastereomeric mixtures and individual diastereomers. Each stereogenic carbon may be of the R or S configuration. Many geometric isomers of olefins, C—N double bonds, and the like can also be present in the compounds described herein, and all such stable isomers are contemplated in the invention. It is well known in the art how to prepare optically active forms, such as by resolution of racemic forms or by synthesis from optically active starting materials. All chiral, diastereomeric, racemic forms and all geometric forms of a structure are intended to be encompassed within the invention unless a specific stereochemistry or isomer form is specifically indicated.

The compounds of the invention may be modified by appending appropriate functionalites to enhance selective biological properties. Such modifications are known in the art and include, without limitation, those which increase penetration into a given biological compartment (e.g., blood, lymphatic system, central nervous system), increase oral or intravenous bioavailability, increase solubility to allow administration by injection, alter metabolism, alter rate of excretion, etc.

### DEFINITIONS

The following definitions are provided to assist the reader. Unless otherwise defined, all terms of art, notations and other scientific or medical terms or terminology used herein are intended to have the meanings commonly understood by those of skill in the art (e.g., biological, chemical, medical, etc.). In some cases, terms with commonly understood meanings are defined herein for clarity and/or for ready reference, and the inclusion of such definitions herein should not necessarily be construed to represent a substantial difference over the definition of the term as generally understood in the art.

“Stereoisomer” or “Optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds described herein as suitable for use in the invention which may give rise to stereoisomerism, the invention contemplates stereoisomers and mixtures thereof. The compounds described herein and their salts include asymmetric carbon atoms and may therefore exist as single stereoisomers, racemates, and as mixtures of enantiomers and diastereomers. Typically, such compounds will be prepared as a racemic mixture, if desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

“Enantiomers” means a pair of stereoisomers that are non-superimposable mirror images of each other.

“Diastereoisomers” or “Diastereomers” mean optical isomers which are not mirror images of each other.

“Racemic mixture” or “Racemate” mean a mixture containing equal parts of individual enantiomers.

“Non-Racemic Mixture” means a mixture containing unequal parts of individual enantiomers.

“Stable compound”, as used herein, is a compound that is sufficiently robust to survive isolation to a useful degree of purity from a reaction mixture, and formulation into an efficacious therapeutic agent, i.e., possesses stability that is sufficient to allow manufacture and that maintains the integrity of the compound for a sufficient period of time to be useful for the purposes detailed herein (e.g., therapeutic or prophylactic administration to a mammal or for use in affinity chromatography applications). Typically, such compounds are stable at a temperature of 40° C. or less, in the absence of moisture or other chemically reactive conditions, for at least a week. “Metabolically stable compound” denotes a compound that remains bioavailable when orally ingested by a mammal.

“Substituted”, as used herein, whether express or implied and whether preceded by “optionally” or not, means that any one or more hydrogen on the designated atom (C, N, etc.) is replaced with a selection from the indicated group, provided that the designated atom's normal valency is not exceeded, and that the substitution: results in a stable compound. For instance, when a CH2 is substituted by a keto substituent (═O), then 2 hydrogens on the atom are replaced. It should be noted that when a substituent is listed without indicating the atom via which such substituent is bonded, then such substituent may be bonded via any atom in such substituent. For example, when the substituent is piperazinyl, piperidinyl, or tetrazolyl, unless specified otherwise, said piperazinyl, piperidinyl, tetrazolyl group may be bonded to the rest of the compound of Formula I or II, as well as the R2, R3, R4, R5 and R6 groups substituted thereon, via any atom in such piperazinyl, piperidinyl, tetrazolyl group. Combinations of substituents and/or variables are permissible only if such combinations result in stable compounds. Further, when more than one position in a given structure may be substituted with a substituent selected from a specified group, the substituents may be either the same or different at every position. Typically, when a structure may be optionally substituted, 0-15 substitutions are preferred, 0-5 substitutions are more preferred, and 0-1 substitution is most preferred.

“Optional” or “optionally” means that the subsequently described event or circumstance may or may not occur, and that the description includes, without limitation, instances where said event or circumstance occurs and instances in which it does not. For example, optionally substituted alkyl means that alkyl may or may not be substituted by those groups enumerated in the definition of substituted alkyl.

“Acyl” denotes a radical provided by the residue after removal of hydroxyl from an organic acid. Examples of such acyl radicals include, without limitation, alkanoyl and aroyl radicals. Examples of such lower alkanoyl radicals include, without limitation, formyl, acetyl, propionyl, butyryl, isobutyryl, valeryl, isovaleryl, pivaloyl, hexanoyl, trifluoroacetyl.

“Acylamino” denotes an N-substituted amide, i.e., RC(O)—NH and RC(O)—NR′—. A non-limiting example is acetamido.

“Acyloxy” means 1 to about 4 carbon atoms. Exemplary examples include, without limitation, alkanoyloxy, benzoyloxy and the like.

“Alkyl” or “lower alkyl” is intended to include both branched and straight-chain saturated aliphatic hydrocarbon radicals/groups having the specified number of carbon atoms. In particular, “alkyl” refers to a monoradical branched or unbranched saturated hydrocarbon chain, preferably having from 1 to 40 carbon atoms, more preferably 1 to 10 carbon atoms, even more preferably 1 to 6 carbon atoms, such as methyl, ethyl, n-propyl, isopropyl, n-buty, secondary butyl, tert-butyl, n-hexyl, n-octyl, n-decyl, n-dodecyl, 2-ethyldodecyl, tetradecyl, and the like, unless otherwise indicated.

“Substituted alkyl” refers to an alkyl group as defined above having from 1 to 5 substituents selected, without limitation, from the group consisting of alkoxyl, substituted alkoxyl, cycloalkyl, substituted cycloalkyl, cycloalkenyl, substituted cycloalkenyl, acyl acylamino, acyloxy, aminoacyl, aminoacyloxyl, oxyaminoacyl, azido, cyano, halogen, hydroxyl, keto, thioketo, carboxyl, carboxylalkyl, thioaryloxyl, thioheteroaryloxyl, thioheterocyclooxyl, thiol, thioalkoxyl, substituted thioalkoxyl, aryl, aryloxyl, heteroaryl, heteroaryloxyl, heterocyclic, heterocyclooxyl, hydroxyamino, alkoxyamino, nitro, —SO-alkyl, —SO-aryl, —SO-heteroaryl, —SO2-alkyl, —SO2-aryl, —SO2-heteroaryl, and —NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic group.

“Alkylamino” denotes amino groups which have been substituted with one or two alkyl radicals. Exemplary are “lower N-alkylamino” radicals having alkyl portions having 1 to 6 carbon atoms. Exemplary lower alkylamino may be mono or dialkylamino such as N-methylamino, N-ethylamino, N,N-dimethylamino, N,N-diethylamino or the like.

“Alkylaminoalkyl” embraces radicals having one or more alkyl radicals attached to an aminoalkyl radical.

“Alkylaminocarbonyl” denotes an aminocarbonyl group which has been substituted with one or two alkyl radicals on the amino nitrogen atom. Exemplary are “N-alkylaminocarbonyl” “N,N-dialkylaminocarbonyl” radicals. More exemplary are “lower N-alkylaminocarbonyl” “lower N,N-dialkylaminocarbonyl” radicals with lower alkyl portions as defined above.

“Alkylcarbonyl”, “arylcarbonyl” and “aralkylcarbonyl” include radicals having alkyl, aryl and aralkyl radicals, as defined above, attached via an oxygen atom to a carbonyl radical. Examples of such radicals include, without limitation, substituted or unsubstituted methylcarbonyl, ethylcarbonyl, phenylcarbonyl and benzylcarbonyl.

“Alkylsulfinyl” embraces radicals containing a linear or branched alkyl radical, of one to ten carbon atoms, attached to a divalent —S(═O)— radical. More exemplary alkylsulfinyl radicals are “lower alkylsulfinyl” radicals having alkyl radicals of one to six carbon atoms. Examples of such lower alkylsulfinyl radicals include, without limitation, methylsulfinyl, ethylsulfinyl, butylsulfinyl and hexylsulfinyl.

“Alkylsulfonyl” embraces alkyl radicals attached to a sulfonyl radical, where alkyl is defined as above. More exemplary alkylsulfonyl radicals are “lower alkylsulfonyl” radicals having one to six carbon atoms. Examples of such lower alkylsulfonyl radicals include, without limitation, methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

“Alkylthio” embraces radicals containing a linear or branched alkyl radical, of one to about ten carbon atoms attached to a divalent sulfur atom. More exemplary alkylthio radicals are “lower alkylthio” radicals having alkyl radicals of one to six carbon atoms. Examples of such lower alkylthio radicals are methylthio, ethylthio, propylthio, butylthio and hexylthio.

“Alkylthioalkyl” embraces radicals containing an alkylthio radical attached through the divalent sulfur atom to an alkyl radical of one to about ten carbon atoms. More exemplary alkylthioalkyl radicals are “lower alkylthioalkyl” radicals having alkyl radicals of one to six carbon atoms. Examples of such lower alkylthioalkyl radicals include, without limitation, methylthiomethyl.

“Alkylene” refers to a diradical of a branched or unbranched saturated hydrocarbon chain, preferably having from 1 to 40 carbon atoms, more preferably 1 to 10 carbon atoms, even more preferably 1 to 6 carbon atoms. This term is exemplified by groups such as methylene (—CH2-), ethylene (—CH2CH2-), the propylene isomers (e.g. —CH2CHCH2- and —CH(CH3)CH2-), and the like.

“Substituted alkylene” refers to: (1) an alkylene group as defined above having from 1 to 5 substituents selected from a member of the group consisting of alkoxyl, substituted alkoxyl, cycloalkyl, substituted cycloalkyl, cycloalkenyl, substituted cycloalkenyl, acyl, acylamino, acyloxyl, aminoacyl, aminoacyloxyl, oxyacylamino, azido, cyano, halogen, hydroxyl, keto, thioketo, carboxyl, carboxylalkyl, thiol, thioalkoxyl, substituted thioalkoxyl, aryl aryloxyl, thioaryloxyl, heteroaryl, heteroaryloxyl, thioheteroaryloxy, heterocyclic, heterocyclooxyl, thioheterocyclooxyl, nitro, and —NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic. Additionally, such substituted alkylene groups include, without limitation, those where 2 substituents on the alkylene group are fused to form one or more cycloalkyl, substituted cycloalkyl, cycloalkenyl, substituted cycloalkenyl, aryl, heterocyclic or heteroaryl groups fused to the alkylene group; (2) an alkylene group as defined above that is interrupted by 1-20 atoms independently chosen from oxygen, sulfur and NRa, where Ra is chosen from hydrogen, optionally substituted alkyl, cycloalkyl, alkenyl, cycloalkenyl, alkenyl cycloalkenyl, alkynyl, aryl, heteroaryl and heterocyclic, or groups selected from carbonyl, carboxyester, carboxyamide and sulfonyl; or (3) an alkylene group as defined above that has both from 1 to 5 substituents as defined above and is also interrupted by 1 to 20 atoms as defined above. Examples of substituted alkylenes are chloromethylene (—CH(C1)-), aminoethylene (—CH(NH2)CH2-), 2-carboxypropylene isomers (—CH2CH(CO2H)CH2-), ethoxyethyl (—CH2CH2O—CH2CH2-), ethylmethylaminoethyl (—CH2CH2N(CH3)CH2CH2-), 1-ethoxy-2-(2-ethoxy-ethoxy)ethane (—CH2CH2O—CH2CH2-OCH2CH2-OCH2CH2-), and the like.

“Alkynyl” is intended to include hydrocarbon chains of either a straight or branched configuration and one or more triple carbon-carbon bonds which may occur in any stable point along the chain, such as ethynyl, propynyl and the like. For example, alkynyl refers to an unsaturated acyclic hydrocarbon radical in so much as it contains one or more triple bonds, such radicals containing about 2 to about 40 carbon atoms, preferably having from about 2 to about 10 carbon atoms and more preferably having 2 to about 6 carbon atoms. Non-limiting examples of exemplary alkynyl radicals include, ethynyl, propynyl, butyn-1-yl, butyn-2-yl, pentyn-1-yl, pentyn-2-yl, 3-methylbutyn-1-yl, hexyn-1-yl, hexyn-2-yl, hexyn-3-yl, 3,3-dimethylbutyn-1-yl radicals and the like.

“Alicyclic hydrocarbon” means a aliphatic radical in a ring with 3 to about 10 carbon atoms, and preferably from 3 to about 6 carbon atoms. Examples of exemplary alicyclic radicals include, without limitation, cyclopropyl, cyclopropylenyl, cyclobutyl, cyclopentyl, cyclohexyl, 2-cyclohexen-1-ylenyl, cyclohexenyl and the like.

“Alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More exemplary haloalkoxy radicals are “lower haloalkoxy” radicals having one to six carbon atoms and one or more halo radicals. Examples of such radicals include, without limitation, fluoromethoxy, chloromethoxy, trifluoromethoxy, trifluoromethoxy, fluoroethoxy and fluoropropoxy. Further, “alkoxycarbonyl” means a radical containing an alkoxy radical, as defined above, attached via an oxygen atom to a carbonyl radical. More exemplary are “lower alkoxycarbonyl” radicals with alkyl portions having 1 to 6 carbons. Examples of such lower alkoxycarbonyl (ester) radicals include, without limitation, substituted or unsubstituted methoxycarbonyl, ethoxycarbonyl, propoxycarbonyl, butoxycarbonyl and hexyloxycarbonyl.

“Aminoalkyl” embraces alkyl radicals substituted with amino radicals. More exemplary are “lower aminoalkyl” radicals. Examples of such radicals include, without limitation, aminomethyl, aminoethyl, and the like.

“Aminocarbonyl” denotes an amide group of the formula —C(═O)NH2.

“Aralkoxy” embraces aralkyl radicals attached through an oxygen atom to other radicals.

“Aralkoxyalkyl” embraces aralkoxy radicals attached through an oxygen atom to a an alkyl radical.

“Aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy.

“Aralkylamino” embraces aralkyl radicals attached through an nitrogen atom to other radicals.

“Aralkylthio” embraces aralkyl radicals attached to a sulfur atom.

“Aralkylthioalkyl” embraces aralkylthio radicals attached through a sulfur atom to an alkyl radical.

“Aromatic hydrocarbon radical” means 4 to about 16 carbon atoms, preferably 6 to about 12 carbon atoms, more preferably 6 to about 10 carbon atoms. Examples of exemplary aromatic hydrocarbon radicals include, without limitation, phenyl, naphthyl, and the like.

“Aroyl” embraces aryl radicals with a carbonyl radical as defined above. Examples of aroyl include, without limitation, benzoyl, naphthoyl, and the like and the aryl in said aroyl may be additionally substituted.

“Arylamino” denotes amino groups which have been substituted with one or two aryl radicals, such as N-phenylamino. Arylamino radicals may be further substituted on the aryl ring portion of the radical.

“Aryloxyalkyl” embraces radicals having an aryl radical attached to an alkyl radical through a divalent oxygen atom.

“Arylthioalkyl” embraces radicals having an aryl radical attached to an alkyl radical through a divalent sulfur atom.

“Carbonyl”, whether used alone or with other terms, such as “alkoxycarbonyl”, denotes —(C═O)—.

“Carboxy” or “carboxyl”, whether used alone or with other terms, such as “carboxyalkyl”, denotes —CO2H.

“Carboxyalkyl” embraces alkyl radicals substituted with a carboxy radical. More exemplary are “lower carboxyalkyl” which embrace lower alkyl radicals as defined above, and may be additionally substituted on the alkyl radical with halo. Examples of such lower carboxyalkyl radicals include, without limitation, carboxymethyl, carboxyethyl and carboxypropyl.

“Cycloalkenyl” embraces partially unsaturated carbocyclic radicals having three to twelve carbon atoms. More exemplary cycloalkenyl radicals are “lower cycloalkenyl” radicals having four to about eight carbon atoms. Examples of such radicals include, without limitation, cyclobutenyl, cyclopentenyl and cyclohexenyl.

“Cycloalkyl” embraces saturated carbocyclic radicals having three to twelve carbon atoms. More exemplary cycloalkyl radicals are “lower cycloalkyl” radicals having three to about eight carbon atoms. Examples of such radicals include, without limitation, cyclopropyl, cyclobutyl, cyclopentyl and cyclohexyl.

“Hydroxyalkyl” embraces linear or branched alkyl radicals having one to about twenty carbon atoms any one of which may be substituted with one or more hydroxyl radicals. Exemplary hydroxyalkyl radicals are “lower hydroxyalkyl” radicals having one to six carbon atoms and one or more hydroxyl radicals. Non-limiting examples of such radicals include hydroxymethyl, hydroxyethyl, hydroxypropyl, hydroxybutyl and hydroxyhexyl.

“Sulfamyl”, “aminosulfonyl” and “sulfonamidyl” denote NH2O2S—.

“Sulfonyl”, whether used alone or linked to other terms such as alkylsulfonyl, denotes respectively divalent radicals —SO2-.

“Alkenyl” is intended to include hydrocarbon chains of either a straight or branched configuration and one or more unsaturated carbon-carbon bonds which may occur in any stable point along the chain. For example, alkenyl refers to an unsaturated acyclic hydrocarbon radical in so much as it contains at least one double bond. Such radicals containing from about 2 to about 40 carbon atoms, preferably from about 2 to about 10 carbon atoms and more preferably about 2 to about 6 carbon atoms. Non-limiting examples of exemplary alkenyl radicals include propylenyl, buten-1-yl, isobutenyl, penten-1-yl, 2-2-methylbuten-1-yl, 3-methylbuten-1-yl, hexen-1-yl, hepten-1-yl, and octen-1-yl, and the like

“Alkoxyl” represents an alkyl group of indicated number of carbon atoms attached through an oxygen bridge. “Alkoxy” and “alkyloxy” embrace linear or branched oxy-containing radicals each having alkyl portions of one to about ten carbon atoms. More exemplary alkoxy radicals are “lower alkoxy” radicals having one to six carbon atoms. Examples of such radicals include, without limitation, methoxy, ethoxy, propoxy, butoxy and tert-butoxy.

“Aryl” refers to an unsaturated aromatic carbocyclic group of from 6 to 20 carbon atoms having a single ring (e.g., phenyl) or multiple condensed (fused) rings (e.g., naphthyl or anthryl). “aryl” embraces aromatic radicals such as phenyl, naphthyl, tetrahydronaphthyl, indane and biphenyl. Unless otherwise constrained by the definition for the aryl substituent, such aryl groups can optionally be substituted with from 1 to 5 substituents selected from a member of the group consisting of acyloxyl, hydroxyl, thiol, acyl, alkyl, alkoxyl, alkenyl, alkynyl, cycloalkyl, cycloalkenyl, substituted alkyl, substituted alkoxyl, substituted alkenyl, substituted alkynyl, substituted cycloalkyl, substituted cycloalkenyl, aminoacyl, acylamino, alkaryl, aryl, aryloxyl, azido, carboxyl, carboxylalkyl, cyano, halo, nitro, heteroaryl, heteroaryloxyl, heterocyclic, heterocyclooxyl, aminoacyloxyl, oxyacylamino, thioalkoxyl, substituted thioalkoxyl, thioaryloxyl, thioheteroaryloxyl, —SO-alkyl, —SO-substituted alkyl, —SO-aryl, —SO-heteroaryl, —SO2-alkyl, —SO2-substituted alkyl, —SO2-aryl, —CO2-heteroaryl, trihalomethyl, NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic. Exemplary aryl substituents include, without limitation, without limitation, alkyl, alkoxyl, halo, cyano, nitro, trihalomethyl, and thioalkoxy (i.e., —S-alkyl).

“N-arylaminoalkyl” and “N-aryl-N-alkyl-aminoalkyl” denote amino groups which have been substituted with one aryl radical or one aryl and one alkyl radical, respectively, and having the amino group attached to an alkyl radical. Examples of such radicals include, without limitation, N-phenylaminomethyl and N-phenyl-N-methylaminomethyl.

“Carbocycle” or “carbocyclic group” is intended to mean any stable 3 to 7 membered monocyclic or bicyclic or 7 to 14 membered bicyclic or tricyclic or an up to 26 membered polycyclic carbon ring, any of which may be saturated, partially unsaturated, or aromatic.

“Substituted carbocycle” or “substituted carbocyclic group” refers to carbocyclic groups having from 1 to 5 substituents selected from a member of the group consisting of alkoxyl, substituted alkoxyl, cycloalkyl, cycloalkenyl, substituted cycloalkenyl, acyl, acylamino, acyloxyl, amino, aminoacyl, aminoacyloxyl, oxyaminoacyl, azido, cyano, halogen, hydroxyl, keto, thioketo, carboxyl, carboxyalkyl, thioaryloxyl, thioheteroaryloxyl, thioheterocyclooxyl, thiol, thioalkoxyl, substituted thioalkoxyl, aryl, aryloxy, heteroaryl, heteroaryloxyl, heterocyclic, heterocyclooxyl, hydroxyamino, alkoxyamino, nitro, —SO-alkyl, —SO-substituted alkyl, —SO-aryl, —SO-heteroaryl, —SO2-alkyl, —SO2-substituted alkyl, —SO2-aryl, —SO2-heteroaryl, and NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic. Exemplary examples of carbocyclic groups include, without limitation, members selected from the group consisting of adamantyl, anthracenyl, benzamidyl, benzyl, bicyclo[2.2.1]heptanyl, bicyclo[2.2.1]hexanyl, bicyclo[2.2.22]octanyl, bicyclo[3.2.0]heptanyl, bicyclo[4.3.0]nonanyl, bicyclo[4.4.0]decanyl, biphenyl, biscyclooctyl, cyclobutanyl (cyclobutyl), cyclobutenyl, cycloheptanyl (cycloheptyl), cycloheptenyl, cyclohexanedionyl, cyclohexenyl, cyclohexyl, cyclooctanyl, cyclopentadienyl, cyclopentanedionyl, cyclopentenyl, cyclopentyl, cyclopropyl, decalinyl, 1,2-diphenylethanyl, indanyl, 1-indanonyl, indenyl, naphthyl, napthlalenyl, phenyl, resorcinolyl, stilbenzyl, tetrahydronaphthyl (tetralin), tetralinyl, tetratonyl, tricyclododecanyl, and the like.

“Cycloalkyl” is intended to include saturated ring groups, including mono-, bi- or poly-cyclic ring systems, such as, without limitation, cyclopropyl, cyclobutyl, cyclopentyl, cyclohexyl, cycloheptyl cyclooctyl, and adamantyl. “Bicycloalkyl” is intended to include saturated bicyclic ring groups such as, without limitation, [3.3.0]bicyclooctane. [4.3.0]bicyclononane, [4.4.0]bicyclodecane (decalin), [2.2.2]bicyclooctane, and so forth.

“Halo” or “halogen” as used herein refers to fluoro, chloro, bromo and iodo; and “counterion” is used to represent a small, negatively charged species such as chloride, bromide, hydroxide, acetate, sulfate and the like.

“Haloalkyl” is intended to include both branched and straight-chain saturated aliphatic hydrocarbon groups having the specified number of carbon atoms, substituted with 1 or more halogen. Haloalkyl embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals, “Lower haloalkyl” embraces radicals having 1-6 carbon atoms. Non-limiting examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

“Heterocycle” or “heterocyclic group” refers to a saturated or unsaturated group having a single ring, multiple condensed rings or multiple covalently joined rings, from 1 to 40 carbon atoms and from 1 to 10 hetero ring atoms, preferably 1 to 4 hetero ring atoms, selected from nitrogen, sulfur, phosphorus, and/or oxygen. Preferably, “heterocycle” or “heterocyclic group” means a stable 5 to 7 membered monocyclic or bicyclic or 7 to 10 membered bicyclic heterocyclic ring that may be saturated, partially unsaturated, or aromatic, and that comprises carbon atoms and from 1 to 4 heteroatoms independently selected from a member of the group consisting of nitrogen, oxygen and sulfur and wherein the nitrogen and sulfur heteroatoms are optionally be oxidized and the nitrogen heteroatom may optionally be quaternized, and including any bicyclic group in which any of the above-defined heterocyclic rings is fused to a benzene ring. The heterocyclic groups may be substituted on carbon or on a nitrogen, sulfur, phosphorus, and/or oxygen heteroatom so long as the resulting compound is stable. Unless otherwise constrained by the definition for the heterocyclic substituent, such heterocyclic groups can be optionally substituted with 1 to 5, and preferably 1 to 3 substituents. Suitable, but non-limiting, examples of such substituents include members selected from the group consisting of alkoxyl, substituted alkoxyl, cycloalkyl, substituted cycloalkyl, cycloalkenyl, substituted cycloalkenyl, acyl, acylamino, acyloxyl, aminoacyl, aminoacyloxyl, oxyaminoacyl, cyano, halogen, hydroxy, keto, thioketo, carboxyl, carboxylalkyl, thioaryloxyl, thioheteroaryloxy, thioheterocycooxyl, thiol, thioalkoxyl, substituted thioalkoxy, aryl, aryloxyl, heteroaryl, heteroaryloxyl, heterocyclic, heterocyclooxyl, hydroxyamino, alkoxyamino, nitro, —SO-alkyl, —SO-substituted alkyl, —O-aryl, —SO-heteroaryl, —SO2-alkyl, —SO2-substituted alkyl, —SO2-aryl, —SO4-heteroaryl, and NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic.

Exemplary examples of such heterocylic groups include, without imitation, acridinyl, acridonyl, adeninyl, alkylpyridinyl, alloxanyl, alloxazinyl, anthracenyl, anthracenyl, anthranilyl, anthraquinonyl, anthrenyl, ascorbyl, azaazulenyl, azabenzanthracenyl, azabenzanthrenyl, azabenzonaphthenyl, azabenzophenansthrenyl, azachrysenyl, azacyclazinyl, azaindolyl, azanaphthacenyl, azanaphthalenyl, azphenoxazinyl, azapinyl, azapurinyl, azapyrenyl, azatriphenylenyl, azepinyl, azetidinedionyl, azetidinonyl, azetidinyl, azinondolyl, azinopyrrolyl, azinyl, aziridinonyl, aziridinyl, azirnyl, azocinyl, azoloaziny, azolyl, barbituric acid, benzacridinyl, benzazapinyl, benzazinyl, benzimidazolethionyl, benzimidazolonyl, benzimtdazolyl, benzisothazoyl, benzisoxazolyl, benzocnnolinyl, benzodiazocinyl, benzodioxanyl, benzodioxolanyl, benzodioxolyl, benzofuranyl (benzofuryl), benzofuroxanyl, benzonaphthyridinyl, benzopyranonyl (benzopyranyl), benzopyridazinyl, benzopyronyl, benzoquinlinyl, benzoquinolizinyl, benzothiadiazinyl, benzohiazepinyl, benzothiazinyl, benzothiazolyl, benrzothiepinyl, benzothiophenyl, benzotriazepinonyl, benzotriazoiyl, benzoxadizinyl, benzoxazinyl, benzoxazolinonyl, benzoxazolyl, benzylisoquinolinyl, beta-carbolinyl, biotinyl, bipyridinyl, butenolidyl, butyrolactonyl, caprolactamyl, carbazolyl, 4a H-carbazolyl, carbolinyl, catechinyl, chromanyl, chromenopyronyl, chromonopyranyl, chromylenyl, cinnolinyl, coumarinyl, coumaronyl, decahydroquinolinyl, decahydroquinolinyl, depsidinyl, diazaanthracenyl, diazaphenanthrenyl, diazepinyl, diazinyl, diaziridinonyl, diaziridinyl, diazirinyl, diazocinyl, dibenzazepinyl, dibenzofuranyl, dibenzothiophenyl, dibernzoxazepinyl, dichromylenyl dihydrobenzimidazolyl, dihydrobenzothiazinyl, dihydrofuranyl, dihydroisocoumarinyl, dihydroisoquinolinyl, dihydrooxazolyl, dihydropyranyl, dihydropyridazinyl, dihydropyridinyl, dihydropyridonyl, dihydropyrimidnyl, dihydropyronyl, dihydrothiazinyl, dihydrothiopyranyl, dihydroxybenzenyl, dimethoxybenzenyl, dilmethyxanthinyl, dioxadiazinyl, dioxanthylenyl, dioxanyl, dioxenyl, dioxepinyl, dioxetanyl, dioxinonyl, dioxinonyl, dioxiranyl, dioxolanyl, dioxolonyl, dioxolyl, dioxopiperazinyl, diprylenyl, dipyrimidopyrazinyl, dithiadazolyl, dithiazolyl, 2H,6H-1,5,2-dithiazinyl, dithietanyl, dithiolanyl, dithiolenyl, dithiolyl, enantholactamyl, episulfonyl, flavanyl, flavanyl, flavinyl, flavonyl, tfuoranyl, fluorescienyl, furandionyl, furanochronanyl, furanonyl, furanoquinolinyl, furanyl (furyl), furazanryl, furfuryl, furopyranyl, furopyrimidinyl, furopyronyl, furoxanyl, glutarimridyl, glycocyamidinyl, guaninyl, heteroazulenyl, hexahydropyrazinoisoquinolinyl, hexahydropyridazinyl, homophthalimidyl, hydantoinyl, hydrofuranyl, hydrofurmanonyl, hydroimidazolyl, hydroindolyl, hydropyranyl, hydropyrazinyl, hydropyrazolyl, hydropyridazinyl, hydropyridinyl, hydropyrimidinyl, hydropyrrolyl, hydroquinolinyl, hydrothiochrromenyl, hydrothiophenyl, hydrotriazolyl, hydroxytrizinyl, imidazolethionyl, imidazolidinyl, imidazolinyl, imidazolonyl, imidazolyl, imidazoquinazolinyl, imidazothiazolyl, indazolebenzopyrazolyl, indazolyl, 1H-indazolyl, indenyl, indolinyl, indolizinyl, indolizinyl, indolonyl, indolyl, 3H-indolyl, indoxazenyl, inosinyl, isatinyl, isatogenyl, isoalloxazinyl, isobenzofurandionyl, isobenzofuranyl, isochromanyl, isoflavonyl, isoindolinyl (isoindolyl), isoindolobenzazepinyl, isoquinolinyl, isoquinuclidinyl, isothiazoyl, isoxazolidinyl, isoxazolinonyl, isoxazolinyl, isoxazolonyl, isoxazolyl, lactamyl, lactonyl, lumazinyl, maleimidyl, methlbenzamidyl, methylbenzoyleneureayl, methyldihydrouracilyl, methidioxotetrahydropteridinyl, methylputinyl, methylyhyminyl, methylthyminyl, methyluracilyl, methylxanthinyl, monoazabenzonaphthenyl, morpholinyl (morpholino), napthacenyl, naphthalenyl, naphthimidazolyl, naphthimidazopyridinedinyl, naphthindolizinedionyl, octahydroioquinoinyl, octylcartboxamidobenzenyl, oroticyl, oxadiazinyl, oxadiazolyl, oxathianyl, oxathiazinonyl, oxathietanyl, oxathiiranyl, oxathiolanyl, oxatriaolyl, oxazinonyl, oxaziranyl, oxazirdinyl, oxazolidinonyl, oxazolidinyl, oxazolidonyl, oxazolinonyl, oxazolinyl, oxazolonyl, oxazolopyrimidinyl, oxazolyl, oxepinyl, oxetananonyl, oxetanonyl, oxetanyl, oxindolyl, oxiranyl, oxolenyl, pentazinyl, pentazolyl, perhydroazolopyridinyl, perhydrocinnolinyl, perhydroindolyl, perhydropyrroloazinyl, perhydrrpyrrolooxazinyl, perhydropyrrolothiazinyl, pehydrothiazinonyl, perimidinyl, petrazinyl, phenanthraquinonyl, phenanthridinyl, phenoxazonyl, phenarsazinyl, phenazinyl, phenothiazinyl, phenoxanthinyl, phenoxazinyl, phenoxazonyl, phthalazinyl, phthalideisoquinolinyl, phthalimidyl, phthalonyl, piperazindionyl, piperazinodionyl, piperaznyl, piperidinyl, piperidonyl, 4-piperidonyl, polyoxadiazoyl, polyquinoxalinyl, prolinyl, prylenyl, pteridinyl, pterinyl, purinyl, pyradinyl, pyranoazinryl, pyranoazolyl, pyranonyl, pyranopyradinyl, pyranopyrandionyl, pyranopyridinyl, pyranoquionlinyl, pyranyl, pyrazinyl, pyrazolidnyl, pyrazolidonyl, pyrazolinonyl, pyrazolinyl, pyrazolobenzodiazepinyl, pyrazolonyl, pyrazolospyridinyl, pyrazolopyrimidinyl, pyrazolortriazinyl, pyrazolyl, pyrenyl, pyridazinyl, pyridazonyl, pyridinethionyl, pyridinonaphthalenyl, pyrdiropyridinyl, pyridocolinyl, pyridoindolyl, pyridopyrazinyl, pyridopyridinyl, pyridopyrimidinyl, pyridopyrrolyl, pyridoquinrolinyl, pyridyl (pyridinyl), pyrmidinethionyl, pyrimidinyl, pyrimidionyl, pyrimidoazepinyl, pyrimidopteridinyl, pyrrolizinyl, pyrrocolinyl, pyrrolidnyl, 2-pyrrolidinyl, pyrrolinyl, pyrrolizidinyl, pyrrolizinyl, pyrrolobenzodiazepinyl, pyrrolodiazinyl, pyrrolonyl, pyrrolopyriamidinyl, pyrroloquinolonyl, pyrrolyl, 2H-pyrrolyl, quinacridonyl, quinazolidinyl, quinazolinonyl, quinazolinyl, quinolinyl, quinolizidinyl, quinolizinyl, 4H-quinolizinyl, quinolonyl, quinonyl, quinazolinyl, quinolinyl, quinuclidinyl, rhrodaminyl, spirocoumaranyl, succinimidyl, sulfolanyl, sulfolenyl, sutamyl, sultinyl, sultonyl, sydononyl, tetrahydrofuranyl, tetrahydroisoquinolinyl, tetrahydrooxazolyl, tetrahydropyranyl, tetrahydropyrazinyl, tetrahydropyridazinyl, tetrahydropyridinyl, tetrahydroquinolinyl, tetrahydroquinoxalinyl, tetrahydrothiapyranyl, tetrahydrothiazolyl, tetrahydrothiophenyl, tetrahydrothiopyranonyl, tetrahydrothiopyranyl, tetraoxanyl, tetrazepinyl, tetrazinyl, tetrazolyl, tetronyl, thiabenzenyl, thiachromanyl, thiadecalinyl, thiadiazinyl, 6H-1,2,5-thiadiazinyl, thiadiazolinyl, thiadiazolyl, thiadioxazinyl, thianaphthenyl, thianthrenyl, thiapyranyl, thiapyronyl, thiatriazinyl, thiatriazolyl, thiazepinyl, thiazetidinyl, thiazinyl, thiaziridinyl, thiazolidinonyl, thiazolidinyl, thiazolinonyl, thiazolinyl, thiazolobenzimidazolyl, thiazolopyridinyl, thiazolyl, thienopryidinyl, thienopyrimidinyl, thienopyrrolyl, thienothiophenyl, thienyl, thiepinyl, thietanyl, thiiranyl, thiochromenyl, thiocoumarinyl, thiolanyl, thiolenyl, thiolyl, thiophenyl, thiopyranyl, thyminyl, triazzanthracenyl, triazepirnonyl, triazepinyl, triazinoindolyl, triazinyl, triazolinedionyl, triazolinyl, triazolopyridinyl, triazolopyrimidinyl, triazolyl, trioxanyl, triphenodioxazinyl, triphenodithiazinyl, trithdiadaepinyl, trithianyl, trxolanyl, trizinyl, tropanyl, uracilyl, xacthenyl, xanthinyl, xanthony, xanthydrolyl, xylitolyl, and the like as well N-alkoxy-nitrogen containing heterocycles. Exemplary heterocyclic groups include, without limitation, members of the group consisting of acridinyl, azirinyl, azocinyl, azepinyl, banzimidazoyl, benzodioxolanyl, benzofuranyl, benzothiophenyl, carbazole, 4a H-carbazole, chromanyl, chromenyl, cinnolinyl, decahydroquinolinyl, dioxoindolyl, furazanyl, furyl, furfuryl, imidazolidinyl, imidazolinyl, imidazolyl, 1H-indazolyl, indolenyl, indolinyl, indolizinyl, indolyl, 3H-indolyl, isobenzofuranyl, isochromanyl, isoindolinyl, isoindolyl, isoquinolinyl, isothiazolyl, isoxazoyl, morpholinyl, naphthalenyl, naphthyridinyl, norbornanyl, norpinanyl, octahydroisoquinolinyl, oxazolidinyl, oxazolyl, oxiranyl, perimidinyl, phenanthridinyl, phenanthrolinyl, phenarsazinyl, phenazinyl, phenothiazinyl, phenoxathiinyl, phenoxazinyl, phenyl, phthalazinyl, piperazinyl, piperidinyl, 4-piperidonyl, piperidyl, pteridinyl, purinyl, pyranyl, pyrazinyl, pyrazolidinyl, pyrazolinyl, pyrazolyl, pyrenyl, pyridazinyl, pyridinyl, pyridyl, pyridyl, pyrimidinyl, pyrrolidinyl, 2-pyrrolidonyl, pyrrolonyl, pyrrolyl, 2H-pyrrolyl, quinazolinyl, 4H-quinaolizinyl, quinolinyl, quinoxalinyl, quinuclidinyl, β-carbolinyl, tetrahydrofuranyl, tetrahydroisoquinolinyl, tetrahydroquinolinyl, tetrazolyl, 6H-1,2,5-thiadiazinyl, 2H-,6H-1,5,2-dithiazinyl, thianthrenyl, thiazolyl, thienyl, thiophenyl, triazinyl, xanthenyl, xanthinyl, and the like.

“Pharmaceutically acceptable derivative” or “prodrug” means any pharmaceutically acceptable salt, ester, salt of an ester, or other derivative of a compound of the invention which, upon administration to a recipient, is capable of providing (directly or indirectly) a compound of this invention. The term “prodrug”, as employed herein, denotes a compound that is a drug precursor which, upon administration to a subject, undergoes chemical conversion by metabolic or chemical processes to yield a pharmaceutically active compound. Particularly favored derivatives and prodrugs are those that increase the bioavailability of the compounds of this invention when such compounds are administered to a mammal (e.g., by allowing an orally administered compound to be more readily absorbed into the blood) or that enhance delivery of the parent compound to a biological compartment (e.g., the brain or lymphatic system) relative to the parent species. Prodrugs are considered to be any covalently bonded carriers which release the active parent drug according to Formula I or II in vivo when such prodrug is administered to a mammalian subject. Exemplary prodrugs include, without limitation, derivatives where a group that enhances aqueous solubility or active transport through the gut membrane is appended to the structure of Formula I or II. Prodrugs of the compounds of Formula I or II are prepared by modifying functional groups present in the compounds in such a way that the modifications are cleaved, either in routine manipulation or in vivo, to the parent compounds. Prodrugs include compounds of Formula I or it wherein hydroxy, amino, sulfhydryl, or carboxyl groups are bonded to any group that, when administered to a mammalian subject, cleaves to form a free hydroxyl, amino, sulfhydryl, or carboxyl group, respectively. Examples of prodrugs include, but are not limited to, acetate, formate and benzoate derivatives of alcohol and amine functional groups in the compounds of Formula I or II, and the like. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference.

“Solvate” means a physical association of a compound described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of exemplary solvates include ethanolates, methanolates, and the like. “Hydrate” is a solvate wherein the solvent molecule is H₂O.

“Pharmaceutically acceptable salts” refer to derivatives of the disclosed compounds wherein the parent compound of Formula I or II is modified by making acid or base salts of the compound of Formula I or II. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds of Formula I or II include the conventional nontoxic salts or the quaternary ammonium salts of the compounds of Formula I or II formed, for example, from nontoxic inorganic or organic acids. For example, such conventional non-toxic salts include, without limitation, those derived from inorganic acids such as acetic, 2-acetoxybenzoic, adipic, alginic, ascorbic, aspartic, benzoic, benzenesulfonic, bisulfic, butyric, citric, camphoric, camphorsulfonic, cyclopentanepropionic, digluconic, dodecylsulfanilic, ethane disulfonic, ethanesulfonilic, fumaric, glucoheptanoic, glutamic, glycerophosphic, glycolic, hemisufanoic, heptanoic, hexanoic, hydrochloric, hydrobromic, hydroiodic, 2-hydroxyethanesulfonoic, hydroxymaleic, isethionic, lactic, malic, maleic, methanesulfonic, 2-naphthalenesulfonilic, nicotinic, nitric, oxalic, palmic, pamoic, pectinic, persulfanilic, phenylacetic, phosphoric, propionic, pivalic, propionate, salicylic, succinic, stearic, sulfuric, sulfamic, sulfanilic, tartaric, thiocyanic toluenesulfonic, tosylic, undecanoatehydrochloric, and the like. The pharmaceutically acceptable salts of the invention can be synthesized from the compounds of Formula I or II which contain a basic or acidic moiety by conventional chemical methods, for example, by reacting the free base or acid with stoichiometric amounts of the appropriate base or acid, respectively, in water or in an organic solvent, or in a mixture of the two (nonaqueous media like ether, ethyl acetate, ethanol, isopropanol, or acetonitrile are preferred) or by reacting the free base or acid with an excess of the desired salt-forming inorganic or organic acid or base in a suitable solvent or various combinations of solvents. Lists of suitable salts are found in Remington's Pharmaceutical Sciences, 17th ed., Mack Publishing Company, Easton, Pa., 1985, p. 1418, et al., the entire disclosure of which is incorporated herein by reference.

Further, exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould. International J. of Pharmaceutics (1986) 33 201-217; Anderson et al. The Practice of Medicinal Chemistry (1996), Academic Press, New York; and in The Orange Book (Food & Drug Administration. Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others. All such acid salts and base salts are intended to be pharmaceutically acceptable salts within the scope of the invention and all acid and base salts are considered equivalent to the free forms of the corresponding compounds for purposes of the invention.

“Pharmaceutically effective” or “therapeutically effective” amount of a compound of the invention is an amount that is sufficient to effect the desired therapeutic, ameliorative, palliatory, eliminatory, inhibitory or preventative effect, as defined herein, when administered to a mammal in need of such treatment. The amount will vary depending upon the subject and disease condition being treated, the weight and age of the subject, the severity of the disease condition, the manner of administration and the like, which can be readily determined by one of skill in the art. The full therapeutic effect does not necessarily occur by administration of one dose and may occur only after administration of a series of doses. Thus, a therapeutically effective amount may be administered in one or more administrations.

“Mammal” means humans and other mammalian animals.

“Treatment” refers to any treatment of a disease (e.g., diabetes mellitus) or condition in a mammal, particularly a human, and includes, without limitation: (i) preventing the disease or condition from occurring in a subject which may be predisposed to the condition but has not yet been diagnosed with the condition and, accordingly, the treatment constitutes prophylactic treatment for the pathologic condition; (ii) inhibiting the disease or condition, i.e., arresting its development: (iii) relieving the disease or condition, i.e., causing regression of the disease or condition; or (iv) relieving the symptoms resulting from the disease or condition, e.g., relieving an inflammatory response without addressing the underlining disease or condition. “Treating” a condition or patient also may refer to taking steps to obtain beneficial or desired results, including clinical results. For purposes herein, beneficial or desired clinical results include, but are not limited to, alleviation or amelioration of one or more symptoms of diabetes, diminishment of extent of disease, delay or slowing of disease progression, amelioration, reduction, palliation or stabilization of the disease state, and other beneficial results described below.

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “manifestation” of a disease refers to a symptom, sign, anatomical state (e.g., lack of islet cells), physiological state (e.g., glucose level), or report (e.g., triglyceride level) characteristic of a subject with the disease.

As used herein, “reduction” of a symptom or symptoms (and grammatical equivalents of this phrase) means decreasing of the severity or frequency of the symptom(s), or elimination of the symptom(s).

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a prophylactically effective amount may be administered in one or more administrations.

As used herein, “TID”, “QD” and “QHS” have their ordinary meanings of “three times a day”, “once daily,” and “once before bedtime”, respectively.

The invention also envisions the quaternization of any basic nitrogen-containing groups of the compounds disclosed herein. The basic nitrogen can be quaternized with any agents known to those of ordinary skill in the art including, without limitation, lower alkyl halides, such as methyl, ethyl, propyl and butyl chlorides, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

Without being bound by the above general structural descriptions/definitions, exemplary compounds suitable as biological/immune response modifiers or anti-inflammatory agents that effectively block autoimmune response or cytokine formation in a mammal, include, but are not limited to the following compounds. It will be appreciated, as noted above, that where an R or S enantiomer is exemplified for each particular compound, the corresponding S or R enantiomer, respectively, is also intended even though it may not be specifically shown below.

More exemplary compounds of the invention having utility for inhibiting IL-12 signaling include without limitation, the following:

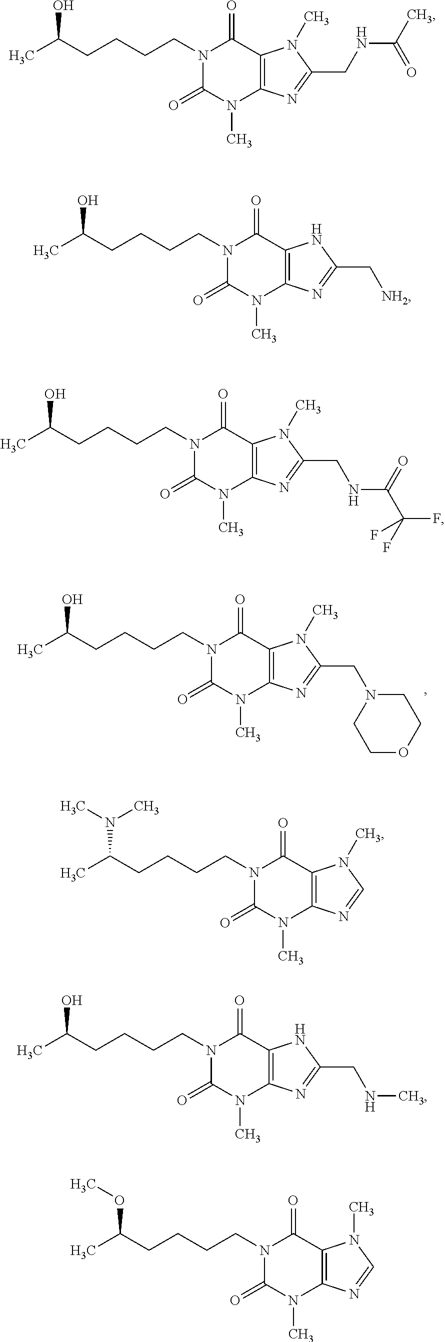

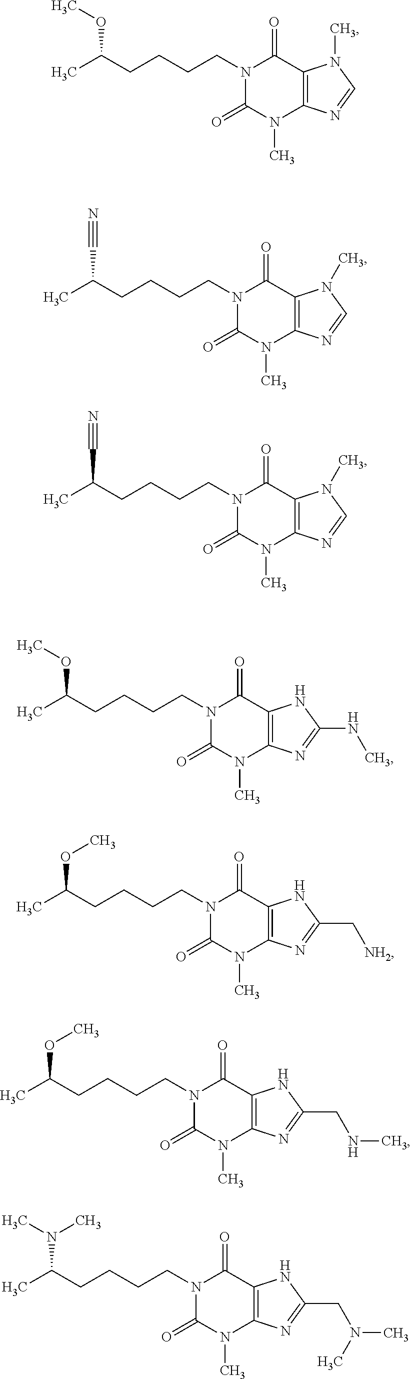

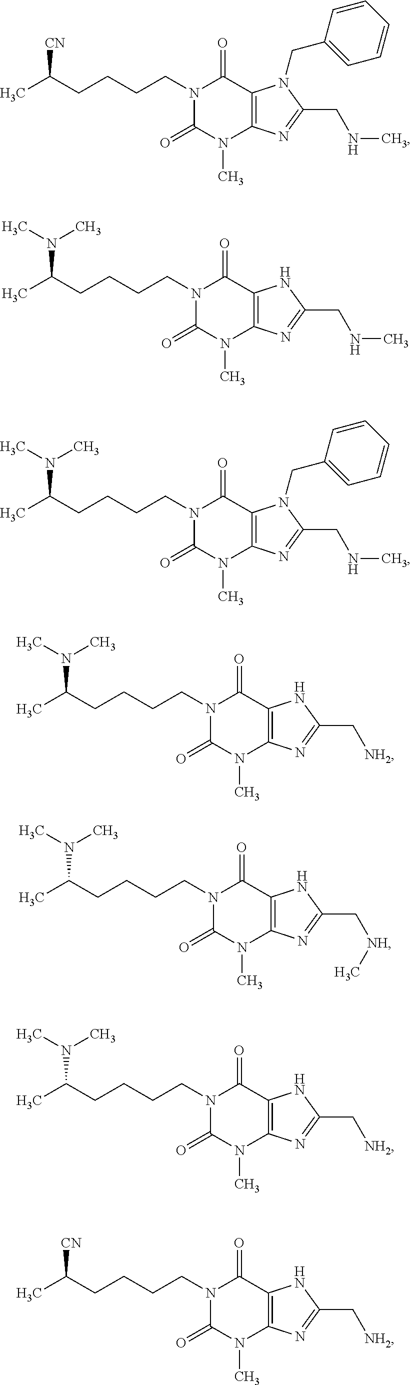

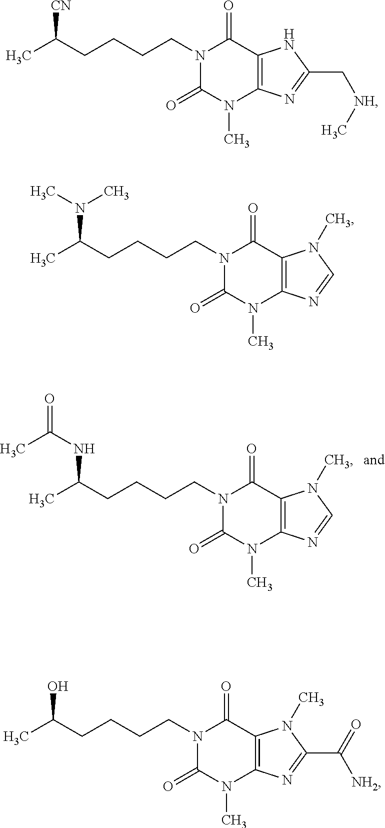 [SMILES: CNCc1nc2c([nH]1)c(=O)n(CCCC[C@@H](C)C#N)c(=O)n2C ; C[C@H](CCCCn1c(=O)c2c(ncn2C)n(C)c1=O)N(C)C ; CC(=O)N[C@H](C)CCCCn1c(=O)c2c(ncn2C)n(C)c1=O ; C[C@@H](O)CCCCn1c(=O)c2c(nc(C(N)=O)n2C)n(C)c1=O]

Further representative compounds of the invention having utility as a biological/immune response modifier (immunomodulating) or anti-inflammatory agent in accordance with the invention are set forth below in Table 1. The compounds in Table 1 have the following general structure of Formula II:

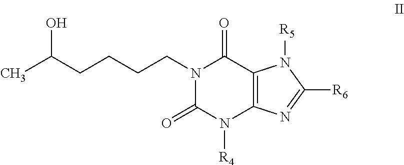

It is noted that in Table 1, “Me” represents “—CH3,” and “Et” represents “—CH2CH3.” n addition, although the below-exemplified moieties in Table 1 are representative of R4, R5 and R6 in Formula II, it will be understood that the exemplified moieties, without being limited by the above description/definitions, are also representative of R2 and R3 in Formula I.

TABLE 1

| R₄ | R₅ | R₆ |
|---|---|---|
| Me | H | 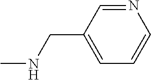 |
| Me | H | 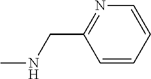 |
| Me | H | 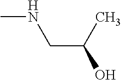 |
| Me | H | 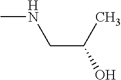 |
| Me | H | 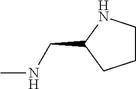 |
| Me | H | 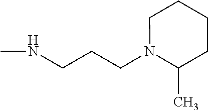 |
| Me | H | 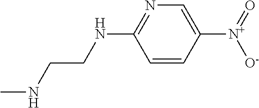 |
| Me | H | 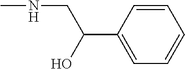 |
| Me | H | 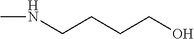 |
| Me | H | 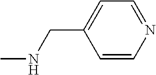 |
| Me | H | 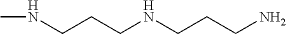 |
| Me | H | 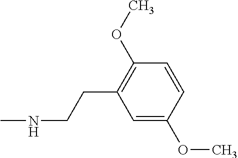 |
| Me | H | 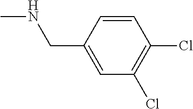 |
| Me | H | 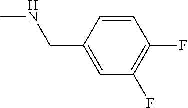 |
| Me | H | 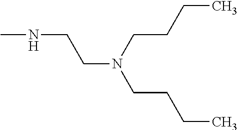 |
| Me | H | 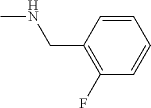 |
| Me | H | 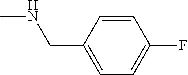 |
| Me | H | 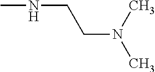 |
| Me | H | 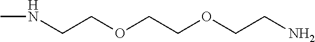 |
| Me | H | 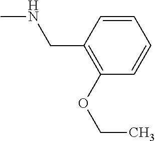 |
| Me | H | 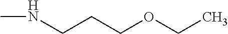 |
| Me | H | 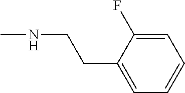 |
| Me | H | 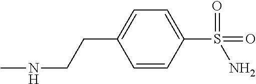 |
| Me | H | 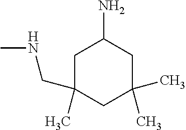 |
| Me | H | 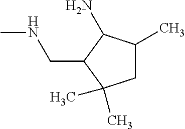 |
| Me | H | 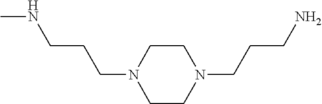 |
| Me | H | 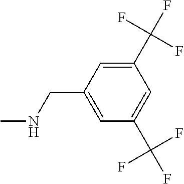 |
| Me | H | 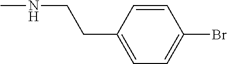 |
| Me | H | 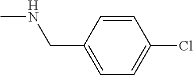 |
| Me | H | 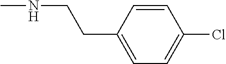 |
| Me | H | 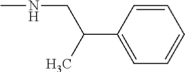 |
| Me | H | 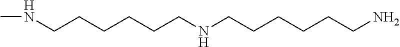 |
| Me | H | 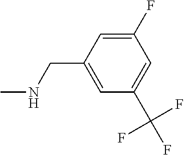 |
| Me | H | 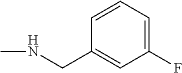 |
| Me | H | 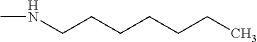 |
| Me | H | 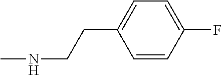 |
| Me | H | 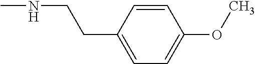 |
| Me | H | 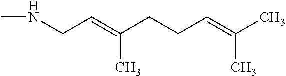 |
| Me | H | 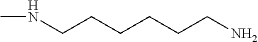 |
| Me | H | 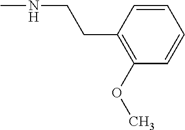 |
| Me | H | 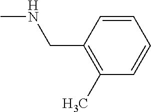 |
| Me | H | 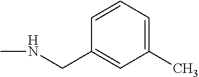 |
| Me | H | 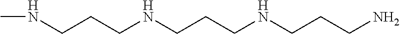 |
| Me | H | 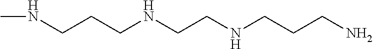 |
| Me | H | 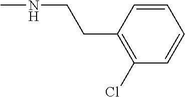 |
| Me | H | 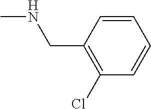 |
| Me | H | 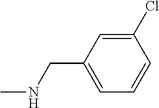 |
| Me | H | 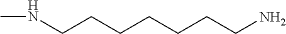 |
| Me | H | 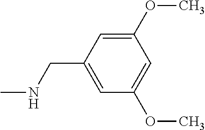 |
| Me | H | 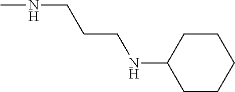 |
| Me | H | 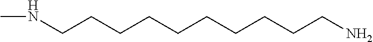 |
| Me | H | 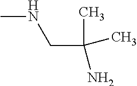 |
| Me | H | 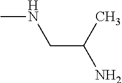 |
| Me | H | 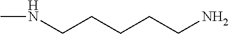 |
| Me | H | 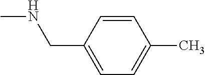 |
| Me | H | 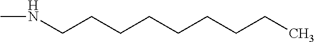 |
| Me | H | 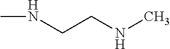 |
| Me | H | 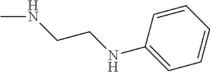 |
| Me | H | 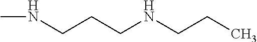 |
| Me | H | 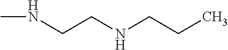 |
| Me | H | 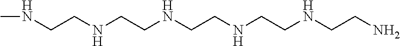 |
| Me | H | 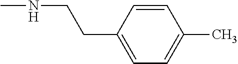 |
| Me | H | 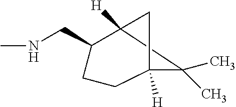 |
| Me | H | 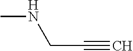 |
| Me | H | 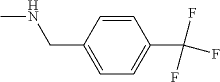 |
| Me | H | 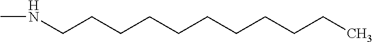 |
| Me | H | 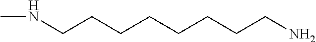 |
| Me | H | 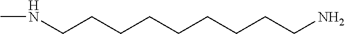 |
| Me | H | 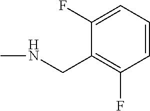 |
| Me | H | 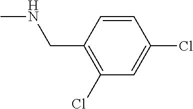 |
| Me | H | 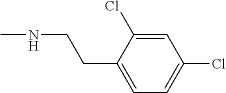 |
| Me | H | 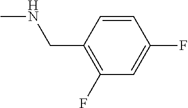 |
| Me | H | 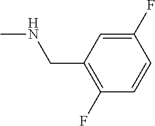 |
| Me | H | 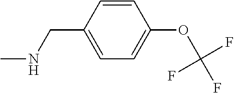 |
| Me | H | 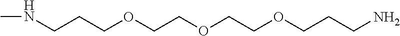 |
| Me | H | 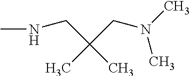 |
| Me | H | 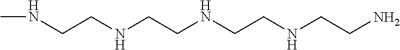 |
| Me | H | 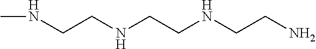 |
| Me | H | 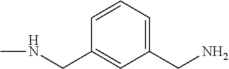 |
| Me | H | 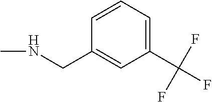 |
| Me | H | 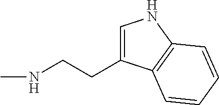 |
| Me | H | 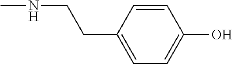 |
| Me | H | 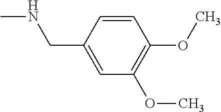 |
| Me | H | 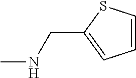 |
| Me | H | 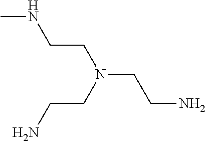 |
| Me | H | 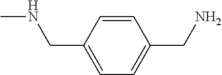 |
| Me | H | 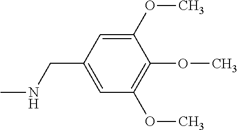 |
| Me | H | 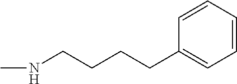 |
| Me | H | 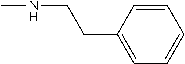 |
| Me | H | 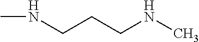 |
| Me | H | 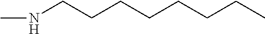 |
| Me | H | 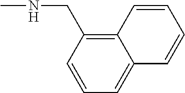 |
| Me | H | 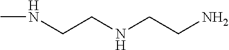 |
| Me | H | 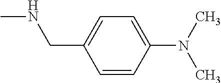 |
| Me | H | 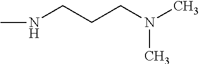 |
| Me | H | 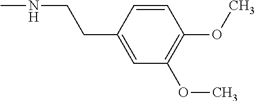 |
| Me | H | 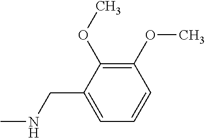 |
| Me | H | 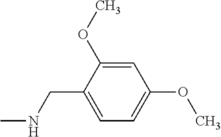 |
| Me | H | 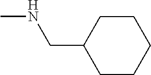 |
| Me | H | 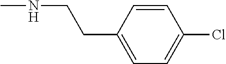 |
| Me | H | 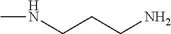 |
| Me | H | 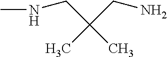 |
| Me | H | 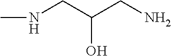 |
| Me | H | 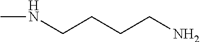 |
| Me | H | 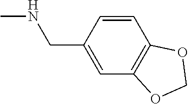 |
| Me | H | 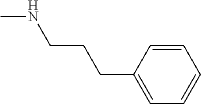 |
| Me | H | 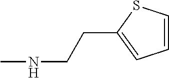 |
| Me | H | 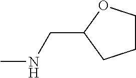 |
| Me | H | 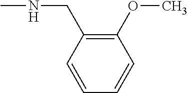 |
| Me | H | 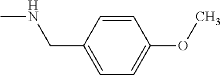 |
| Me | H | 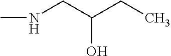 |
| Me | H | 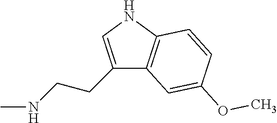 |
| Me | H | 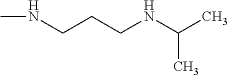 |
| Me | H | 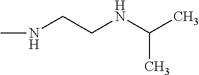 |
| Me | H | 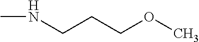 |
| Me | H | 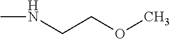 |
| Me | H | 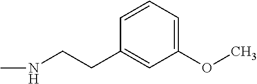 |
| Me | H | 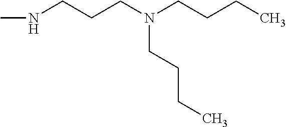 |
| Me | H | 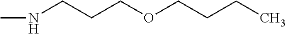 |
| Me | H | 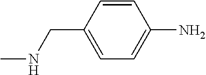 |
| Me | H | 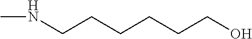 |
| Me | H | 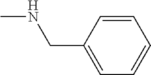 |
| Me | H | 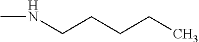 |
| Me | H | 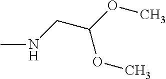 |
| Me | H | 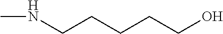 |
| Me | H | 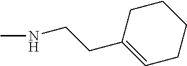 |
| Me | H | 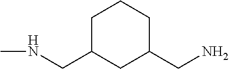 |
| Me | H | 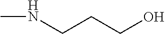 |
| Me | H | 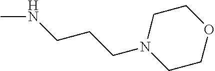 |
| Me | H | 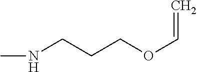 |
| Me | H | 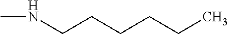 |
| Me | H | 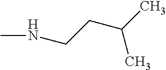 |
| Me | H | 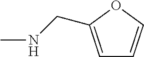 |
| Me | H | 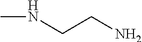 |
| Me | H | 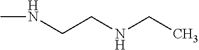 |
| Me | H | 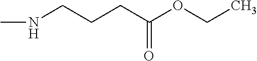 |
| Me | H | 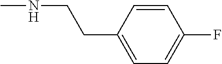 |
| Me | H | 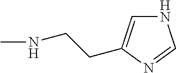 |
| Me | H | 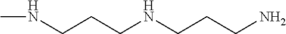 |
| Me | H | 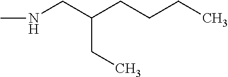 |
| Me | H | 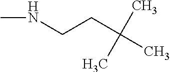 |
| Me | H | 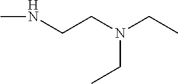 |
| Me | H | 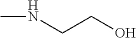 |
| Me | H | 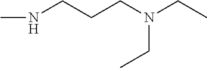 |
| Me | H | 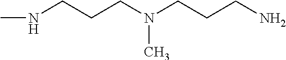 |
| Me | H | 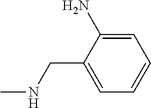 |
| Me | H | 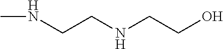 |
| Me | H | 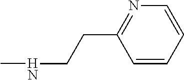 |
| Me | H | 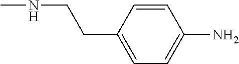 |
| Me | H | 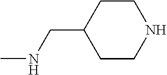 |
| Me | H | 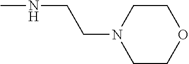 |
| Me | H | 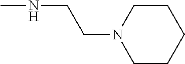 |
| Me | H | 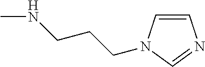 |
| Me | H | 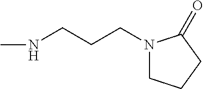 |
| Me | H | 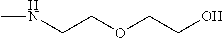 |
| Me | H | 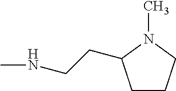 |
| Me | H | 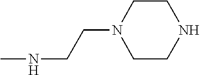 |
| Me | H | 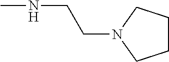 |
| Me | Me | 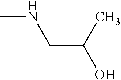 |
| Me | Me | 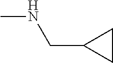 |
| Me | Me | 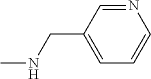 |
| Me | Me | 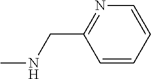 |
| Me | Me | 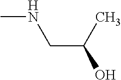 |
| Me | Me | 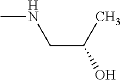 |
| Me | Me | 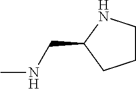 |
| Me | Me | 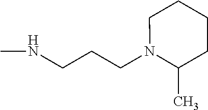 |
| Me | Me | 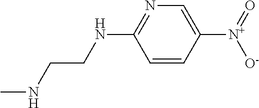 |
| Me | Me | 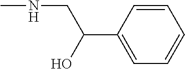 |
| Me | Me | 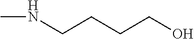 |
| Me | Me | 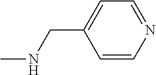 |
| Me | Me | 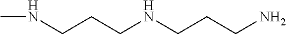 |
| Me | Me | 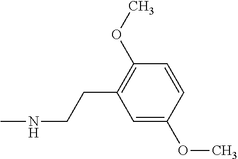 |
| Me | Me | 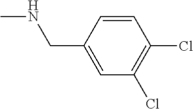 |
| Me | Me | 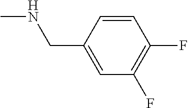 |
| Me | Me | 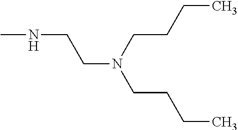 |
| Me | Me | 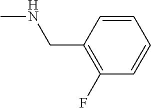 |
| Me | Me | 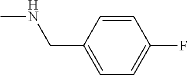 |
| Me | Me | 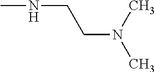 |
| Me | Me | 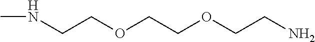 |
| Me | Me | 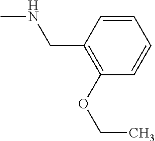 |
| Me | Me | 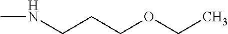 |
| Me | Me | 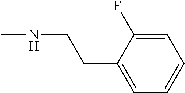 |
| Me | Me | 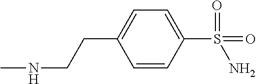 |
| Me | Me | 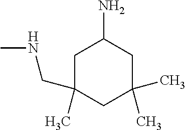 |
| Me | Me | 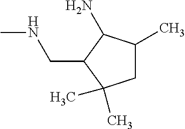 |
| Me | Me | 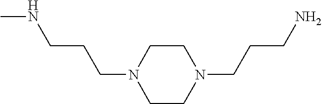 |
| Me | Me | 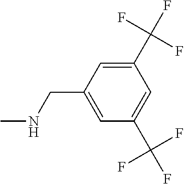 |
| Me | Me | 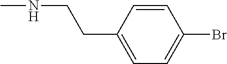 |
| Me | Me | 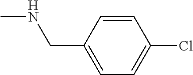 |
| Me | Me | 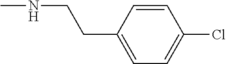 |
| Me | Me | 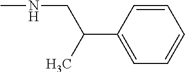 |
| Me | Me | 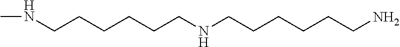 |
| Me | Me | 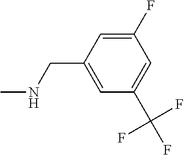 |
| Me | Me | 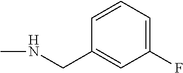 |
| Me | Me | 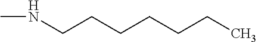 |
| Me | Me | 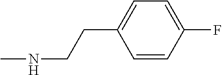 |
| Me | Me | 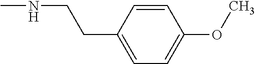 |
| Me | Me | 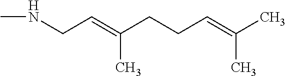 |
| Me | Me | 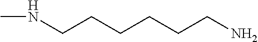 |
| Me | Me | 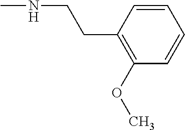 |
| Me | Me | 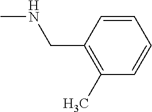 |
| Me | Me | 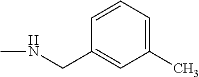 |
| Me | Me | 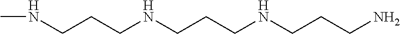 |
| Me | Me | 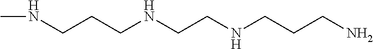 |
| Me | Me | 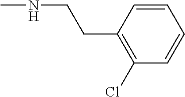 |
| Me | Me | 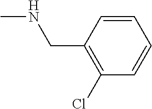 |
| Me | Me | 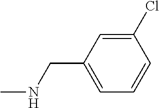 |
| Me | Me | 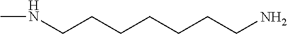 |
| Me | Me | 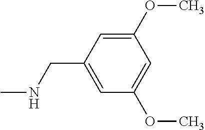 |
| Me | Me | 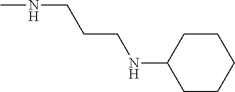 |
| Me | Me | 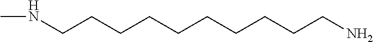 |
| Me | Me | 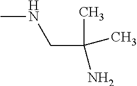 |
| Me | Me | 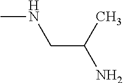 |
| Me | Me | 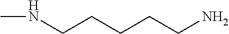 |
| Me | Me | 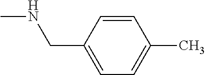 |
| Me | Me | 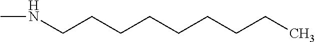 |
| Me | Me | 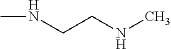 |
| Me | Me | 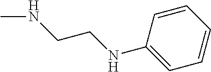 |
| Me | Me | 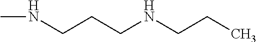 |
| Me | Me | 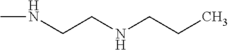 |
| Me | Me | 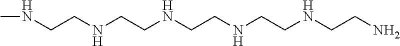 |
| Me | Me | 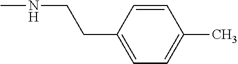 |
| Me | Me | 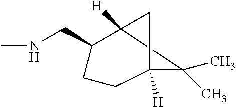 |
| Me | Me | 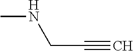 |
| Me | Me | 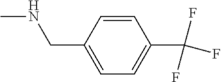 |
| Me | Me | 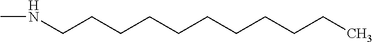 |
| Me | Me | 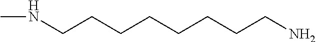 |
| Me | Me | 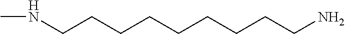 |
| Me | Me | 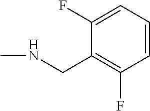 |
| Me | Me | 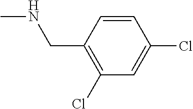 |
| Me | Me | 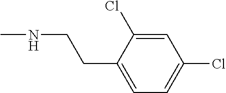 |
| Me | Me | 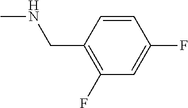 |
| Me | Me | 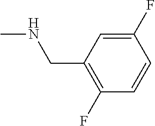 |
| Me | Me | 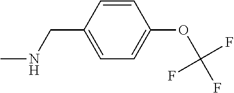 |
| Me | Me | 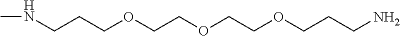 |
| Me | Me | 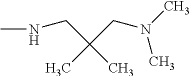 |
| Me | Me | 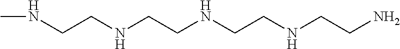 |
| Me | Me | 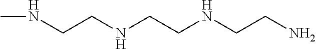 |
| Me | Me | 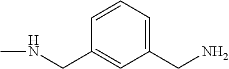 |
| Me | Me | 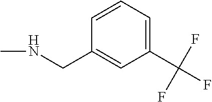 |
| Me | Me | 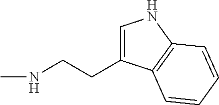 |
| Me | Me | 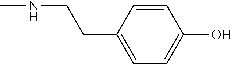 |
| Me | Me | 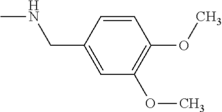 |
| Me | Me | 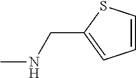 |
| Me | Me | 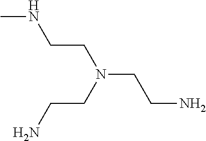 |
| Me | Me | 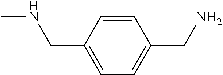 |
| Me | Me | 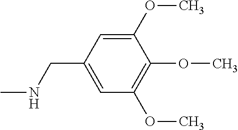 |
| Me | Me | 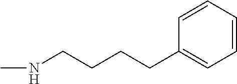 |
| Me | Me | 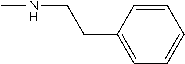 |
| Me | Me | 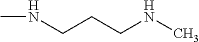 |
| Me | Me | 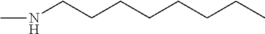 |
| Me | Me | 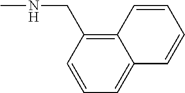 |
| Me | Me | 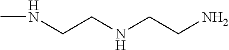 |
| Me | Me | 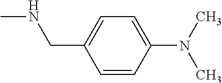 |
| Me | Me | 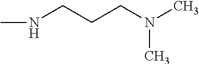 |
| Me | Me | 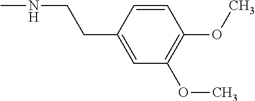 |
| Me | Me | 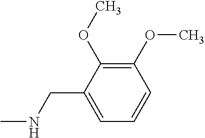 |
| Me | Me | 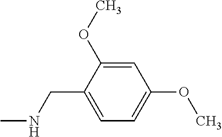 |
| Me | Me | 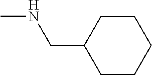 |
| Me | Me | 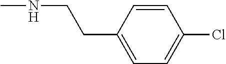 |
| Me | Me | 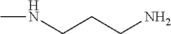 |
| Me | Me | 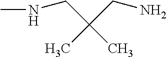 |
| Me | Me | 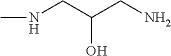 |
| Me | Me | 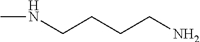 |
| Me | Me | 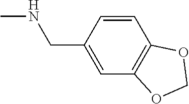 |
| Me | Me | 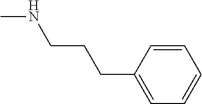 |
| Me | Me | 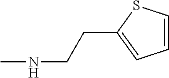 |
| Me | Me | 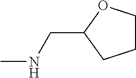 |
| Me | Me | 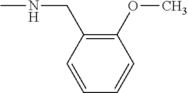 |
| Me | Me | 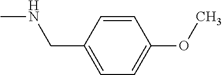 |
| Me | Me | 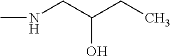 |
| Me | Me | 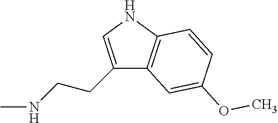 |
| Me | Me | 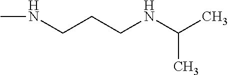 |
| Me | Me | 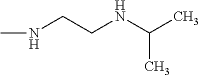 |
| Me | Me | 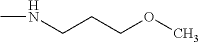 |
| Me | Me | 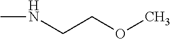 |
| Me | Me | 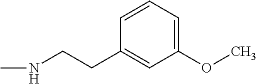 |
| Me | Me | 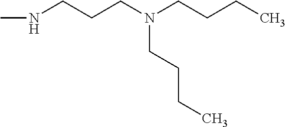 |
| Me | Me | 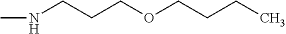 |
| Me | Me | 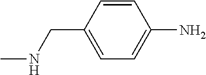 |
| Me | Me | 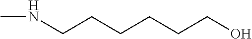 |
| Me | Me | 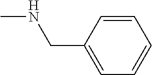 |
| Me | Me | 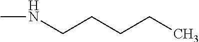 |
| Me | Me | 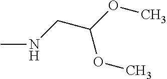 |
| Me | Me | 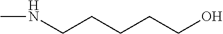 |
| Me | Me | 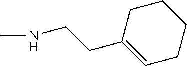 |
| Me | Me | 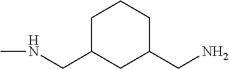 |
| Me | Me | 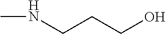 |
| Me | Me | 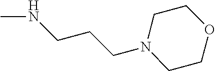 |
| Me | Me | 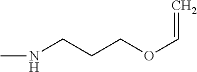 |
| Me | Me | 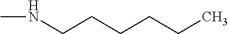 |
| Me | Me | 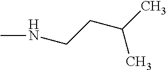 |
| Me | Me | 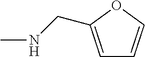 |
| Me | Me | 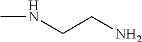 |
| Me | Me | 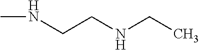 |
| Me | Me | 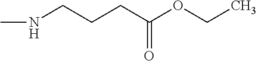 |
| Me | Me | 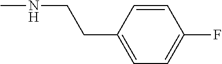 |
| Me | Me | 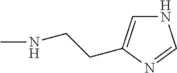 |
| Me | Me | 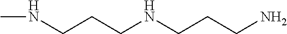 |
| Me | Me | 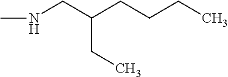 |
| Me | Me | 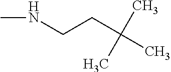 |
| Me | Me | 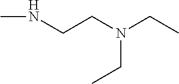 |
| Me | Me | 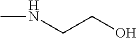 |
| Me | Me | 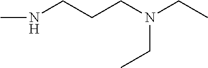 |
| Me | Me | 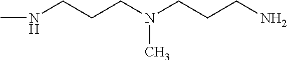 |
| Me | Me | 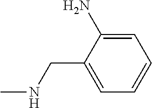 |
| Me | Me | 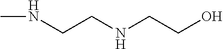 |
| Me | Me | 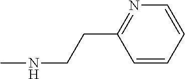 |
| Me | Me | 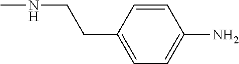 |
| Me | Me | 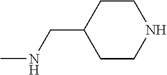 |
| Me | Me | 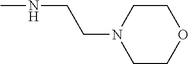 |
| Me | Me | 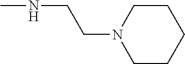 |
| Me | Me | 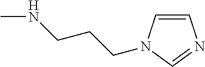 |
| Me | Me | 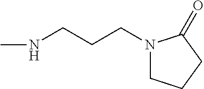 |
| Me | Me | 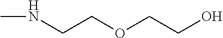 |
| Me | Me | 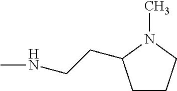 |
| Me | Me | 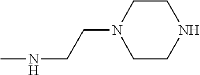 |
| Me | Me | 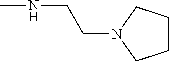 |
| Me | Me | 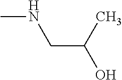 |
| Me | Me | 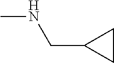 |
| Me | CH₂OEt | 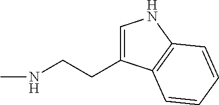 |
| Me | CH₂OEt | 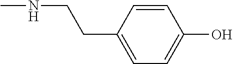 |
| Me | CH₂OEt | 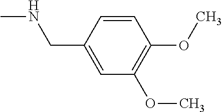 |
| Me | CH₂OEt | 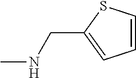 |
| Me | CH₂OEt | 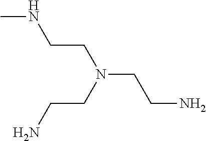 |
| Me | CH₂OEt | 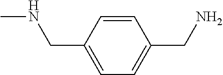 |
| Me | CH₂OEt | 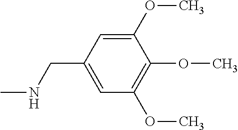 |
| Me | CH₂OEt | 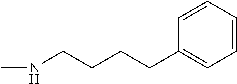 |
| Me | CH₂OEt | 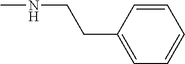 |
| Me | CH₂OEt | 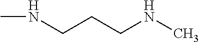 |
| Me | CH₂OEt | 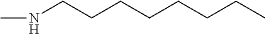 |
| Me | CH₂OEt | 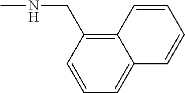 |
| Me | CH₂OEt | 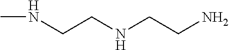 |
| Me | CH₂OEt | 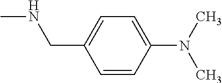 |
| Me | CH₂OEt | 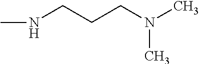 |
| Me | CH₂OEt | 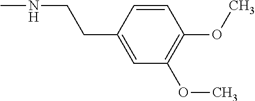 |
| Me | CH₂OEt | 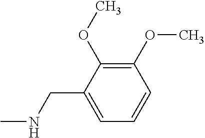 |
| Me | CH₂OEt | 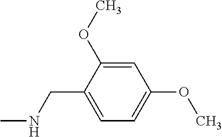 |
| Me | CH₂OEt | 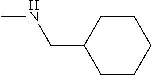 |
| Me | CH₂OEt | 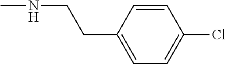 |
| Me | CH₂OEt | 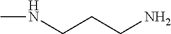 |
| Me | CH₂OEt | 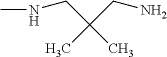 |
| Me | CH₂OEt | 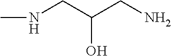 |
| Me | CH₂OEt | 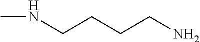 |
| Me | CH₂OEt | 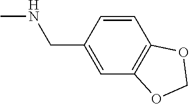 |
| Me | CH₂OEt | 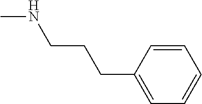 |
| Me | CH₂OEt | 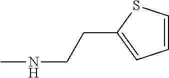 |
| Me | CH₂OEt | 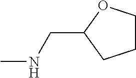 |
| Me | CH₂OEt | 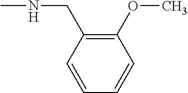 |
| Me | CH₂OEt | 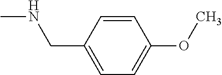 |
| Me | CH₂OEt | 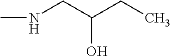 |
| Me | CH₂OEt | 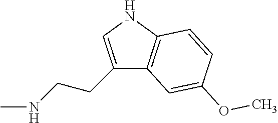 |
| Me | CH₂OEt | 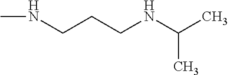 |
| Me | CH₂OEt | 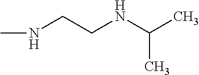 |
| Me | CH₂OEt | 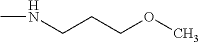 |
| Me | CH₂OEt | 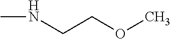 |
| Me | CH₂OEt | 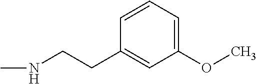 |
| Me | CH₂OEt | 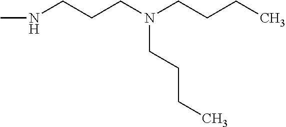 |
| Me | CH₂OEt | 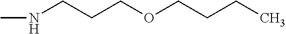 |
| Me | CH₂OEt | 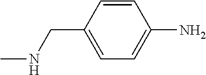 |
| Me | CH₂OEt | 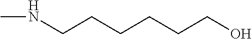 |
| Me | CH₂OEt | 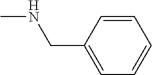 |
| Me | CH₂OEt | 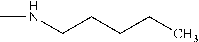 |
| Me | CH₂OEt | 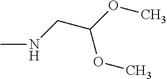 |
| Me | CH₂OEt | 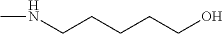 |
| Me | CH₂OEt | 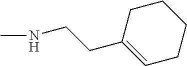 |
| Me | CH₂OEt | 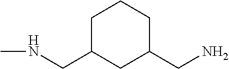 |
| Me | CH₂OEt | 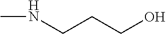 |
| Me | CH₂OEt | 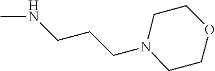 |
| Me | CH₂OEt | 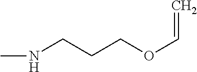 |
| Me | CH₂OEt | 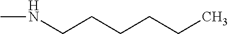 |
| Me | CH₂OEt | 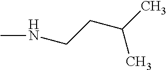 |
| Me | CH₂OEt | 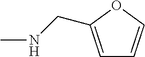 |
| Me | CH₂OEt | 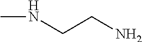 |
| Me | CH₂OEt | 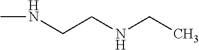 |
| Me | CH₂OEt | 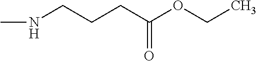 |
| Me | CH₂OEt | 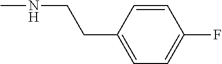 |
| Me | CH₂OEt | 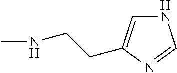 |
| Me | CH₂OEt | 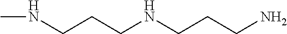 |
| Me | CH₂OEt | 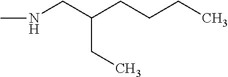 |
| Me | CH₂OEt | 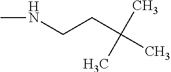 |
| Me | CH₂OEt | 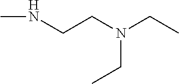 |
| Me | CH₂OEt | 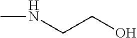 |
| Me | CH₂OEt | 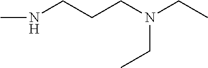 |
| Me | CH₂OEt | 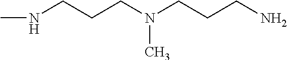 |
| Me | CH₂OEt | 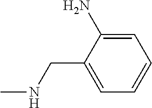 |
| Me | CH₂OEt | 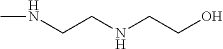 |
| Me | CH₂OEt | 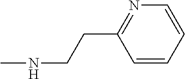 |
| Me | CH₂OEt | 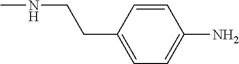 |
| Me | CH₂OEt | 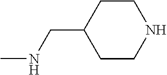 |
| Me | CH₂OEt | 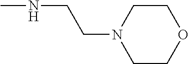 |
| Me | CH₂OEt | 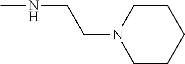 |
| Me | CH₂OEt | 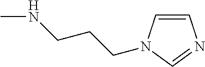 |
| Me | CH₂OEt | 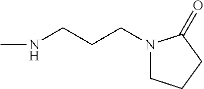 |
| Me | CH₂OEt | 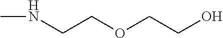 |
| Me | CH₂OEt | 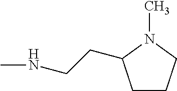 |
| Me | CH₂OEt | 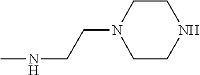 |
| Me | CH₂OEt | 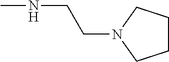 |
| Me | CH₂OEt | 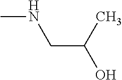 |
| Me | CH₂OEt | 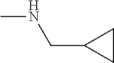 |
| Me | CH₂OEt | 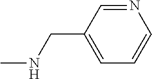 |
| Me | CH₂OEt | 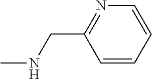 |
| Me | CH₂OEt | 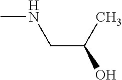 |
| Me | CH₂OEt | 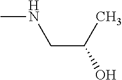 |
| Me | CH₂OEt | 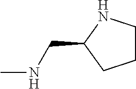 |
| Me | CH₂OEt | 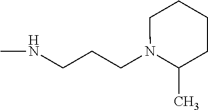 |
| Me | CH₂OEt | 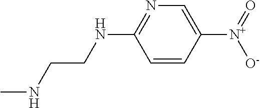 |
| Me | CH₂OEt | 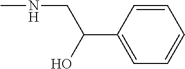 |
| Me | CH₂OEt | 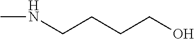 |
| Me | CH₂OEt | 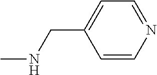 |
| Me | CH₂OEt | 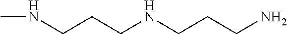 |
| Me | CH₂OEt | 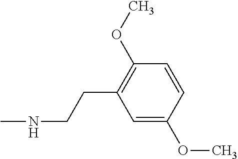 |
| Me | CH₂OEt | 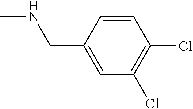 |
| Me | CH₂OEt | 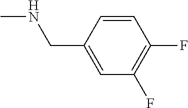 |
| Me | CH₂OEt | 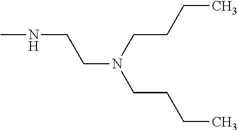 |
| Me | CH₂OEt | 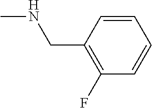 |
| Me | CH₂OEt | 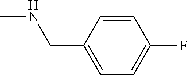 |
| Me | CH₂OEt | 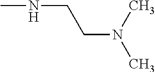 |
| Me | CH₂OEt | 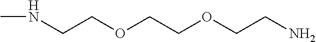 |
| Me | CH₂OEt | 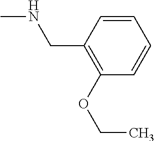 |
| Me | CH₂OEt | 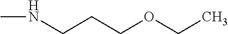 |
| Me | CH₂OEt | 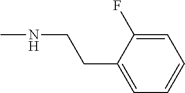 |
| Me | CH₂OEt | 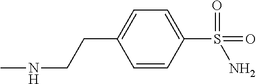 |
| Me | CH₂OEt | 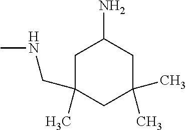 |
| Me | CH₂OEt | 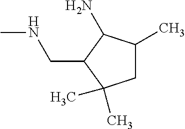 |
| Me | CH₂OEt | 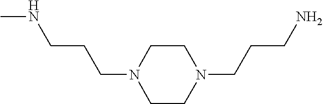 |
| Me | CH₂OEt | 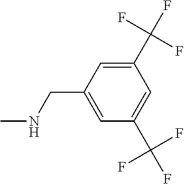 |
| Me | CH₂OEt | 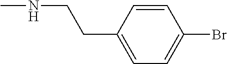 |
| Me | CH₂OEt | 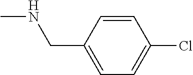 |
| Me | CH₂OEt | 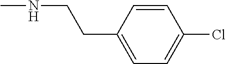 |
| Me | CH₂OEt | 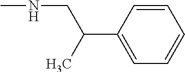 |
| Me | CH₂OEt | 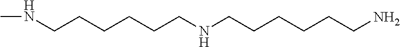 |
| Me | CH₂OEt | 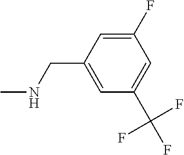 |
| Me | CH₂OEt | 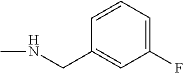 |
| Me | CH₂OEt | 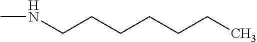 |
| Me | CH₂OEt | 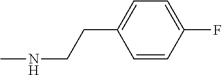 |
| Me | CH₂OEt | 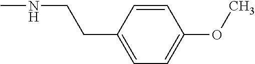 |
| Me | CH₂OEt | 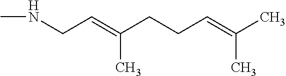 |
| Me | CH₂OEt | 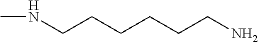 |
| Me | CH₂OEt | 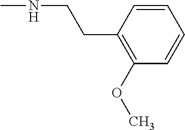 |
| Me | CH₂OEt | 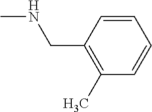 |
| Me | CH₂OEt | 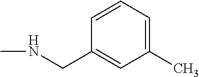 |
| Me | CH₂OEt | 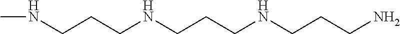 |
| Me | CH₂OEt | 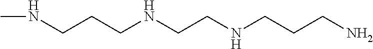 |
| Me | CH₂OEt | 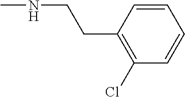 |
| Me | CH₂OEt | 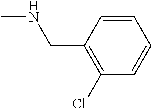 |
| Me | CH₂OEt | 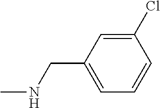 |
| Me | CH₂OEt | 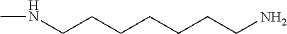 |
| Me | CH₂OEt | 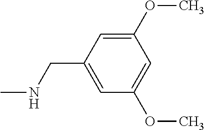 |
| Me | CH₂OEt | 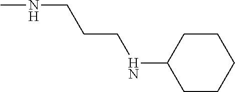 |
| Me | CH₂OEt | 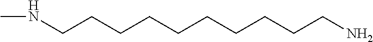 |
| Me | CH₂OEt | 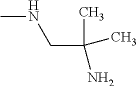 |
| Me | CH₂OEt | 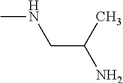 |
| Me | CH₂OEt | 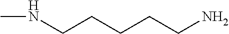 |
| Me | CH₂OEt | 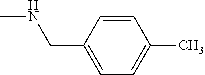 |
| Me | CH₂OEt | 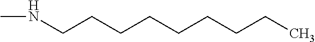 |
| Me | CH₂OEt | 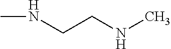 |
| Me | CH₂OEt | 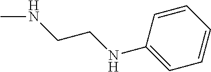 |
| Me | CH₂OEt | 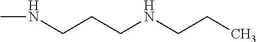 |
| Me | CH₂OEt | 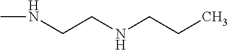 |
| Me | CH₂OEt | 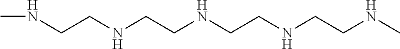 |
| Me | CH₂OEt | 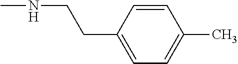 |
| Me | CH₂OEt | 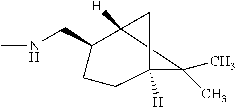 |
| Me | CH₂OEt | 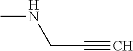 |
| Me | CH₂OEt | 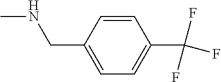 |
| Me | CH₂OEt | 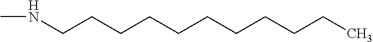 |
| Me | CH₂OEt | 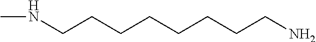 |
| Me | CH₂OEt | 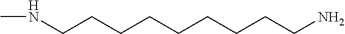 |
| Me | CH₂OEt | 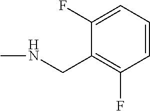 |
| Me | CH₂OEt | 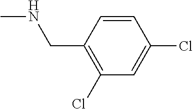 |
| Me | CH₂OEt | 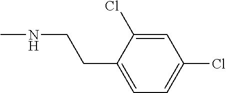 |
| Me | CH₂OEt | 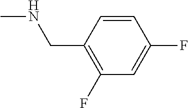 |
| Me | CH₂OEt | 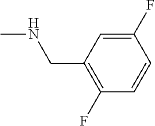 |
| Me | CH₂OEt | 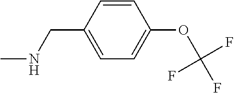 |
| Me | CH₂OEt | 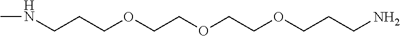 |
| Me | CH₂OEt | 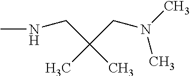 |
| Me | CH₂OEt | 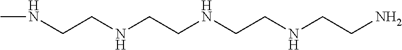 |
| Me | CH₂OEt | 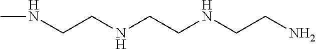 |
| Me | CH₂OEt | 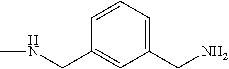 |
| Me | CH₂OEt | 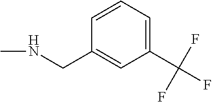 |
| 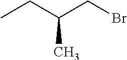 | Me | H |
| 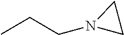 | Me | H |
| 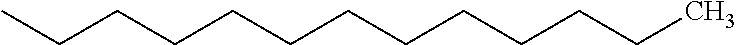 | Me | H |
| 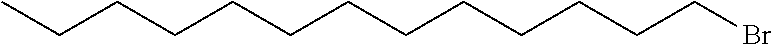 | Me | H |
| 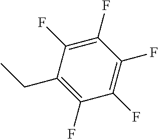 | Me | H |
| 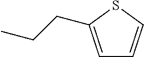 | Me | H |
| 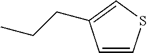 | Me | H |
| 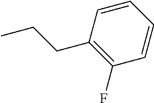 | Me | H |
| 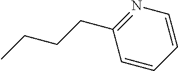 | Me | H |
| 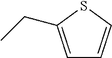 | Me | H |
| 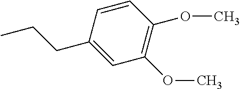 | Me | H |
| 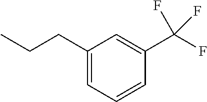 | Me | H |
| 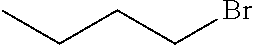 | Me | H |
| 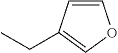 | Me | H |
| 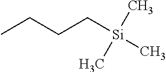 | Me | H |
| 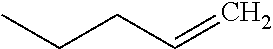 | Me | H |
| 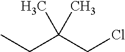 | Me | H |
| 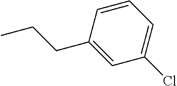 | Me | H |
| 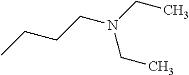 | Me | H |
| 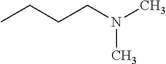 | Me | H |
| 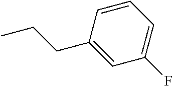 | Me | H |
| 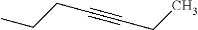 | Me | H |
| 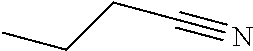 | Me | H |
| 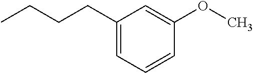 | Me | H |
| 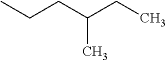 | Me | H |
| 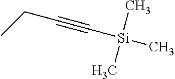 | Me | H |
| 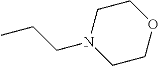 | Me | H |
| 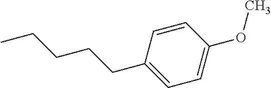 | Me | H |
| 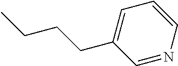 | Me | H |
| 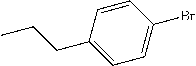 | Me | H |
| 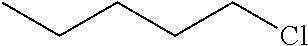 | Me | H |
| 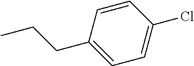 | Me | H |
| 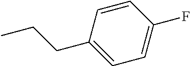 | Me | H |
| 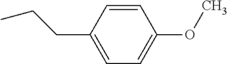 | Me | H |
| 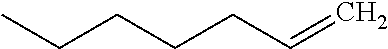 | Me | H |
| 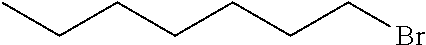 | Me | H |
| 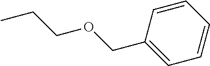 | Me | H |
| 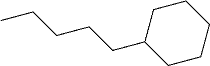 | Me | H |
| 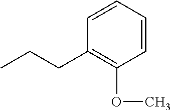 | Me | H |
| 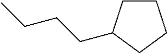 | Me | H |
| 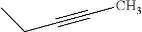 | Me | H |
| 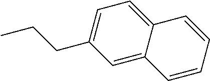 | Me | H |
| 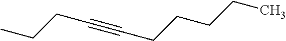 | Me | H |
| 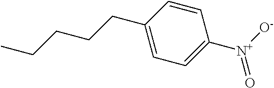 | Me | H |
| 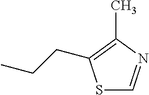 | Me | H |
| 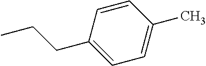 | Me | H |
| 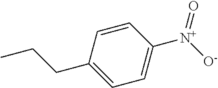 | Me | H |
| 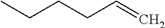 | Me | H |
| 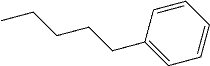 | Me | H |
| 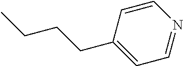 | Me | H |
| 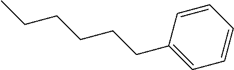 | Me | H |
| 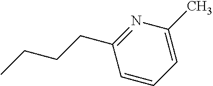 | Me | H |
| 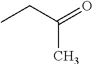 | Me | H |
| 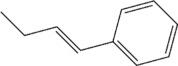 | Me | H |
| 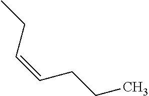 | Me | H |
| 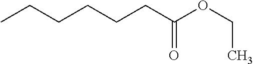 | Me | H |
| 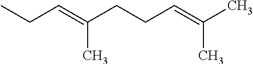 | Me | H |
| 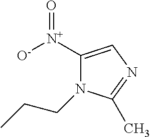 | Me | H |
| 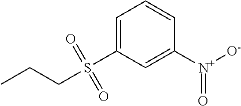 | Me | H |
| 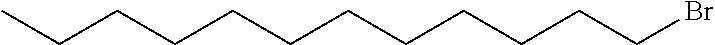 | Me | H |
| 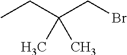 | Me | H |
| 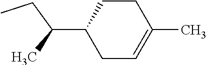 | Me | H |
| 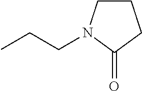 | Me | H |
| 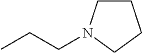 | Me | H |
| 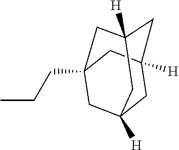 | Me | H |
| 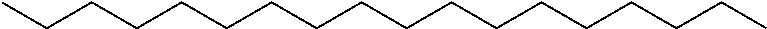 | Me | H |
| 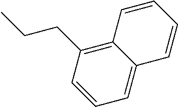 | Me | H |
| 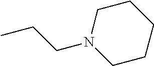 | Me | H |
| 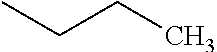 | Me | H |
| 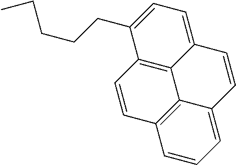 | Me | H |
| 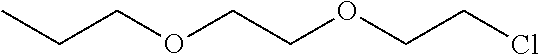 | Me | H |
| 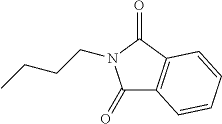 | Me | H |
| 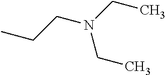 | Me | H |
| 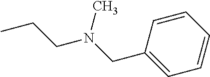 | Me | H |
| 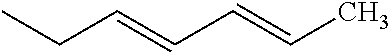 | Me | H |
| 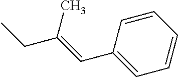 | Me | H |
| 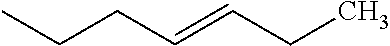 | Me | H |
| 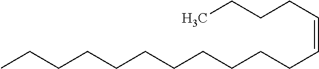 | Me | H |
| 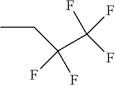 | Me | H |
| 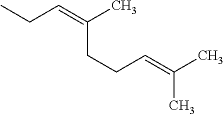 | Me | H |
| 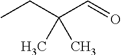 | Me | H |
| 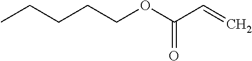 | Me | H |
| 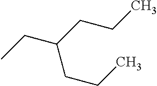 | Me | H |
| 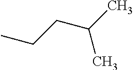 | Me | H |
| 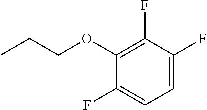 | Me | H |
| 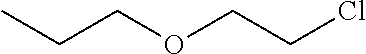 | Me | H |
| 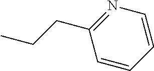 | Me | H |
| 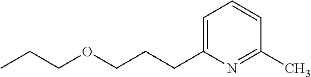 | Me | H |
| 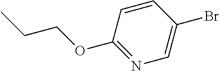 | Me | H |
| 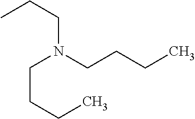 | Me | H |
| 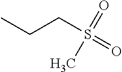 | Me | H |
| 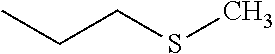 | Me | H |
| 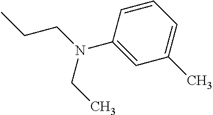 | Me | H |
| 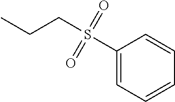 | Me | H |
| 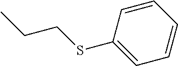 | Me | H |
| 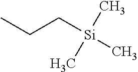 | Me | H |
| 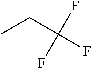 | Me | H |
| 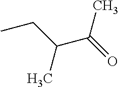 | Me | H |
| 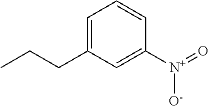 | Me | H |
| 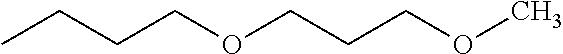 | Me | H |
| 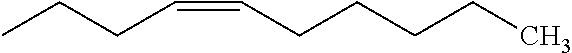 | Me | H |
| 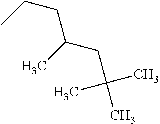 | Me | H |
| 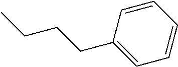 | Me | H |
| 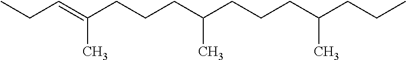 | Me | H |
| 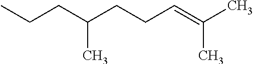 | Me | H |
| 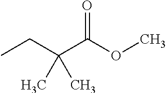 | Me | H |
| 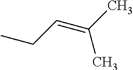 | Me | H |
| 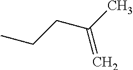 | Me | H |
| 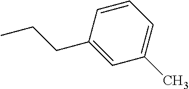 | Me | H |
| 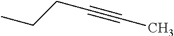 | Me | H |
| 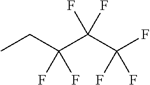 | Me | H |
| 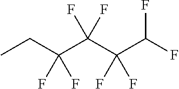 | Me | H |
| 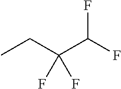 | Me | H |
| 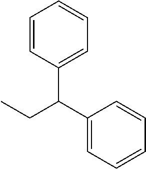 | Me | H |
| 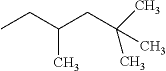 | Me | H |
| 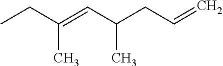 | Me | H |
| 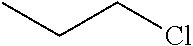 | Me | H |
| 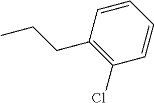 | Me | H |
| 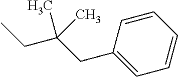 | Me | H |
| 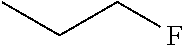 | Me | H |
| 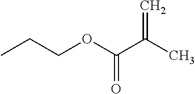 | Me | H |
| 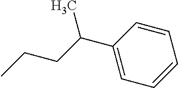 | Me | H |
| 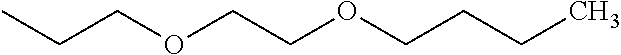 | Me | H |
| 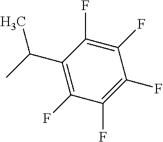 | Me | H |
| 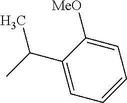 | Me | H |
| 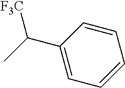 | Me | H |
| 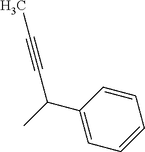 | Me | H |
| 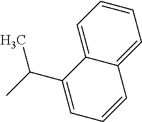 | Me | H |
| 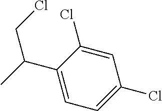 | Me | H |
| 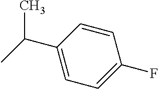 | Me | H |
| 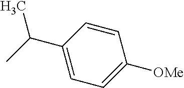 | Me | H |
| 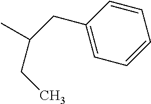 | Me | H |
| 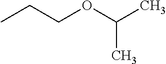 | Me | H |
| 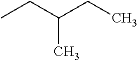 | Me | H |
| 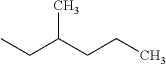 | Me | H |
| 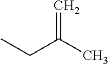 | Me | H |
| 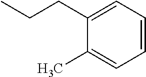 | Me | H |
| 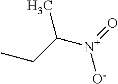 | Me | H |
| 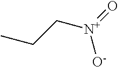 | Me | H |
| 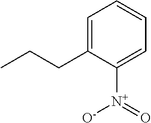 | Me | H |
| 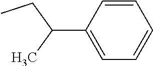 | Me | H |
| 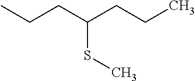 | Me | H |
| 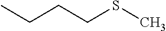 | Me | H |
| 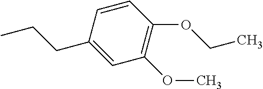 | Me | H |
| 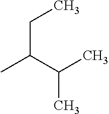 | Me | H |
| 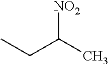 | Me | H |
| 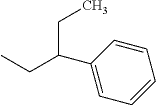 | Me | H |
| 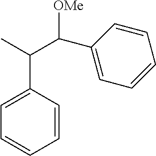 | Me | H |
| 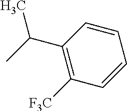 | Me | H |
| 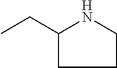 | Me | H |
| 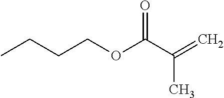 | Me | H |
| 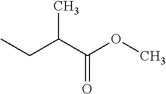 | Me | H |
| 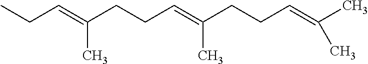 | Me | H |
| 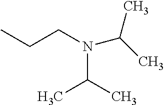 | Me | H |
| 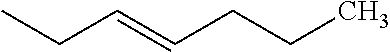 | Me | H |
| Me | 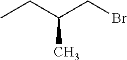 | H |
| Me | 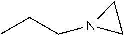 | H |
| Me | 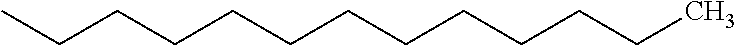 | H |
| Me | 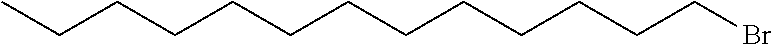 | H |
| Me | 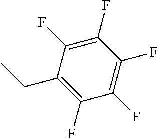 | H |
| Me | 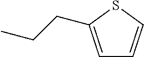 | H |
| Me | 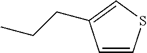 | H |
| Me | 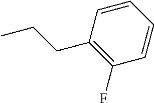 | H |
| Me | 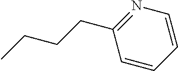 | H |
| Me | 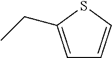 | H |
| Me | 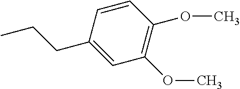 | H |
| Me | 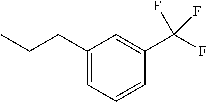 | H |
| Me | 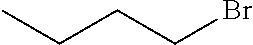 | H |
| Me | 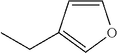 | H |
| Me | 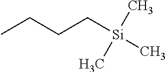 | H |
| Me | 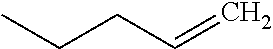 | H |
| Me | 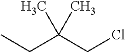 | H |
| Me | 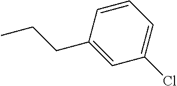 | H |
| Me | 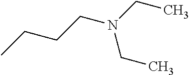 | H |
| Me | 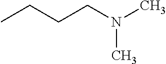 | H |
| Me | 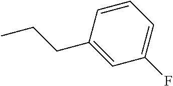 | H |
| Me | 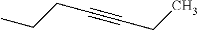 | H |
| Me | 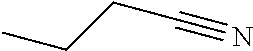 | H |
| Me | 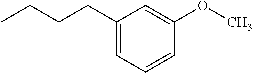 | H |
| Me | 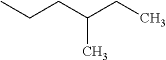 | H |
| Me | 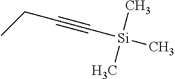 | H |
| Me | 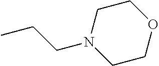 | H |
| Me | 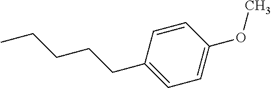 | H |
| Me | 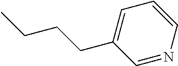 | H |
| Me | 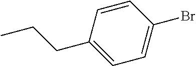 | H |
| Me | 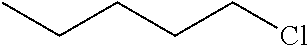 | H |
| Me | 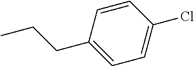 | H |
| Me | 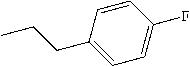 | H |
| Me | 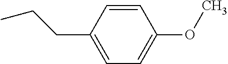 | H |
| Me | 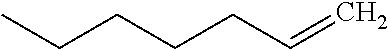 | H |
| Me | 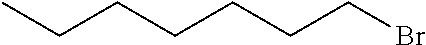 | H |
| Me | 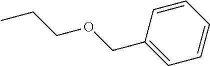 | H |
| Me | 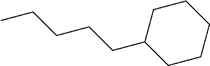 | H |
| Me | 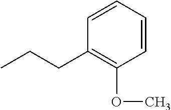 | H |
| Me | 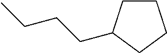 | H |
| Me | 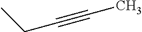 | H |
| Me | 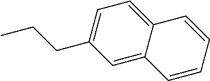 | H |
| Me | 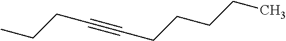 | H |
| Me | 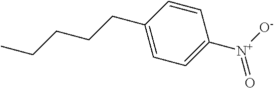 | H |
| Me | 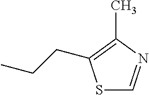 | H |
| Me | 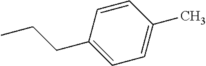 | H |
| Me | 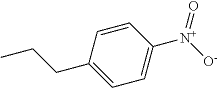 | H |
| Me | 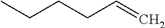 | H |
| Me | 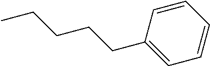 | H |
| Me | 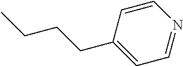 | H |
| Me | 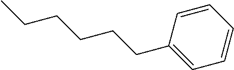 | H |
| Me | 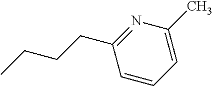 | H |
| Me | 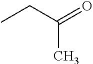 | H |
| Me | 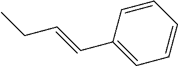 | H |
| Me | 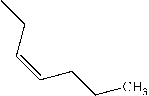 | H |
| Me | 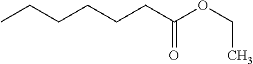 | H |
| Me | 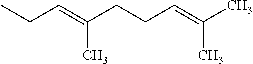 | H |
| Me | 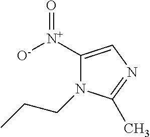 | H |
| Me | 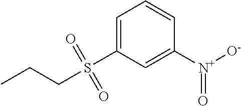 | H |
| Me | 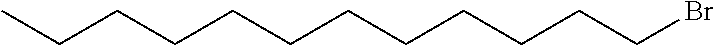 | H |
| Me | 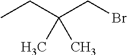 | H |
| Me | 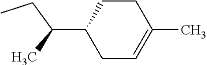 | H |
| Me | 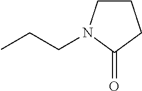 | H |
| Me | 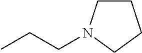 | H |
| Me | 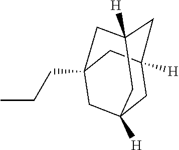 | H |
| Me | 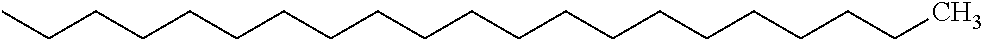 | H |
| Me | 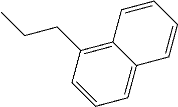 | H |
| Me | 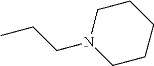 | H |
| Me | 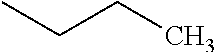 | H |
| Me | 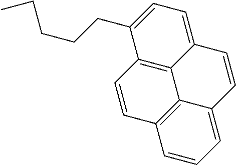 | H |
| Me | 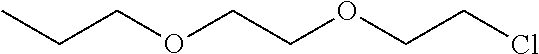 | H |
| Me | 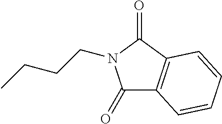 | H |
| Me | 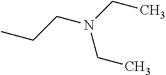 | H |
| Me | 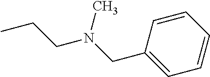 | H |
| Me | 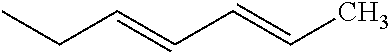 | H |
| Me | 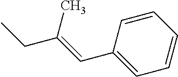 | H |
| Me | 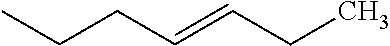 | H |
| Me | 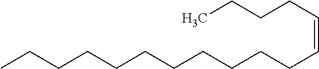 | H |
| Me | 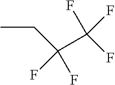 | H |
| Me | 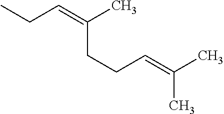 | H |
| Me | 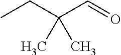 | H |
| Me | 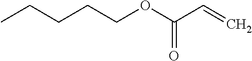 | H |
| Me | 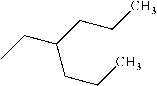 | H |
| Me | 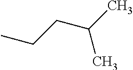 | H |
| Me | 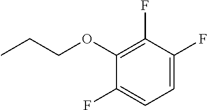 | H |
| Me | 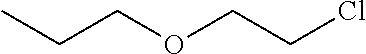 | H |
| Me | 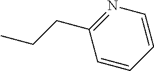 | H |
| Me | 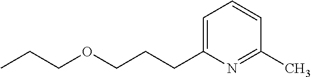 | H |
| Me | 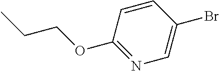 | H |
| Me | 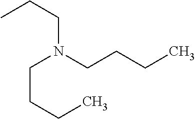 | H |
| Me | 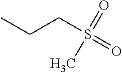 | H |
| Me | 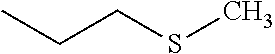 | H |
| Me | 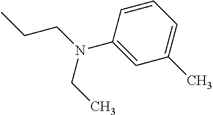 | H |
| Me | 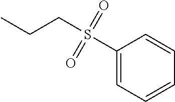 | H |
| Me | 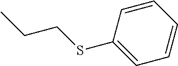 | H |
| Me | 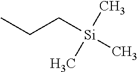 | H |
| Me | 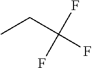 | H |
| Me | 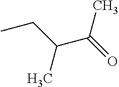 | H |
| Me | 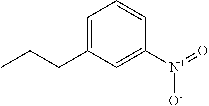 | H |
| Me | 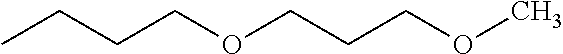 | H |
| Me | 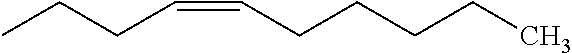 | H |
| Me | 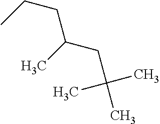 | H |
| Me | 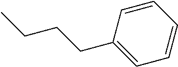 | H |
| Me | 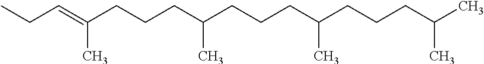 | H |
| Me | 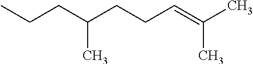 | H |
| Me | 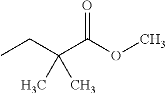 | H |
| Me | 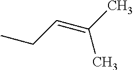 | H |
| Me | 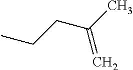 | H |
| Me | 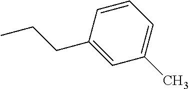 | H |
| Me | 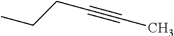 | H |
| Me | 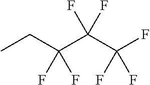 | H |
| Me | 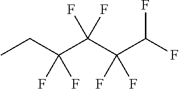 | H |
| Me | 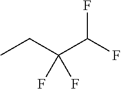 | H |
| Me | 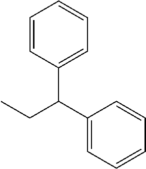 | H |
| Me | 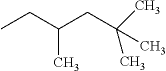 | H |
| Me | 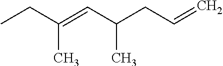 | H |
| Me | 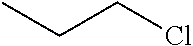 | H |
| Me | 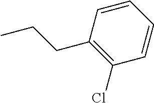 | H |
| Me | 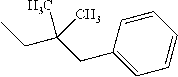 | H |
| Me | 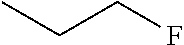 | H |
| Me | 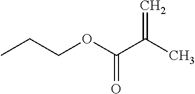 | H |
| Me | 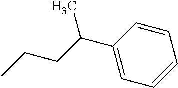 | H |
| Me | 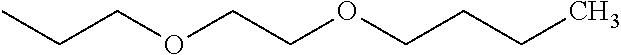 | H |
| Me | 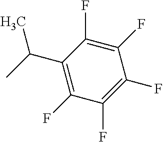 | H |
| Me | 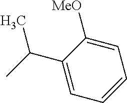 | H |
| Me | 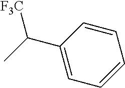 | H |
| Me | 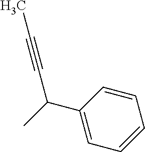 | H |
| Me | 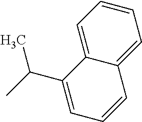 | H |
| Me | 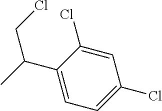 | H |
| Me | 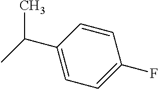 | H |
| Me | 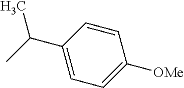 | H |
| Me | 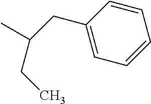 | H |
| Me | 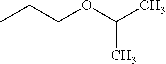 | H |
| Me | 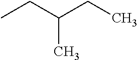 | H |
| Me | 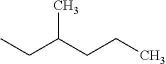 | H |
| Me | 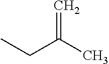 | H |
| Me | 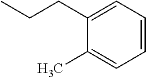 | H |
| Me | 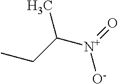 | H |
| Me | 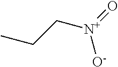 | H |
| Me | 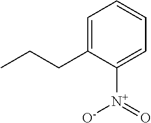 | H |
| Me | 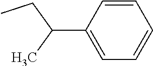 | H |
| Me | 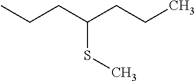 | H |
| Me | 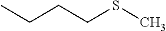 | H |
| Me | 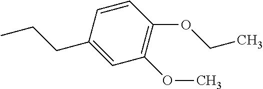 | H |
| Me | 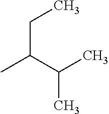 | H |
| Me | 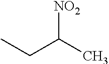 | H |
| Me | 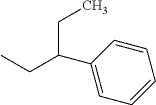 | H |
| Me | 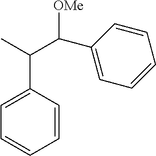 | H |
| Me | 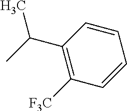 | H |
| Me | 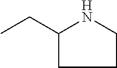 | H |
| Me | 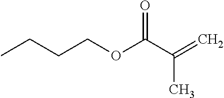 | H |
| Me | 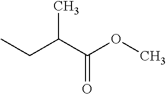 | H |
| Me | 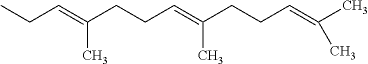 | H |
| Me | 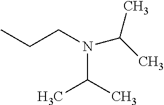 | H |
| Me | 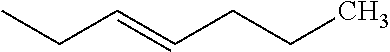 | H |

Accordingly, the invention includes within its scope pharmaceutical compositions comprising, a pharmaceutically acceptable carrier and an active ingredient comprising: (1) a biological/immune response modifier or anti-inflammatory agent (e.g., small molecule, antibody, peptide or gene therapy reagent) that effectively blocks autoimmune response in a mammal by inhibiting the activity or expression of inflammatory cytokines such as, for example, IL-12, IL-23 or IL-27, or members of the Signal Transducers and Activators of Transcription (STAT) family, preferably STAT-4, which are believed to be regulators of T cell differentiation involved in immune responses, alone or in combination with (2) a compound or agent (small molecule or peptide) that facilitates growth and/or differentiation of pancreatic β-cells or any insulin producing cell, either alone or in admixture with a diluent or in the form of a medicament.

In addition to LSF, and the above-described LSF analogs, additional biological/immune response modifying or anti-inflammatory compounds or agents exemplary for use in accordance with the principles of the invention include, without limitation, members of the group consisting of the compounds (LSF analogs) described in the following U.S. patents, the entire disclosures or which are incorporated herein by reference:

| Pat. No. | Title |
|---|---|
| 5,585,380 | Modulation of Cellular Response to External Stimuli |
| 5,648,357 | Enantiomerically Pure Hydroxylated Xanthine Compounds |
| 5,652,243 | Methods of Using Enantiomericallly Pure Hydroxylated |
| | Xanthine Compounds |
| 5,612,349 | Enantiomerically Pure Hydroxylated Xanthine Compounds |
| 5,567,704 | R-Enantiomerically Pure Hydroxylated Xanthine Compounds |
| | To Treat Baldness |
| 5,580,874 | Enantiomerically Pure Hydroxylated Xanthine Compounds |
| 5,739,138 | Enantiomerically Pure Hydroxylated Xanthine Compounds |
| | To Treat Autoimmune Diabetes |
| 5,792,772 | Enantiomerically Pure Hydroxylated Xanthine Compounds |
| 5,620,984 | Enantiomerically Pure Hydroxylated Xanthine Compounds |
| 5,580,873 | Enantiomerically Pure Hydroxylated Xanthine Compounds |
| | To Treat Proliferative Vascular Diseases |
| 5,629,315 | Treatment of Diseases Using Enantiomerically Pure |
| | Hydroxylatsd Xanthine Compounds |
| 5,621,102 | Process for Preparing Enantiomerically Pure Xanthine |
| | Derivatives |
| 5,965,564 | Enantiomerically Pure Hydroxylated Xanthine Compounds |
| 5,629,423 | Asymmetric Synthesis of Chiral Secondary Alcohols |
| 6,780,865 | Compounds Having Selective Hydrolytic Potentials |
| 6,057,328 | Method for Treating Hyperoxia |
| 6,469,017 | Method of Inhibiting Interleukin-12 Signaling |
| 5,288,721 | Substituted Epoxyalkyl Xanthines for Modulation of |
| | Cellular Response |
| 5,866,576 | Expoxide - Containing Compounds |
| 6,121,270 | Epoxide - Containing Compounds |
| 5,340,813 | Substituted Aminoalkyl Xanthines Compounds |
| 5,817,662 | Substituted Amino Alkyl Compounds |
| 5,889,011 | Substituted Amino Alkyl Compounds |
| 6,103,730 | Amine Substituted Compounds |
| 5,801,182 | Amine Substituted Compounds |
| 5,807,861 | Amine Substituted Compounds |
| 5,473,070 | Substituted Long Chain Alcohol Xanthine Compounds |
| 5,804,584 | Hydroxyl-Containing Compounds |
| 5,780,476 | Hydroxyl-Containing Compounds |
| 6,133,274 | Hydroxyl-Containing Bicyclic Compounds |
| 6,693,105 | Hydroxyl-Containing Compounds |
| 6,075,029 | Modulators of Metabolism |
| 5,670,506 | Halogen, Isothiocynate or Azide Substituted Compounds |
| 6,020,337 | Electronegative-Substituted Long Chain Xanthine Compounds |
| 5,795,897 | Oxohexyl Methylxanthine Compounds |
| 5,770,595 | Oxime Substituted Therapeutic Compounds |
| 5,929,081 | Method for Treating Diseases Mediated by Cellular |
| | Proliferation in Response to PDGF, EGF, FGF and VEGF |
| 5,859,018 | Method for Treating Diseases Mediated by Cellular |
| | Proliferation in Response to PDGF, EGF, FGF and VEGF |
| 5,795,898 | Method for Treating Disesses Mediated by Cellular |
| | Proliferation in Response to PDGF, EGF, FGF and VEGF |
| 6,100,271 | Therapeutic Compounds Containing Xanthinyl |
| 5,807,862 | Therapeutic Compounds |
| 6,043,250 | Methods for Using Therapeutic Compounds Containing |
| | Xanthinyl |
| 6,774,130 | Therapeutic Compounds for Inhibiting Interleukin-12 |
| | Signaling and Methods for Using Same |
| 6,878,715 | Therapeutic Compounds for Inhibiting Interleukin-12 |
| | Signaling and Methods for Using Same |
| 6,586,429 | Tricyclic Fused Xanthine Compounds and Their Uses |
| | (As Amended) |

Still further, additional biological/immune response modifying (immunomodulating) or anti-inflammatory compounds or agents that may be for used in accordance with the principles of the invention include, without limitation, members of the group consisting of the following cytokine formation blocking agents or methods: SiRNA (small interfering RNA); mTOR (mammalian target of Rapamycin); Leflunomide and active metabolites (e.g., A77 1726, LEF M); blockers of formation of advance glycation end products or small molecule or antibodies that inhibit the receptor for advance glycation end products (RAGE); Lipoxins or analogs thereof (e.g., LXA4); small molecule inhibitors of IL-12 (e.g., apilimod mesylate (a.k.a. STA-532), Synta Pharmaceuticals); monoclonal antibodies (e.g., anti-interleukin-12 monoclonal antibody (ABT-874, Abbott Laboratories); various methods for inhibiting cytokines described in Vanderbroeck, K, et alt, “Inhibiting Cytokines of the Interleukin-12 Family: Recent Advances and Novel Challenges,” Journal of Pharmacy and Pharmacology, 56:145-160 (2004), and the like.

The dosage of active ingredient in the pharmaceutical compositions of this invention may be varied; however, it is necessary that the amount of the active ingredient be such that a suitable dosage form is obtained. The selected dosage depends upon the desired therapeutic effect, on the route of administration, and on the duration of the treatment. In general, an effective dosage for the activities of this invention is in the range of 1×10⁻⁷ to 200 mg/kg/day, preferably 1×10⁻⁴ to 100 mg/kg/day, which can be administered as a single dose or divided into multiple doses. Preferably the therapeutic amount is between about 0.5 mg to about 12 mg, and more preferably between about 2 mg to about 8 mg, with the most exemplary dosage being between about 2 mg and about 6 mg. Unit dosage forms are preferred.

Generally, a therapeutically effective daily dose is from about 0.001 mg to about 15 mg/kg of body weight per day of a compound of the invention; preferably, from about 0.1 mg to about 10 mg/kg of body weight per day; and most preferably, from about 0.1 mg to about 1.5 mg/kg of body weight per day. For example, for administration to a 70 kg person, the dosage range would be from about 0.07 mg to about 1050 mg per day of a compound of the invention, preferably from about 7.0 mg to about 700 mg per day, and most preferably from about 7.0 mg to about 105 mg per day. Some degree of routine dose optimization may be required to determine an optimal dosing level and pattern. Suitable dosages are well known or readily determinable by the skilled artisan. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

To practice the method of the invention, the pharmaceutical compositions of the invention can be administered by oral, parenteral (e.g., intramuscular, intraperitoneal, intravenous or subcutaneous injection, or implant), by inhalation spray, nasal, buccal, vaginal, rectal, implanted reservoir, sublingual or topical routes of administration and can be formulated with pharmaceutically acceptable carriers to provide dosage forms appropriate for each route of administration. The term “parenteral” as used herein includes subcutaneous, intracutaneous, intravenous, intramuscular, intrarticular, intraarterial, intrasynovial, intrasternal, intrathecal, intralesional and intracranial injection or infusion techniques. Any method of the invention can comprise administering an effective amount of a composition or pharmaceutical composition comprising (1) a biological/immune response modifier or anti-inflammatory agent (e.g., small molecule, antibody, peptide or gene therapy reagent) that effectively blocks autoimmune response or cytokine formation in a mammal (e.g., LSF analogs, as further described herein), alone or in combination with (2) any compound or agent (e.g., small molecule or peptide) (e.g., Ex-4) that facilitates growth and/or differentiation of pancreatic β-cells or any insulin producing cell.

Solid dosage forms for oral administration include capsules, tablets, pills, powders and granules. In such solid dosage forms, the active compound is admixed with at least one inert pharmaceutically acceptable carrier such as sucrose, lactose, or starch. Such dosage forms can also comprise, as is normal practice, additional substances other than such inert diluents, e.g., lubricating agents such as magnesium stearate. In the case of capsules, tablets and pills, the dosage forms may also comprise buffering agents. Tablets and pills can additionally be prepared with enteric coatings.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring and perfuming agents.

Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use.

Compositions for rectal or vaginal administration are preferably suppositories which may contain, in addition to the active substance, excipients such as coca butter or a suppository wax.

Compositions for nasal or sublingual administration are also prepared with standard excipients well known in the art.

The composition of the invention may include also conventional excipients of the type used in pharmaceutical compositions. For example, the composition may include pharmaceutically acceptable organic or inorganic carriers suitable for oral administration. Examples of such carriers include: sugar spheres, diluents, hydrophilic polymers, lubricants, glidants (or anti-adherents), plasticizers, binders, disintegrants, surfactants and pH modifiers.

Suitable diluents include microcrystalline cellulose, lactose, sucrose, fructose, glucose dextrose, or other sugars, dibasic calcium phosphate, calcium sulphate, cellulose, ethylcellulose, cellulose derivatives, kaolin, mannitol, lactitol, maltitol, xylitol, sorbitol, or other sugar alcohols, dry starch, dextrin, maltodextrin or other polysaccharides, inositol or mixtures thereof.

Suitable hydrophilic polymers include hydroxypropylmethyl cellulose, carbomers, polyethylene oxides, hydroxypropyl cellulose, hydroxyethyl cellulose, carboxymethylcellulose, sodium carboxymethylcellulose, carboxyvinylpolymers, polyvinyl alcohols, glucans, scleroglucans, mannans, xanthans, carboxymethylcellulose and its derivatives, methylcellulose and, in general, cellulose, crosslinked polyvinylpyrrolidone, carboxymethyl starch, potassium methacrylate-divinylbenzene copolymer, hydroxypropylcyclodextrin, alpha, beta, gamma cyclodextrin or derivatives and other dextran derivatives, natural gums, seaweed extract, plant exudate, agar, agarose, algin, sodium alginate, potassium alginate, carrageenan, kappa-carrageenan, lambda-carrageenan, fucoidan, furcellaran, laminarin, hypnea, eucheuma, gum arabic, gum ghatti, gum karaya, gum tragacanth, guar gum, locust bean gum, quince psyllium, flax seed, okra gum, arabinogalactin, pectin, scleroglucan, dextran, amylose, amylopectin, dextrin, acacia, karaya, guar, a swellable mixture of agar and carboxymethyl cellulose; a swellable composition comprising methyl cellulose mixed with a sparingly cross-linked agar; a blend of sodium alginate and locust bean gum; and the like.

Suitable glidants (or anti-adherents) include colloidal silica, fumed silicon dioxide, silica hydrogel, talc, fumed silica, gypsum, kaolin and glyceryl monostearate.

Suitable plasticizers include acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitale, di-2-ethylhexyl adipate, di-2-ethylhexyl sebacate, di-2-ethylhexyl azelate, dibutyl sebacate, glyceryl-1 monocaprylate, glyceryl monocaprate.

Suitable binders include starches, hydroxypropylmethyl cellulose, hydroxypropyl cellulose, ethyl cellulose, polyvinyl pyrrolidone, acacia, guar gum, hydroxyethylcellulose, agar, calcium carrageenan, sodium alginate, gelatin, saccharides (including glucose, sucrose, dextrose and lactose), molasses, extract of Irish moss, panwar gum, ghatti gum, mucilage of isapol husk, carboxymethylcellulose, methylcellulose, veegum, larch arbolactan, polyethylene glycols, waxes and mixtures thereof.

Suitable disintegrants include starches, sodium starch glycollate, crospovidone, croscarmellose, microcrystalline cellulose, low substituted hydroxypropyl cellulose, pectins, potassium mnethacrylate-divinylbenzene copolymer, polyvinylalcohol, thylamide, sodium bicarbonate, sodium carbonate, starch derivatives, dextrin, beta cyclodextrin, dextrin derivatives, magnesium oxide, clays, bentonite and mixtures thereof.

Suitable surfactants include nonionic surfactants such as sorbitan sesquioleate, polyoxyethylene sorbitan monooleate, polyoxyethylene monostearate, glycerol monostearate, propylene glycol monolaurate, polyoxyethylene lauryl other, polyoxyethylene cetyl ether or polyoxyethylene hydrogenated castor oil; and ionic surfactants such as sodium dodecyl sulfate or benzalkonium chloride; and the like.

Suitable pH modifiers include organic acids such as citric acid, fumaric acid, tartaric acid, succinic acid, ascorbic acid, acetic acid, malic acid, glutaric acid and adipic acid; salts of these acids; salts of inorganic acids and magnesium hydroxide.

In general, it has proved advantageous to administer intravenously amounts of from 0.01 mg to 10 mg/kg, suitably 0.05 to 5 mg/kg, of body weight per day and to administer orally 0.05 to 20 mg/kg, suitably 0.5 mg to 5 mg/kg of body weight per day, to achieve effective results. Nevertheless, it can at times be necessary to deviate from those dosage rates, and in particular to do so a function of the nature and body weight of the human or animal subject to be treated, the individual reaction of this subject to the treatment, type of formulation in which the active ingredient is administered, the mode in which the administration is carried out and the point in the progress of the disease or interval at which it is to be administered. Thus, it may in some case suffice to use less than the above-mentioned minimum dosage rate, whilst other cases the upper limit mentioned must be exceeded to achieve the desired results. Where larger amounts are administered, it may be advisable to divide these into several individual administrations over the course of the day.

Article of Manufacture

In another aspect, the invention provides an article of manufacture in accordance with the invention comprises a means for holding a pharmaceutical composition(s), as previously described, suitable for administration to a patient in combination with printed labeling instructions providing a discussion of when or how a particular dosage form should be administered to the patient. The pharmaceutical composition will be contained in any suitable means or container capable of holding and dispensing the dosage form and which will not significantly interact with the composition, and will further be in physical relation with the appropriate labeling advising that the dosage form exhibits an ability, or may be used, to restore β-cell mass and function in a mammal in need thereof. The labeling instructions will be consistent with the methods of treatment as described hereinbefore. For example, the labeling may be associated with a container by any means that maintain a physical proximity of the two. Further, by way of nonlimiting example, they may both be contained in a packaging means such as a box or plastic shrink wrap or may be associated with the instructions being bonded to container such as with glue or adhesive that does not obscure the labeling instructions or other bonding or holding means.

The invention will be further illustrated in the following, non limiting prophetic Example, which is illustrative only and does not limit the claimed invention regarding the materials, conditions, process parameters and the like recited herein.

### Prophetic Example

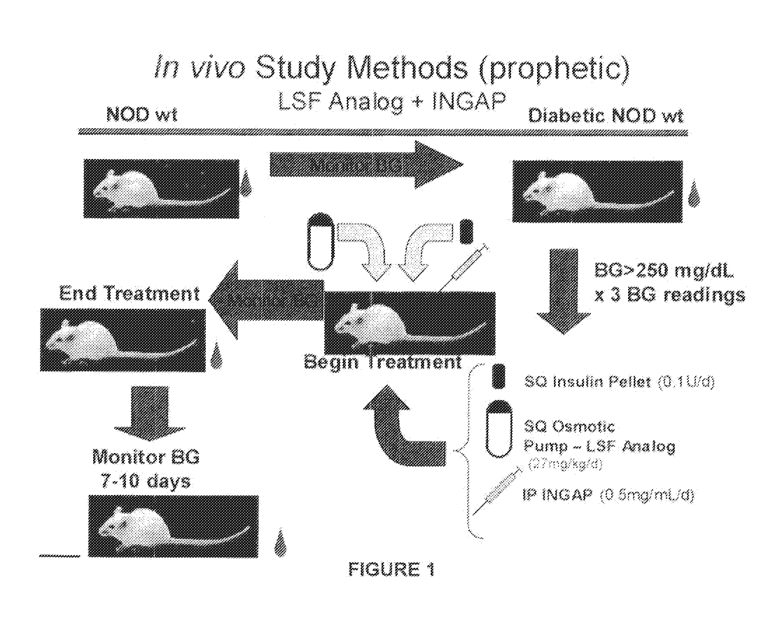

FIG. 1 depicts a prophetic therapy regimen involving INGAP peptide and LSF Analog for building and preserving β-cell mass and increase insulin production in a mammal whereby NOD mice are monitored for diabetes by measuring blood glucose levels. Mice are allowed to develop spontaneous diabetes, which usually occurs around the age of 18 weeks. Non-fasting blood glucose levels >250 mg/dL are measured for 3 different days and are considered evidence of diabetes onset. Shortly after diagnosing diabetes, the NOD mice receive an insulin pellet implanted subcutaneously in order to maintain them during a treatment period of 6 to 7 weeks. The pellet provides 0.1 U/d of bovine insulin. The mice are then assigned to one of 5 treatment groups:

Group 1—Normal saline via continuous subcutaneous infusion (placebo). The saline is delivered via an implantable subcutaneous osmotic mini-pump. The mice receive the placebo for 4 weeks and continue on insulin for another 2 weeks. The insulin is then stopped, and the mice are observed to determine if the treatment was effective.

Group 2—LSF Analog (27 mg/kg/day via continuous subcutaneous infusion) alone. An LSF analog is delivered via an implantable subcutaneous osmotic mini-pump. The mice receive the LSF analog for 4 weeks, continue on insulin for another 2 weeks, and then are observed to determine if the treatment was effective.

Group 3—INGAP (500 μg qd ip) alone. The mice in this group receive INGAP and insulin for 6 weeks, the treatment is then stopped, and the mice is observed to determine if the treatment was effective.

Group 4—LSF Analog (27 mg/kg/day via continuous subcutaneous infusion) plus INGAP (500 μg qd ip) administered concomitantly. The LSF analog is delivered via an implantable subcutaneous osmotic mini-pump. The mice receive LSF for 4 weeks and INGAP and insulin for 6 weeks. The treatments are then stopped and the mice are observed to determine if the treatment was effective.

Group 5—LSF (27 mg/kg/day via continuous subcutaneous infusion) for a week of pretreatment as monotherapy. Here the immune system is first treated to “cool it of” so that any new islets would be growing in a less hostile environment. INGAP is then added to the treatment regimen at (500 μg qd Ip) and administered concomitantly with the LSF for an additional 5 weeks. LSF analog treatment is discontinued at week 6. INGAP and Insulin are continued for 1 more week until week 7. All therapy is then discontinued and the mice are observed to determine if the treatment was effective.

It will be observed that the remission rate with LSF analog pre-treatment is unexpected and superior to protocols without such pre-treatment. The overall remission rate is expected to be almost two times higher or more in the pretreatment group than the concurrent or concomitant group. It is two times higher in those animals with severe diabetes, i.e., starting BG level of over 350. The decrease insulin usage during the treatment period is also expected to be superior. Finally, the quality of the islets being regenerated is expected to be superior in terms of maturation and morphology. There is no remission expected with LSF alone.

In sum, the combination of a biological response modulator (e.g., LSF analog) and a beta cell growth or differentiating factor (e.g., INGAP or Ex-4) following pre-treatment with an immune modulator (e.g., LSF analog) is an effective therapy for T1DM or a disease or condition resulting from the loss of pancreatic islet cells in a patient.

Although the invention has been described in detail with reference to specific embodiments, those of skill in the art will recognize that modifications and improvements are within the scope and spirit of the invention, as set forth in the claims which follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the invention.

What is claimed is:

1. A method for treating diabetes in a mammal, comprising a first step of administering to the mammal a therapeutically-effective amount of a pharmaceutical composition comprising biological response modifier in admixture with a pharmaceutically acceptable carrier, diluent, excipient, adjuvant or vehicle followed by a second step of administering a therapeutically-effective amount of a pharmaceutical composition comprising INGAP in admixture with a pharmaceutically acceptable carrier, diluent, excipient, adjuvant or vehicle.

2. The method of claim 1, wherein the biological response modifier is an immunomodulator including pharmaceutically acceptable enantiomers, diastereomers, tautomers, salts and solvates thereof.

3. The method of claim 1, wherein the biological response modifier comprises a compound, including resolved enantiomers, diastereomers, tautomers, salts and solvates thereof, having the following formula: 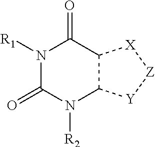
wherein:
X, Y and Z are independently selected from a member of the group consisting of C(R₃), N, N(R₃) and S;
R₁ is selected from a member of the group consisting of hydrogen, methyl, C₍₅₋₉₎alkyl, C₍₅₋₉₎alkenyl, C₍₅₋₉₎alkynyl, C₍₅₋₉₎hydroxyalkyl, C₍₅₋₉₎alkoxyl, C₍₅₋₉₎alkoxyalkyl, the R₁ being optionally substituted;
R₂ and R₃ are independently selected from a member of the group consisting of hydrogen, halo, oxo, C₍₁₋₂₀₎alkyl, C₍₁₋₂₀₎hydroxyalkyl, C₍₁₋₂₀₎thioalkyl, C₍₁₋₂₀₎alkylamino, C₍₁₋₂₀₎alkylaminoalkyl, C₍₁₋₂₀₎aminoalkyl, C₍₁₋₂₀₎aminoalkoxyalkenyl, C₍₁₋₂₀₎aminoalkoxyalkynyl, C₍₁₋₂₀₎diaminoalkyl, C₍₁₋₂₀₎triaminoalkyl, C₍₁₋₂₀₎tetraaminoakyl, C₍₅₋₁₅₎aminotrialkoxyamino, C₍₁₋₂₀₎alkylamido, C₍₁₋₂₀₎alkylamidoalkyl, C₍₁₋₂₀₎amidoalkyl, C₍₁₋₂₀₎acetamidoalkyl, C₍₁₋₂₀₎alkenyl, C₍₁₋₂₀₎alkynyl, C₍₃₋₈₎alkoxyl, C₍₁₋₁₁₎alkoxyalkyl, and C₍₁₋₂₀₎dialkoxyalkyl.

4. The method of claim 3, wherein R₁ is substituted with a member of the group consisting of N—OH, acylamino, cyano group, sulfo, sulfonyl, sulfinyl, sulfhydryl (mercapto), sulfeno, sulfanilyl, sulfamyl, sulfamino, and phosphino, phosphinyl, phospho, phosphono and —NR^{a}R^{b}, wherein each of R^{a} and R^{b} may be the same or different and each is selected from the group consisting of hydrogen, optionally substituted alkyl, cycloalkyl, alkenyl, cycloalkenyl, alkynyl, aryl, heteroaryl and heterocyclic group.

5. The method of claim 3, wherein R₂ and R₃ are selected from the group consisting of methyl, ethyl, oxo, isopropyl, n-propyl, isobutyl, n-butyl, t-butyl, 2-hydroxyethyl, 3-hydroxypropyl, 3-hydroxy-n-butyl, 2-methoxyethyl, 4-methoxy-n-butyl, 5-hydroxyhexyl, 2-bromopropyl, 3-dimethylaminobutyl, 4-chloropentyl, methylamino, aminomethyl, and methylphenyl.

6. The method of claim 3, wherein each R₂ and R₃ is substituted with one or more members of the group consisting of hydroxyl, methyl, carboxyl, furyl, furfuryl, biotinyl, phenyl, naphthyl, amino group, amido group, carbamyl group, cyano group, sulfo, sulfonyl, sulfinyl, sulfhydryl, sulfeno, sulfanilyl, sulfamyl, sulfamino, phosphino, phosphinyl, phospho, phosphono, N—OH, —Si(CH₃)₃, C₍₁₋₃₎alkyl, C₍₁₋₃₎hydroxyalkyl, C₍₁₋₃₎thioalkyl, C₍₁₋₃₎alkylamino, benzyldihydrocinnamoyl group, benzoyldihydrocinnamido group, optionally substituted heterocyclic group and optionally substituted carbocyclic group.

7. The method of claim 3, wherein the heterocyclic group or carbocyclic group is substituted with one or more members of the group consisting of halo, hydroxyl, nitro, SO₂NH₂, C₍₁₋₆₎alkyl, C₍₁₋₆₎haloalkyl, C₍₁₋₈₎alkoxyl, C₍₁₋₁₁₎alkoxyalkyl, C₍₁₋₆₎alkylamino, and C₍₁₋₆₎aminoalkyl.

8. The method of claim 7, wherein the heterocyclic group is a member selected from the group consisting of acridinyl, aziridinyl, azocinyl, azepinyl, benzimidazolyl, benzodioxolanyl, benzofuranyl, benzothiophenyl, carbazole, 4a H-carbazole, chromanyl, chromenyl, cinnolinyl, decahydroquinolinyl, dioxoindolyl, furazanyl, furyl, furfuryl, imidazolidinyl, imidazolinyl, imidazolyl, 1H-indazolyl, indolenyl, indolinyl, indolizinyl, indolyl, 3H-indolyl, isobenzofuranyl, isochromanyl, isoindolinyl, isoindolyl, isoquinolinyl, isothiazolyl, isoxazolyl, morpholinyl, naphthalenyl, naphthyridinyl, norbornanyl, norpinanyl, octahydroisoquinolinyl, oxazolidinyl, oxazolyl, oxiranyl, perimidinyl, phenanthridinyl, phenanthrolinyl, phenarsazinyl, phenazinyl, phenothiazinyl, phenoxathiinyl, phenoxazinyl, phenyl, phthalazinyl, piperazinyl, piperidinyl, 4-piperidonyl, piperidyl, pteridinyl, purinyl, pyranyl, pyrazinyl, pyrazolidinyl, pyrazolinyl, pyrazolyl, pyrenyl, pyridazinyl, pyridinyl, pyridyl, pyridyl, pyrimidinyl, pyrrolidinyl, 2-pyrrolidonyl, pyrrolonyl, pyrrolyl, 2H-pyrrolyl, quinazolinyl, 4H-quinolizinyl, quinolinyl, quinoxalinyl, quinuclidinyl, β-carbolinyl, tetrahydrofuranyl, tetrahydroisoquinolinyl, tetrahydroquinolinyl, tetrazolyl, 6H-1,2,5-thiadiazinyl, 2H-,6H-1,5,2-dithiazinyl, thianthrenyl, thiazolyl, thienyl, thiophenyl, triazinyl, xanthenyl and xanthinyl.

9. The method of claim 8, wherein the carbocyclic group is a member selected from the group consisting of adamantyl, anthracenyl, benzamidyl, benzyl, bicyclo[2.2.1]heptanyl, bicyclo[2.2.1]hexanyl, bicyclo[2.2.2]octanyl, bicyclo[3.2.0]heptanyl, bicyclo[4.3.0]nonanyl, bicyclo[4.4.0]decanyl, biphenyl, biscyclooctyl, cyclobutyl, cyclobutenyl, cycloheptyl, cycloheptenyl, cyclohexanedionyl, cyclohexanedionyl, cyclohexenyl, cyclohexyl, cyclooctanyl, cyclopentadienyl, cyclopentanedionyl, cyclopentenyl, cyclopentyl, cyclopropyl, decalinyl, 1,2-diphenylethanyl, indanyl, 1-indanonyl, indenyl, naphthyl, napthialenyl, phenyl, resorcinolyl, stilbenzyl, tetrahydronaphthyl, tetralinyl, tetralonyl, and tricyclododecanyl.

10. The method of claim 1, wherein the biological response modifier is 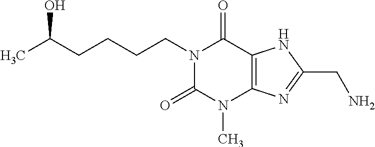 [SMILES: C[C@@H](O)CCCCn1c(=O)c2[nH]c(CN)nc2n(C)c1=O]
or a pharmaceutically acceptable salt thereof.

11. The method of claim 1, wherein the biological response modifier is 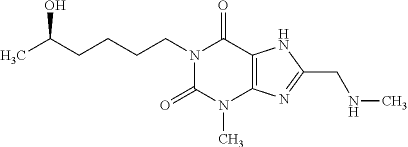 [SMILES: CNCc1nc2c([nH]1)c(=O)n(CCCC[C@@H](C)O)c(=O)n2C]
or a pharmaceutically acceptable salt thereof.

12. The method of claim 1, or pharmaceutically acceptable salt thereof, wherein the biological response modifier is a member selected from the group consisting of: 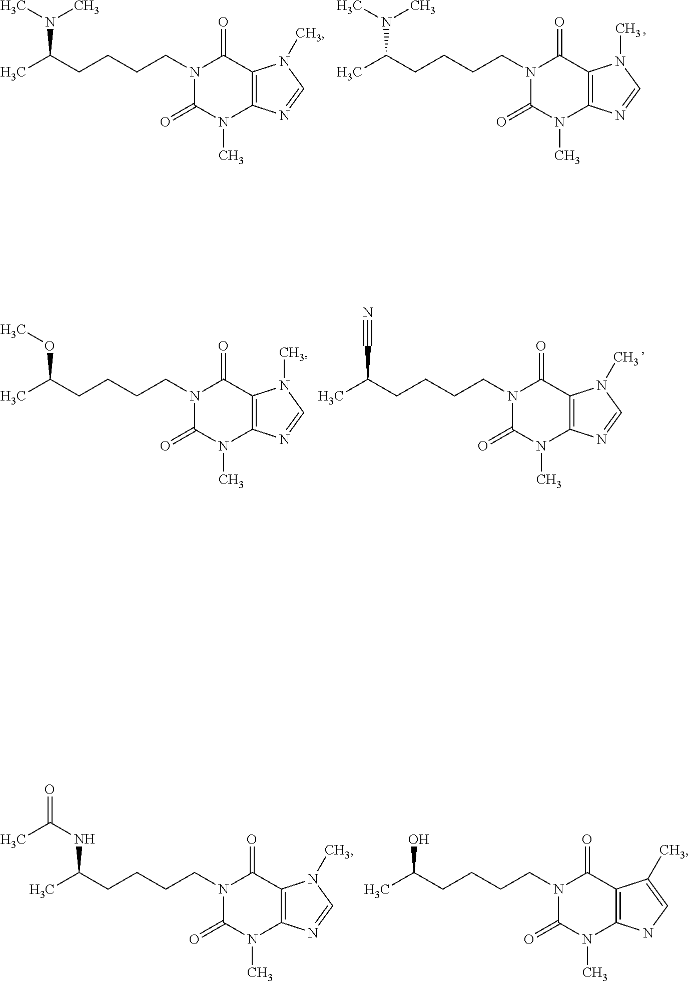 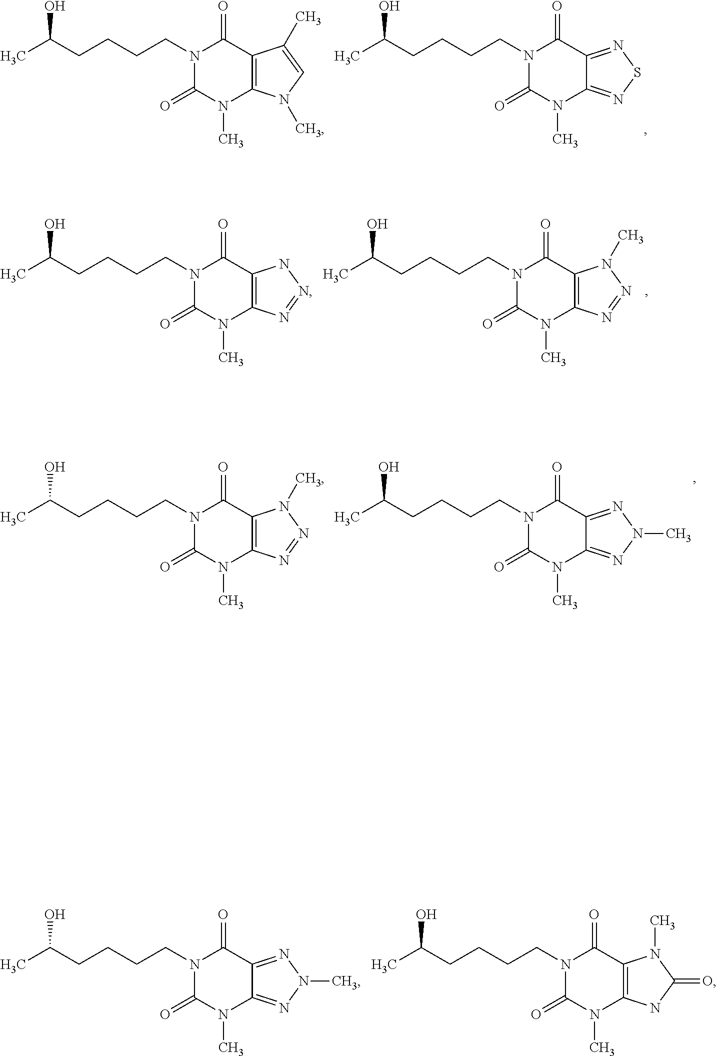 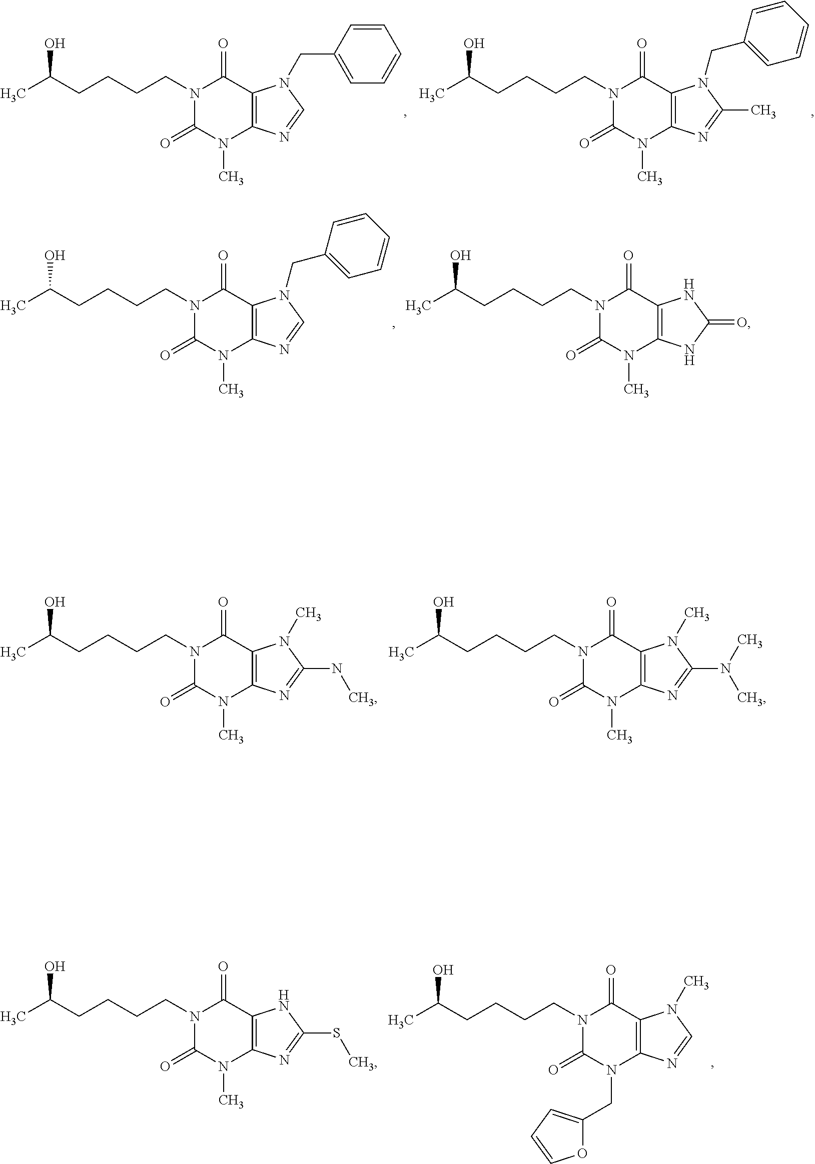 [chemical structure]
